(19) United States
(12) Patent Application Publication
(10) Pub. No.: US 2018/0141939 A1
(43) Pub. Date: May 24, 2018

(54) SOLID FORMS OF A BET INHIBITOR

(71) Applicant: Gilead Sciences, Inc., Foster City, CA (US)

(72) Inventors: Olga Viktorovna Lapina, Newark, CA (US); Ekaterina Albert, West Lafayette, IN (US); Pavel R. Badalov, Edmonton (CA); Jinyu Shen, Edmonton (CA)

(21) Appl. No.: 15/809,347

(22) Filed: Nov. 10, 2017

Related U.S. Application Data
(60) Provisional application No. 62/425,193, filed on Nov. 22, 2016.

(51) Int. Cl. C07D 413/14; A61P 35/00

(52) U.S. Cl. CPC C07D 413/14; C07B 2200/13; A61P 35/00

(57) ABSTRACT

Forms of (2-cyclopropyl-6-(3,5-dimethylisoxazol-4-yl)-1H-benzo[d]imidazol-4-yl)di(pyridin-2-yl)methanol (Compound I) were prepared and characterized in the solid state:

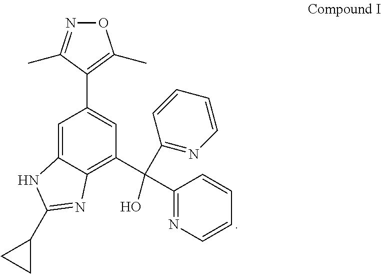 [SMILES: Cc1noc(C)c1-c1cc(C(O)(c2ccccn2)c2ccccn2)c2nc(C3CC3)[nH]c2c1]

Also provided are processes of manufacture and methods of using the forms of Compound I.

### CROSS-REFERENCE TO RELATED APPLICATION

This application claims the benefit of U.S. Application No. 62/425,193, filed on Nov. 22, 2016, which is hereby incorporated by reference in its entirety.

### FIELD

The present disclosure relates generally to solid forms of compounds that modulate or inhibit the activity of bromodomain-containing proteins, pharmaceutical compositions thereof, therapeutic uses thereof, and processes for making the forms.

### BACKGROUND

Therapeutic agents that function as modulators or inhibitors of the bromodomain and extraterminal (BET) family of proteins (e.g., including BRD2, BRD3, BRD4, and BRDT) have the potential to remedy or improve the lives of patients in need of treatment for diseases or conditions such as neurodegenerative, cardiovascular, inflammatory, autoimmune, renal, viral and metabolic disorders. In particular, BET modulators or inhibitors have the potential to treat cancer (including carcinoma, lymphoma, multiple myeloma, leukemia, neoplasms or tumors), rheumatoid arthritis, osteoarthritis, atherosclerosis, psoriasis, systemic lupus erythematosus, multiple sclerosis, inflammatory bowel disease, asthma, chronic obstructive airways disease, pneumonitis, dermatitis, alopecia, nephritis, vasculitis, Alzheimer's disease, hepatitis, primary biliary cirrhosis, sclerosing cholangitis, and diabetes (including type I diabetes) among others. Suitable compounds, including benzimidazole derivatives, for the treatment of such diseases and conditions are disclosed in U.S. Publication No. 2014/0336190, the disclosure of which is incorporated herein by reference in its entirety.

There remains a need for high purity solid forms of benzimidazole derivatives that are efficacious and exhibit improved stability, solubility, and pharmacokinetic or pharmacodynamic profiles for the treatment of diseases modulated by BET proteins.

### SUMMARY

Compound I is known to modulate or inhibit BET activity and is described, for example, in U.S. Publication No. 2014/0336190A1, which is hereby incorporated by reference in its entirety. Compound I has the formula:

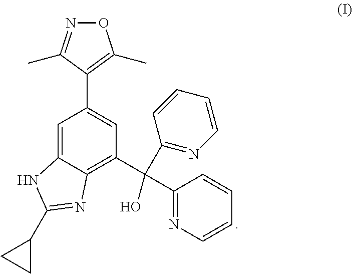 [SMILES: Cc1noc(C)c1-c1cc(C(O)(c2ccccn2)c2ccccn2)c2nc(C3CC3)[nH]c2c1]

The present disclosure provides solid forms of Compound I and complexes (including salts or co-crystals), hydrates, and solvates thereof. Also described herein are processes for making the forms of Compound I, pharmaceutical compositions comprising crystalline forms of Compound I, and methods for using such forms and pharmaceutical compositions in the treatment of diseases mediated by BET proteins.

Accordingly, one embodiment is directed to a phosphate complex of Compound I having a crystalline form.

One embodiment is directed to a phosphate complex of Compound I in a crystalline form characterized by an X-ray powder diffractogram comprising peaks at 5.0, 15.8, and 21.7° 2θ±0.2° 2θ, as determined on a diffractometer using Cu-Kα radiation (Compound I phosphate Form I).

One embodiment is directed to a phosphate complex of Compound I having a crystalline form characterized by an X-ray powder diffractogram comprising peaks at 13.4, 15.0, and 20.2° 2θ±0.2° 2θ, as determined on a diffractometer using Cu-Kα radiation (Compound I phosphate Form II).

One embodiment is directed to a phosphate complex of Compound I having a crystalline form characterized by an X-ray powder diffractogram comprising peaks at 14.8, 19.7, and 24.5° 2θ±0.2° 2θ, as determined on a diffractometer using Cu-Kα radiation (Compound I phosphate Form III).

One embodiment is directed to a phosphate complex of Compound I having a crystalline form characterized by an X-ray powder diffractogram comprising peaks at 9.8, 26.5, and 29.6° 2θ±0.2° 2θ, as determined on a diffractometer using Cu-Kα radiation (Compound I phosphate Form IV).

One embodiment is directed to a phosphate complex of Compound I having a crystalline form characterized by an X-ray powder diffractogram comprising peaks at 12.9, 14.0, and 22.0° 2θ±0.2° 2θ, as determined on a diffractometer using Cu-Kα radiation (Compound I phosphate Form V).

One embodiment is directed to a pharmaceutical composition comprising a form or forms of Compound I or a complex, hydrate, or solvate thereof as described herein, and one or more pharmaceutically acceptable carriers. In one embodiment, the pharmaceutical composition comprises one or more compounds selected from the group consisting of: a phosphate complex of Compound I; Compound I phosphate Form I; Compound I phosphate Form II; Compound I phosphate Form III; Compound I phosphate Form IV; Compound I phosphate Form V, and Compound I phosphate (amorphous) as described herein.

One embodiment is directed to a pharmaceutical composition comprising Compound I phosphate Form I, and one or more pharmaceutically acceptable carriers.

One embodiment is directed to a method of treating a disease mediated, at least in part, by a bromodomain in a patient in need thereof comprising administering a therapeutically effective amount of a form or forms of Compound I or a complex, hydrate, or solvate thereof as described herein. In one embodiment, the bromodomain is a member of the bromodomain and extraterminal (BET) family. In one embodiment, the disease is a cancer of the colon, rectum, prostate, lung, pancreas, liver, kidney, cervix, uterus, stomach, ovary, breast, skin, or the nervous system. In one embodiment, the disease is a cancer of the colon. In one embodiment, the disease is a cancer of the prostate. In one embodiment, the disease is a cancer of the breast. In one embodiment, the disease is a lymphoma. In one embodiment, the disease is a B-cell lymphoma. In one embodiment, the disease is diffuse large B-cell lymphoma.

In one embodiment, the method of treating a disease mediated, at least in part, by a bromodomain in a patient in need thereof comprises administering a therapeutically effective amount of a phosphate complex of Compound I; Compound I phosphate Form I; Compound I phosphate Form II; Compound I phosphate Form III; Compound I phosphate Form IV; Compound I phosphate Form V; Compound I phosphate (amorphous); or a pharmaceutical composition as described herein. In one embodiment, the method of treating a disease mediated, at least in part, by a bromodomain in a patient in need thereof comprises administering a therapeutically effective amount of Compound I phosphate Form I.

### BRIEF DESCRIPTION OF THE DRAWINGS

FIG. 1 shows an X-ray powder diffractogram of Compound I Form I.

FIG. 2 shows a differential scanning calorimeter (DSC) curve of Compound I Form I.

FIG. 3 shows a thermogravimetric analysis (TGA) of Compound I Form I.

FIG. 4 shows an X-ray powder diffractogram of Compound I Form II.

FIG. 5 shows a differential scanning calorimeter (DSC) curve of Compound I Form II.

FIG. 6 shows a thermogravimetric analysis (TGA) of Compound I Form II.

FIG. 7 shows an X-ray powder diffractogram of Compound I Material A, present as a mixture with Compound I Form I.

FIG. 8 shows a differential scanning calorimeter (DSC) curve of Compound I Material A, present as a mixture with Compound I Form I.

FIG. 9 shows a thermogravimetric analysis (TGA) of Compound I Material A, present as a mixture with Compound I Form I.

FIG. 10 shows an X-ray powder diffractogram of Compound I amorphous.

FIG. 11 shows an X-ray powder diffractogram of Compound I phosphate Form I.

FIG. 12 shows a differential scanning calorimeter (DSC) curve of Compound I phosphate Form I.

FIG. 13 shows a thermogravimetric analysis (TGA) of Compound I phosphate Form I.

FIG. 14 shows an X-ray powder diffractogram of Compound I phosphate Form II.

FIG. 15 shows a differential scanning calorimeter (DSC) curve of Compound I phosphate Form II.

FIG. 16 shows a thermogravimetric analysis (TGA) of Compound I phosphate Form II.

FIG. 17 shows an X-ray powder diffractogram of Compound I phosphate Form III.

FIG. 18 shows a differential scanning calorimeter (DSC) curve of Compound I phosphate Form III.

FIG. 19 shows a thermogravimetric analysis (TGA) of Compound I phosphate Form III.

FIG. 20 shows an X-ray powder diffractogram of Compound I phosphate Form IV.

FIG. 21 shows a differential scanning calorimeter (DSC) curve of Compound I phosphate Form IV.

FIG. 22 shows a thermogravimetric analysis (TGA) of Compound I phosphate Form IV.

FIG. 23 shows an X-ray powder diffractogram of Compound I phosphate Form V.

FIG. 24 shows a differential scanning calorimeter (DSC) curve of Compound I phosphate Form V.

FIG. 25 shows a thermogravimetric analysis (TGA) of Compound I phosphate Form V.

FIG. 26 shows an X-ray powder diffractogram of Compound I phosphate amorphous.

FIG. 27 shows X-ray powder diffractograms of Compound I HCl Material A, present as a mixture with Compound I HCl Material B; Compound I HCl Material B; Compound I HCl Material C, present as a mixture with Compound I HCl Material B; Compound I HCl Material D; and Compound I HCl Material E, present as a mixture with Compound I HCl Material D.

FIG. 28 shows an X-ray powder diffractogram of Compound I HCl Material B.

FIG. 29 shows a differential scanning calorimeter (DSC) curve of Compound I HCl Material B.

FIG. 30 shows a thermogravimetric analysis (TGA) of Compound I HCl Material B.

FIG. 31 shows an X-ray powder diffractogram of Compound I HCl Material D.

FIG. 32 shows a differential scanning calorimeter (DSC) curve of Compound I HCl Material D.

FIG. 33 shows a thermogravimetric analysis (TGA) of Compound I HCl Material D.

FIG. 34 shows an X-ray powder diffractogram of Compound I sulfate Material A.

FIG. 35 shows a differential scanning calorimeter (DSC) curve of Compound I sulfate Material A.

FIG. 36 shows a thermogravimetric analysis (TGA) of Compound I sulfate Material A.

FIG. 37 shows an X-ray powder diffractogram of Compound I sulfate Material B.

FIG. 38 shows a differential scanning calorimeter (DSC) curve of Compound I sulfate Material B.

FIG. 39 shows a thermogravimetric analysis (TGA) of Compound I sulfate Material B.

FIG. 40 shows X-ray powder diffractograms of Compound I sulfate Material C, present as a mixture with Compound I sulfate Material A; Compound I sulfate Material A; and Compound I sulfate Material B.

FIG. 41 shows a differential scanning calorimeter (DSC) curve of Compound I sulfate Material C, present as a mixture with Compound I sulfate Material A.

FIG. 42 shows a thermogravimetric analysis (TGA) of Compound I sulfate Material C, present as a mixture with Compound I sulfate Material A.

FIG. 43 shows an X-ray powder diffractogram of Compound I tosylate Form I.

FIG. 44 shows a differential scanning calorimeter (DSC) curve of Compound I tosylate Form I.

FIG. 45 shows a thermogravimetric analysis (TGA) of Compound I tosylate Form I.

FIG. 46 shows an X-ray powder diffractogram of Compound I tosylate Material A.

FIG. 47 shows an X-ray powder diffractogram of Compound I tosylate Material C, present as a mixture with Compound I tosylate Form I.

FIG. 48 shows an X-ray powder diffractogram of Compound I edisylate Material A.

FIG. 49 shows a differential scanning calorimeter (DSC) curve of Compound I edisylate Material A.

FIG. 50 shows a thermogravimetric analysis (TGA) of Compound I edisylate Material A.

FIG. 51 shows an X-ray powder diffractogram of Compound I besylate Material A.

FIG. 52 shows a differential scanning calorimeter (DSC) curve of Compound I besylate Material A.

FIG. 53 shows a thermogravimetric analysis (TGA) of Compound I besylate Material A.

FIG. 54 shows an X-ray powder diffractogram of Compound I mesylate Material A.

FIG. 55 shows an X-ray powder diffractogram of Compound I mesylate Material B.

FIG. 56 shows a differential scanning calorimeter (DSC) curve of Compound I mesylate Material B.

FIG. 57 shows a thermogravimetric analysis (TGA) of Compound I mesylate Material B.

FIG. 58 shows an X-ray powder diffractogram of Compound I mesylate Material C.

FIG. 59 shows a differential scanning calorimeter (DSC) curve of Compound I mesylate Material C.

FIG. 60 shows a thermogravimetric analysis (TGA) of Compound I mesylate Material C.

FIG. 61 shows an X-ray powder diffractogram of Compound I mesylate Material D, present as a mixture with Compound I mesylate Material B.

FIG. 62 shows an X-ray powder diffractogram of Compound I mesylate Material E.

FIG. 63 shows an X-ray powder diffractogram of Compound I mesylate Material F.

FIG. 64 shows an X-ray powder diffractogram of Compound I mesylate Material G.

FIG. 65 shows a differential scanning calorimeter (DSC) curve of Compound I mesylate Material G.

FIG. 66 shows a thermogravimetric analysis (TGA) of Compound I mesylate Material G.

FIG. 67 shows an X-ray powder diffractogram of Compound I napsylate Material A.

FIG. 68 shows an X-ray powder diffractogram of Compound I tartrate Material A.

FIG. 69 shows an X-ray powder diffractogram of Compound I tartrate Material B.

FIG. 70 shows a differential scanning calorimeter (DSC) curve of Compound I tartrate Material B.

FIG. 71 shows a thermogravimetric analysis (TGA) of Compound I tartrate Material B.

FIG. 72 shows an X-ray powder diffractogram of Compound I xinafoate Form I.

FIG. 73 shows a differential scanning calorimeter (DSC) curve of Compound I xinafoate Form I.

FIG. 74 shows a thermogravimetric analysis (TGA) of Compound I xinafoate Form I.

FIG. 75 shows an X-ray powder diffractogram of Compound I gentisate Material A.

FIG. 76 shows a differential scanning calorimeter (DSC) curve of Compound I gentisate Material A.

FIG. 77 shows a thermogravimetric analysis (TGA) of Compound I gentisate Material A.

FIG. 78 shows an X-ray powder diffractogram of Compound I oxalate (disordered).

FIG. 79 shows a solubility profile of Compound I phosphate Form I in DMF/MeCN as a function of temperature.

FIG. 80 shows a solubility profile of Compound I phosphate Form I in DMF/MeCN as a function of the volume percent of DMF.

FIG. 81 shows a solubility profile of Compound I phosphate Form I in DMSO/MeCN as a function of temperature.

FIG. 82 shows polarized light microscopy (PLM) images for seed crystals of Compound I phosphate Form I (FIG. 82a); and the resulting Compound I phosphate Form I crystals formed via Methods 1-3 (FIG. 82(d)-(b), respectively) described herein. The full length of the scale bar in the PLM images is 100 μm.

FIG. 83 shows polarized light microscopy (PLM) images of Compound I phosphate Form I crystals resulting from recrystallization in different ratios of DMF/MeCN (v/v): (a) 50:50; (b) 55:45; (c) 60:40; and (d) 67:33.

FIG. 84 shows a polarized light microscopy (PLM) image of Compound I phosphate Form I having a D₉₀ particle size of about 50 μm.

FIG. 85 shows a polarized light microscopy (PLM) image of Compound I phosphate Form I having a D₉₀ particle size in a range from about 100 μm to about 150 μm.

FIG. 86 shows a polarized light microscopy (PLM) image of Compound I phosphate Form I having a D₉₀ particle size in a range from about 150 μm to about 200 μm.

FIG. 87 shows a pH solubility profile of Compound I.

FIG. 88 shows the chemical stability of Compound I as a function of temperature and pH.

FIG. 89 shows the chemical stability of Compound I in a solution containing 1% hydrogen peroxide (FIG. 89(a)) and iron (II) ions (FIG. 89(b)) as a function of pH.

FIG. 90 shows structures of Compound I products formed under oxidative conditions as indicated via liquid chromatography-mass spectrometry (LC/MC).

FIG. 91 shows X-ray powder diffractograms of Compound I phosphate Form I indicating the chemical stability thereof at various conditions for 1 month.

FIG. 92 shows dissolution profiles (50 mM sodium acetate solution, pH of 5) of Compound I Form I and Compound I phosphate Form I.

FIG. 93 shows pharmacokinetic profile for dogs given a fixed dose of Compound I Form I

FIG. 94 shows pharmacokinetic profile for dogs given a fixed dose of Compound I phosphate Form I.

FIG. 95 shows pharmacokinetic profile for dogs given a tablet comprising a 10 mg dose of Compound I phosphate Form I.

FIG. 96 shows pharmacokinetic profiles for dogs given a tablet comprising Compound I phosphate Form I or a capsule comprising Compound I phosphate Form III.

### DETAILED DESCRIPTION

The compound, (2-cyclopropyl-6-(3,5-dimethylisoxazol-4-yl)-1H-benzo[d]imidazol-4-yl)di(pyridin-2-yl)methanol, designated herein as Compound I or Compound I (free base), has the following formula:

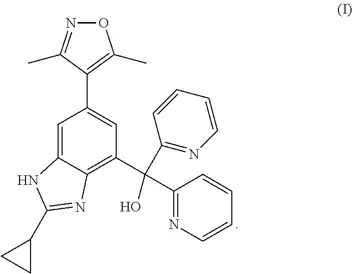 [SMILES: Cc1noc(C)c1-c1cc(C(O)(c2ccccn2)c2ccccn2)c2nc(C3CC3)[nH]c2c1]

Compound I is a selective and potent inhibitor or modulator of BET proteins. The synthesis and method of use thereof is described in U.S. Publication No. 2014/0336190 A1, which is herein incorporated by reference in its entirety.

The present disclosure relates to various solid forms of Compound I, and processes for making such solid forms. For instance, Compound I provides forms further described herein as “Compound I Form I,” “Compound I Form II,” and “Compound I Material A.”

Additional solid forms of Compound I are also described herein, as well as the processes of making such forms. In some embodiments, solid forms of Compound I may include complexes (including salts or co-crystals) of Compound I. Complexes of Compound I may have the following formula:

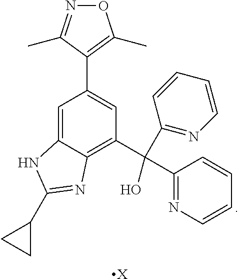 [SMILES: Cc1noc(C)c1-c1cc(C(O)(c2ccccn2)c2ccccn2)c2nc(C3CC3)[nH]c2c1 ; "·X"]

In some embodiments, X may be besylate, edisylate, gentisate, hydrochloride, mesylate, napsylate, oxalate, phosphate, sulfate, tartrate, tosylate, and xinafoate. The following exemplary forms are further described herein: “Compound I besylate Material A,” “Compound I edisylate Form I,” “Compound I gentisate Material A,” “Compound I HCl Material A,” “Compound I HCl Material B,” “Compound I HCl Material C,” “Compound I HCl Material D,” “Compound I HCl Material E,” “Compound I mesylate Material A,” “Compound I mesylate Material B,” “Compound I mesylate Material C,” “Compound I mesylate Material D,” “Compound I mesylate Material E,” “Compound I mesylate Material F,” “Compound I mesylate Material G,” “Compound I napsylate Material A,” “Compound I oxalate (disordered),” “Compound I phosphate Form I,” “Compound I phosphate Form II,” “Compound I phosphate Form III,” “Compound I phosphate Form IV,” “Compound I phosphate Form V,” “Compound I sulfate Material A,” “Compound I sulfate Material B,” “Compound I sulfate Material C,” “Compound I tartrate Material A,” “Compound I tartrate Material B,” “Compound I tosylate Form I,” “Compound I tosylate Material A,” “Compound I tosylate Material C,” and “Compound I xinafoate Form I.”

Other forms of Compound I are further described herein, such as amorphous forms of Compound I, and particularly amorphous forms of phosphate complexes of Compound I.

### Definitions

As used in the present specification, the following words and phrases are generally intended to have the meanings as set forth below, except to the extent that the context in which they are used indicates otherwise.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X”. With reference to differential scanning calorimetry, the term “about” includes, in certain embodiments, the indicated amount±4° C., e.g. +2° C., e.g. +1° C.

Recitation of numeric ranges of values throughout the disclosure is intended to serve as a shorthand notation of referring individually to each separate value falling within the range inclusive of the values defining the range, and each separate value is incorporated in the specification as it were individually recited herein.

“Amido” refers to both a “C-amido” group which refers to the group —C(═O)NR^{y}R^{z} and an “N-amido” group which refers to the group —NR^{y}C(═O)R^{z}, wherein R^{y} and R^{z} are independently selected from the group consisting of hydrogen, alkyl, aryl, heteralkyl, heteroaryl (each of which may be optionally substituted), and where R^{y} and R^{z} are optionally joined together with the nitrogen or carbon bound thereto to form an optionally substituted heterocycloalkyl.

“Amino” refers to the group —NR^{y}R^{z} wherein R^{y} and R^{z} are independently selected from the group consisting of hydrogen, alkyl, aryl, heteralkyl, heteroaryl (each of which may be optionally substituted), and where R^{y} and R^{z} are optionally joined together with the nitrogen bound thereto to form a heterocycloalkyl or heteroaryl heteroaryl (each of which may be optionally substituted).

“Amidino” refers to the group —C(═NR^{x})NR^{y}R^{z} where R^{x}, R^{y}, and R^{z} are independently selected from the group consisting of hydrogen, alkyl, aryl, heteralkyl, heteroaryl (each of which may be optionally substituted), and where R^{y} and R^{z} are optionally joined together with the nitrogen bound thereto to form a heterocycloalkyl or heteroaryl (each of which may be optionally substituted).

“Alkyl” refers to an unbranched or branched saturated hydrocarbon chain. As used herein, alkyl has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e. —CH(CH₃)₂).

“Alkenyl” refers to an alkyl group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkynyl” refers to an alkyl group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Aryl” refers to an aromatic carbocyclic group having a single ring (e.g. monocyclic) or multiple rings (e.g. bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include phenyl, naphthyl, fluorenyl, and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e. the cyclic group having at least one double bond). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl.

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR—, —O—, —S—, —S(O)—, —S(O)₂—, and the like, where R is H, alkyl, aryl, cycloalkyl, heteroalkyl, heteroaryl or heterocyclyl, each of which may be optionally substituted. Examples of heteroalkyl groups include —OCH₃, —CH₂OCH₃, —SCH₃, —CH₂SCH₃, —NRCH³, and —CH₂NRCH₃, where R is hydrogen, alkyl, aryl, arylalkyl, heteroalkyl, or heteroaryl, each of which may be optionally substituted. As used herein, heteroalkyl include 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings, or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl); and 1 to 5 heteroatoms, 1 to 4 heteroatoms, 1 to 3 ring heteroatoms, 1 to 2 ring heteroatoms, or 1 ring heteroatom independently selected from nitrogen, oxygen, and sulfur. Examples of heteroaryl groups include pyrimidinyl, purinyl, pyridyl, pyridazinyl, benzothiazolyl, and pyrazolyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. Examples of heterocyclyl groups include pyrrolidinyl, piperidinyl, piperazinyl, oxetanyl, dioxolanyl, azetidinyl, and morpholinyl. As used herein, the term “bridged-heterocyclyl” refers to a four- to ten-membered cyclic moiety connected at two non-adjacent atoms of the heterocyclyl with one or more (e.g., 1 or 2) four- to ten-membered cyclic moiety having at least one heteroatom where each heteroatom is independently selected from nitrogen, oxygen, and sulfur. As used herein, bridged-heterocyclyl includes bicyclic and tricyclic ring systems. Also used herein, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. Examples of the spiro-heterocyclyl rings include bicyclic and tricyclic ring systems, such as 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl, the last mentioned group contains the atom by which the moiety is attached to the rest of the molecule.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Forms of Compound I or complexes, hydrates, solvates thereof are provided herein. In one embodiment, reference to a form of Compound I or a complex, hydrate, or solvate thereof means that at least 50% to 99% (e.g., at least 50%, at least 55%, at least 60%, at least 65%, at least 70%, at least 75%, at least 80%, at least 85%, at least 90%, at least 95%, or at least 99%) of Compound I or a complex, hydrate, or solvate thereof present in a composition is in the designated form. For instance, in one embodiment, reference to Compound I phosphate Form I means that at least 50%, at least 55%, at least 60%, at least 65%, at least 70%, at least 75%, at least 80%, at least 85%, at least 90%, at least 95%, or at least 99% of Compound I phosphate present in a composition is in Form I.

The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (melting point).

The term “substantially crystalline” as used herein is intended to mean that greater than 50%; or greater than 55%; or greater than 60%; or greater than 65%; or greater than 70%; or greater than 75%; or greater than 80%; or greater than 85%; or greater than 90%; or greater than 95%, or greater than 99% of the compound present in a composition is in crystalline form. “Substantially crystalline” can also refer to material which has no more than about 20%, or no more than about 10%, or no more than about 5%, or no more than about 2% in the amorphous form. Likewise, the term “substantially” when qualifying any form of a compound described herein is intended to mean that greater than 50%; or greater than 55%; or greater than 60%; or greater than 65%; or greater than 70%; or greater than 75%; or greater than 80%; or greater than 85%; or greater than 90%; or greater than 95%, or greater than 99% of the compound is present in the designated form.

The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

The term “complex” refers to a formation resulting from the interaction between Compound I and another component (e.g., a molecule, atom, or ion). In some embodiments, a complex may refer to a salt or co-crystal of Compound I.

The term “solvate” refers to a complex formed by combining Compound I, or a salt or co-crystal thereof, and a solvent. As used herein, the term “solvate” includes a hydrate (i.e., a solvate when the solvent is water).

The term “desolvated” refers to a Compound I form that is a solvate as described herein, and from which solvent molecules have been partially or completely removed. Desolvation techniques to produce desolvated forms include, without limitation, exposure of a Compound I form (solvate) to a vacuum, subjecting the solvate to elevated temperature, exposing the solvate to a stream of gas, such as air or nitrogen, or any combination thereof. Thus, a desolvated Compound I form can be anhydrous, i.e., completely without solvent molecules, or partially solvated wherein solvent molecules are present in stoichiometric or non-stoichiometric amounts.

The term “co-crystal” refers to a molecular complex of an ionized or non-ionized form of a compound disclosed herein and one or more non-ionized co-crystal formers connected through non-covalent interactions. In some embodiments, the co-crystals disclosed herein may include a non-ionized form of Compound I (e.g., Compound I free base) and one or more non-ionized co-crystal formers, where non-ionized Compound I and the co-crystal former(s) are connected through non-covalent interactions. In some embodiments, co-crystals disclosed herein may include an ionized form of Compound I (e.g., a salt of Compound I) and one or more non-ionized co-crystals formers, where ionized Compound I and the co-crystal former(s) are connected through non-covalent interactions. Co-crystals may additionally be present in anhydrous, solvated or hydrated forms. In certain embodiments, the formation of a salt or co-crystal may depend on the difference between the pKa values of the acidic and basic components thereof. For instance, a salt may form where large pKa differences exist, thereby allowing proton transfer between the acidic and basic components thereof. Conversely, no such proton transfer occurs in a co-crystal. In certain embodiments, the co-crystal can have an improved property as compared to the free form (i.e., the free molecule, zwitter ion, hydrate, solvate, etc.) or a salt (which includes salt hydrates and solvates). In further embodiments, the improved property is selected from the group consisting of: increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like.

The term “co-crystal former” or “co-former” refers to one or more pharmaceutically acceptable bases or pharmaceutically acceptable acids disclosed herein in association with Compound I, or any other compound disclosed herein.

Any formula or structure given herein, including Compound I, is also intended to represent unlabeled forms as well as isotopically labeled forms of the compounds. Isotopically labeled compounds have structures depicted by the formulae given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which isotopes such as ³H, ¹³C and ¹⁴C are incorporated, may be prepared. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds of Formula I in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula I.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen” the position is understood to have hydrogen at its natural abundance isotopic composition. Accordingly, in the compounds of this disclosure any atom specifically designated as a deuterium (D) is meant to represent deuterium.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. The therapeutically effective amount may vary depending on the subject, and disease or condition being treated, the weight and age of the subject, the severity of the disease or condition, and the manner of administering, which can readily be determined by one or ordinary skill in the art.

As used herein “carrier” or “pharmaceutically acceptable carrier” includes excipients or agents such as solvents, diluents, dispersion media, coatings, antibacterial and antifungal agents, isotonic and absorption delaying agents and the like that are not deleterious to the compound of the invention or use thereof. The use of such carriers and agents to prepare compositions of pharmaceutically active substances is well known in the art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

As used herein, the term “modulating” or “modulate” refers to an effect of altering a biological activity, especially a biological activity associated with a particular biomolecule such as a protein kinase. For example, an agonist or antagonist of a particular biomolecule modulates the activity of that biomolecule, e.g., an enzyme, by either increasing (e.g., agonist, activator), or decreasing (e.g., antagonist, inhibitor) the activity of the biomolecule, such as an enzyme. Such activity is typically indicated in terms of an inhibitory concentration (IC₅₀) or excitation concentration (EC₅₀) of the compound for an inhibitor or activator, respectively, with respect to, for example, an enzyme.

In addition, abbreviations as used herein have respective meanings as follows:

| | |
|---|---|
| μL | Microliter |
| μm | Micrometer |
| 2-MeTHF | 2-Methyl tetrahydrofuran |
| API | Active pharmaceutical ingredient |
| BET | Bromodomain and extraterminal |
| BuOAc | Butyl acetate |
| DCM | Dichloromethane |
| DMAc | Dimethylacetamide |
| DMF | Dimethylformamide |
| DMSO | Dimethyl sulfoxide |
| DSC | Differential scanning calorimetry |
| DVS | Dynamic vapor sorption |
| eq. | Equivalents |
| EtOAc | Ethyl acetate |
| EtOH | Ethanol |
| g | Gram |
| h | Hour |
| HCl | Hydrochloride |
| HOAc | Acetic acid |
| IPA | Isopropanol |
| IPAc | Isopropyl acetate |
| IPE | Diisopropyl ether |
| KF | Karl Fischer titration |
| LC/MS | Liquid chromatography-mass spectrometry |
| MeCN | Acetonitrile |
| MEK | Methyl ethyl ketone |
| MeOH | Methanol |
| MIBK | Methyl iso-butyl ketone |
| mg | Milligram |
| min | Minute |
| mL/ml | Milliliter |
| MTBE | Methyl tert-butyl ether |
| NMP | N-Methyl-2-pyrrolidone |
| NMR | Nuclear magnetic resonance |
| PLM | Polarized light microscopy |
| RH | Relative humidity |
| RT | Room temperature |
| s | Second |
| TFE | Trifluoroethanol |
| TGA | Thermogravimetric analysis |
| THF | Tetrahydrofuran |
| v/v | Volume to volume |
| wt | Weight |
| w/w | Weight to weight |
| XRPD | X-ray powder diffraction |

### Forms of Compound I

As described generally above, the present disclosure provides crystalline forms of Compound I and Compound I complexes (e.g., salts or co-crystals), hydrates or solvates thereof. Additional forms (including amorphous forms) are also discussed further herein. It is of note that the crystalline forms of Compound I (free base), the crystalline forms of Compound I complexes (e.g., salts or co-crystals), hydrates or solvates thereof, and other forms (e.g., amorphous or disordered forms) of Compound I (free base) and Compound I complexes, hydrates, or solvates thereof are collectively referred to herein as “forms of Compound I.”

Compound I Form I

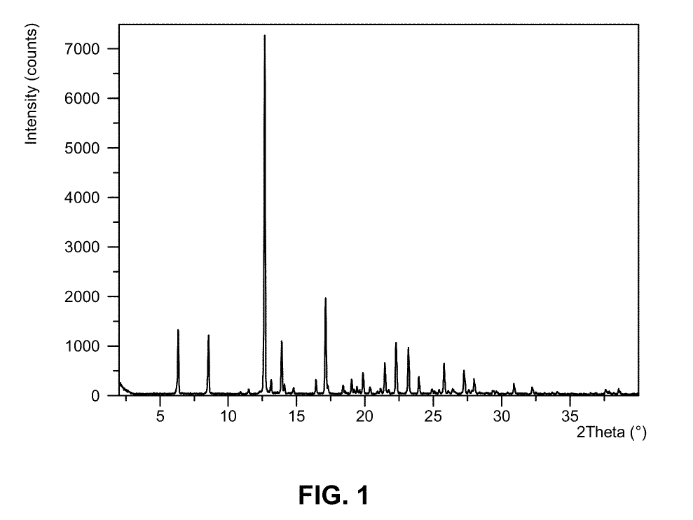

The present disclosure provides, in one embodiment, a crystalline form of (2-cyclopropyl-6-(3,5-dimethylisoxazol-4-yl)-1H-benzo[d]imidazol-4-yl)di(pyridin-2-yl)methanol (Compound I Form I) characterized by an X-ray powder diffractogram comprising the following peaks: 8.6, 12.7, and 17.1° 2θ±0.2° 2θ, as determined on a diffractometer using Cu-Kα radiation at a wavelength of 1.5406 Å. In one embodiment, the diffractogram of Compound I Form I further comprises one or more peaks at: 6.4, 13.9, and 22.3° 2θ±0.2° 2θ. In one embodiment, the diffractogram of Compound I Form I comprises at least two of the following peaks: 6.4, 8.6, 12.7, 13.9, 17.1, 19.9, 21.4, 22.3, 23.2, 23.9, 25.8, and 27.2° 2θ±0.2° 2θ. In one embodiment, the diffractogram of Compound I Form I comprises at least four of the following peaks: 6.4, 8.6, 12.7, 13.9, 17.1, 19.9, 21.4, 22.3, 23.2, 23.9, 25.8, and 27.2° 2θ±0.2° 2θ. In one embodiment, the diffractogram of Compound I Form I comprises at least six of the following peaks: 6.4, 8.6, 12.7, 13.9, 17.1, 19.9, 21.4, 22.3, 23.2, 23.9, 25.8, and 27.2° 2θ±0.2° 2θ. In one embodiment, the diffractogram of Compound I Form I comprises at least eight of the following peaks: 6.4, 8.6, 12.7, 13.9, 17.1, 19.9, 21.4, 22.3, 23.2, 23.9, 25.8, and 27.2° 2θ±0.2° 2θ. In one embodiment, the diffractogram of Compound I Form I comprises each of the following peaks: 6.4, 8.6, 12.7, 13.9, 17.1, 19.9, 21.4, 22.3, 23.2, 23.9, 25.8, and 27.2° 2θ±0.2° 2θ. In one embodiment, Compound I Form I is characterized by the X-ray powder diffractogram as substantially shown in FIG. 1.

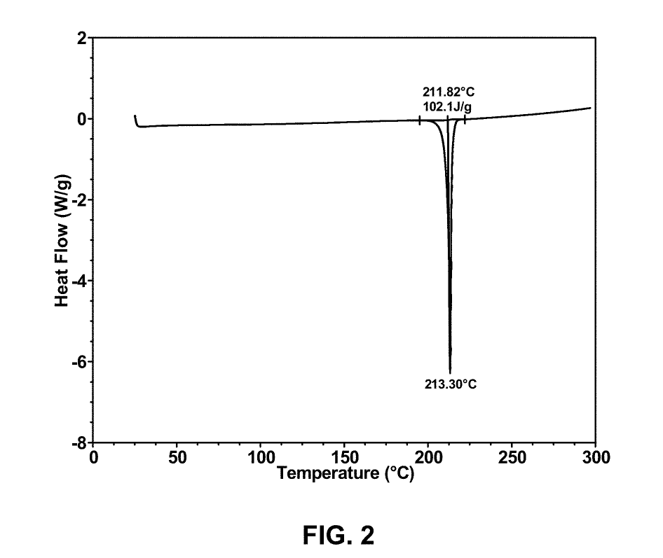

In one embodiment, Compound I Form I is characterized by a differential scanning calorimetry (DSC) curve that comprises an endotherm with onset at about 212° C. In one embodiment, Compound I Form I is characterized by the DSC curve as substantially shown in FIG. 2.

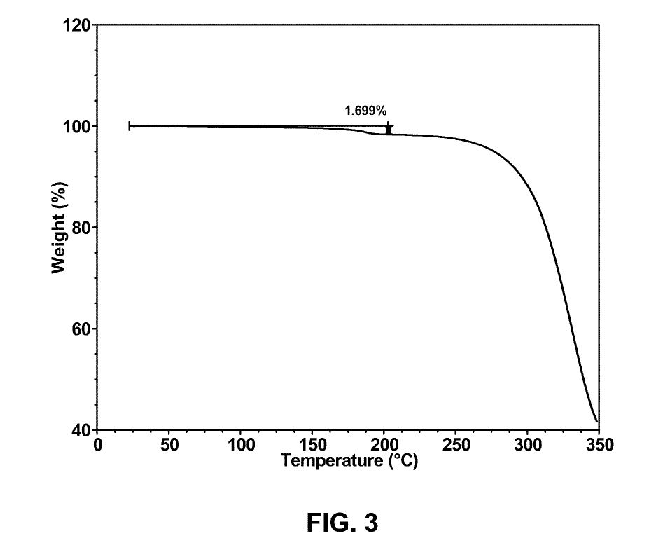

In one embodiment, Compound I Form I is characterized by a thermogravimetric analysis (TGA) thermogram showing a weight loss of about 1.7% from about 150° C. to about 200° C. In one embodiment, Compound I Form I is characterized by the TGA thermogram as substantially shown in FIG. 3.

In one embodiment, Compound I Form I is characterized as anhydrous, comprising substantially no water as measured by Karl Fischer (KF) analysis.

The present disclosure also provides at least one process for making Compound I Form I. In one embodiment, the process includes obtaining Compound I Form I from a solvent or solvent mixture selected from the group selected from: acetone/water, heptane/acetone, heptane/dichloromethane (DCM), heptane/ethanol (EtOH), acetonitrile (MeCN), butyl acetate (BuOAc), dichloromethane (DCM), dimethylformamide (DMF)/methyl tert-butyl ether (MTBE), ethanol (EtOH), isopropanol (IPA), EtOAc, isopropyl acetate (IPAc), methanol (MeOH), buanol, methyl ethyl ketone (MEK), methyl iso-butyl ketone (MIBK), tetrahydrofuran (THF), 2-methyltetrahydrofuran (2-MeTHF), N-methyl-2-pyrrolidone (NMP)/diisopropyl ether (IPE), toluene, and trifluoroethanol (TFE) using slurries, evaporation, cooling, lyophilization, and/or precipitation with anti-solvents. In one exemplary embodiment, the process includes contacting Compound I with pyridine, THF, water, and EtOAc, whereby Compound I Form I is formed. In one embodiment, the process for making Compound I Form I is as described in the Examples provided herein.

Compound I Form II

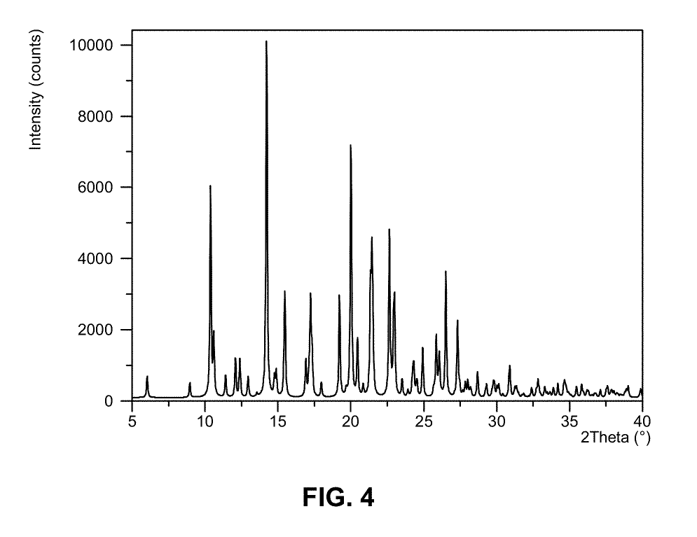

The present disclosure provides, in one embodiment, a crystalline form of (2-cyclopropyl-6-(3,5-dimethylisoxazol-4-yl)-1H-benzo[d]imidazol-4-yl)di(pyridin-2-yl)methanol (Compound I Form II) characterized by an X-ray powder diffractogram comprising the following peaks: 10.4, 14.2, and 20.0° 2θ±0.2° 2θ, as determined on a diffractometer using Cu-Kα radiation at a wavelength of 1.5406 Å. In one embodiment, the diffractogram of Compound I Form II further comprises one or more peaks at 21.5 and 26.5° 2θ±0.2° 2θ. In one embodiment, the diffractogram of Compound I Form II comprises at least two of the following peaks: 12.1, 12.4, 14.2, 10.4, 10.6, 15.5, 16.9, 17.2, 19.2, 20.0, 20.5, 21.3, 21.5, 22.6, 23.0, 24.3, 24.9, 25.9, 26.1, 26.5, 27.3, and 30.9° 2θ±0.2° 2θ. In one embodiment, the diffractogram of Compound I Form II comprises at least four of the following peaks: 12.1, 12.4, 14.2, 10.4, 10.6, 15.5, 16.9, 17.2, 19.2, 20.0, 20.5, 21.3, 21.5, 22.6, 23.0, 24.3, 24.9, 25.9, 26.1, 26.5, 27.3, and 30.9° 2θ±0.2° 2θ. In one embodiment, the diffractogram of Compound I Form II comprises at least six of the following peaks: 12.1, 12.4, 14.2, 10.4, 10.6, 15.5, 16.9, 17.2, 19.2, 20.0, 20.5, 21.3, 21.5, 22.6, 23.0, 24.3, 24.9, 25.9, 26.1, 26.5, 27.3, and 30.9° 2θ±0.2° 2θ. In one embodiment, the diffractogram of Compound I Form II comprises at least eight of the following peaks: 12.1, 12.4, 14.2, 10.4, 10.6, 15.5, 16.9, 17.2, 19.2, 20.0, 20.5, 21.3, 21.5, 22.6, 23.0, 24.3, 24.9, 25.9, 26.1, 26.5, 27.3, and 30.9° 2θ±0.2° 2θ. In one embodiment, the diffractogram of Compound I Form II comprises each of the following peaks: 12.1, 12.4, 14.2, 10.4, 10.6, 15.5, 16.9, 17.2, 19.2, 20.0, 20.5, 21.3, 21.5, 22.6, 23.0, 24.3, 24.9, 25.9, 26.1, 26.5, 27.3, and 30.9° 2θ±0.2° 2θ. In one embodiment, Compound I Form II is characterized by the X-ray powder diffractogram as substantially shown in FIG. 4.

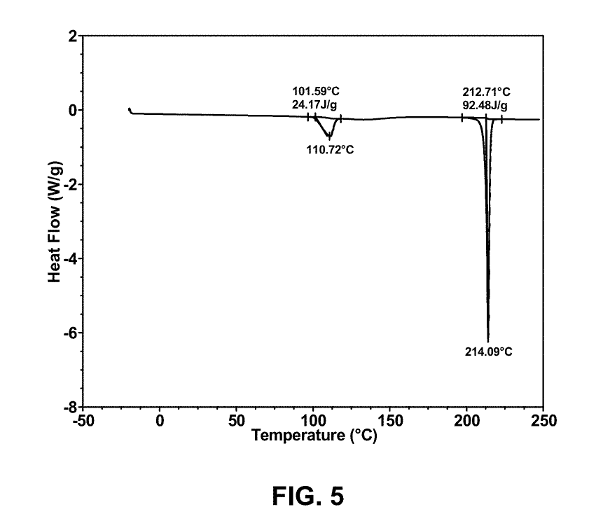

In one embodiment, Compound I Form II is characterized by a differential scanning calorimeter (DSC) curve that comprises an endotherm with onset at about 213° C. In one embodiment, the DSC curve of Compound I Form II comprises an additional endotherm with onset at about 102° C. In one embodiment, Compound I Form II is characterized by the DSC curve as substantially shown in FIG. 5.

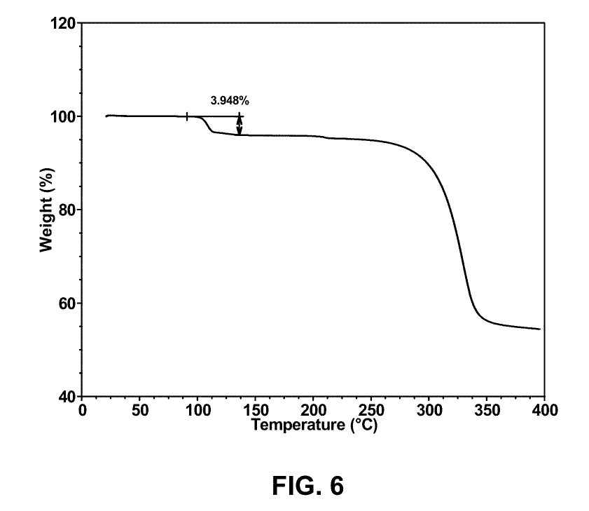

In one embodiment, Compound I Form II is characterized by a thermogravimetric analysis (TGA) thermogram showing a weight loss of about 3.9% from about 90° C. to about 110° C. In one embodiment, Compound I Form II is characterized by the TGA thermogram as substantially shown in FIG. 6.

The present disclosure also provides at least one process for making Compound I Form II. In one embodiment, the process comprises the step of evaporating Compound I from a solvent mixture of IPA and EtOH, whereby Compound I Form II is formed. In one embodiment, the ratio of IPA to EtOH is about 5:1. In one embodiment, the process for making Compound I Form I is as described in the Examples provided herein.

Compound I Material A

The present disclosure provides, in one embodiment, a crystalline form of (2-cyclopropyl-6-(3,5-dimethylisoxazol-4-yl)-1H-benzo[d]imidazol-4-yl)di(pyridin-2-yl)methanol (Compound I Material A) characterized by an X-ray powder diffractogram comprising the following peaks: 8.0, 10.2, and 16.1° 2θ±0.2° 2θ, as determined on a diffractometer using Cu-Kα radiation at a wavelength of 1.5406 Å. In one embodiment, the diffractogram of Compound I Material A further comprises one or more peaks at: 8.7, 10.4, 13.7, 17.8, and 22.0° 2θ±0.2° 2θ. In one embodiment, the diffractogram of Compound I Material A comprises at least two of the following peaks: 8.0, 10.2, 16.1, 17.8, and 22.0° 2θ±0.2° 2θ. In one embodiment, the diffractogram of Compound I Material A comprises at least four of the following peaks: 8.0, 10.2, 16.1, 17.8, and 22.0° 2θ±0.2° 2θ. In one embodiment, the diffractogram of Compound I Material A comprises each of the following peaks: 8.0, 10.2, 16.1, 17.8, and 22.0° 2θ±0.2° 2θ.

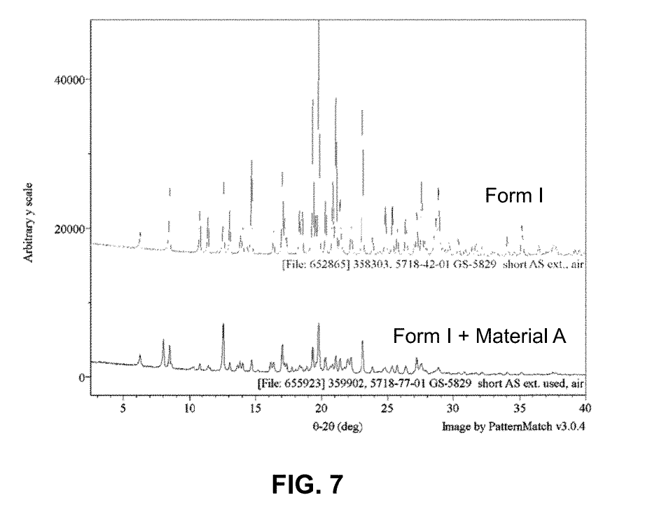

In one embodiment, Compound I Material A is present as a mixture with Compound I Form I. In one embodiment, Compound I Material A is characterized by the X-ray powder diffractogram as substantially shown in FIG. 7, which includes the presence of Compound I Form I. FIG. 7 also includes the X-ray powder diffractogram of Compound I Form I for comparison.

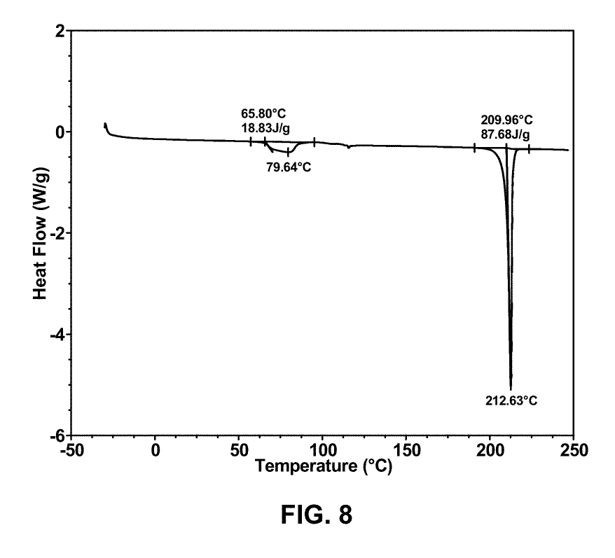

In one embodiment, Compound I Material A, present as a mixture with Compound I Form I, is characterized by a differential scanning calorimeter (DSC) curve that comprises an endotherm with onset at about 210° C. In one embodiment, the DSC curve of Compound I Material A, as a mixture with Compound I Form I, comprises an additional endotherm with onset at about 66° C. In one embodiment, Compound I Material A, as a mixture with Compound I Form I, is characterized by the DSC curve as substantially shown in FIG. 8.

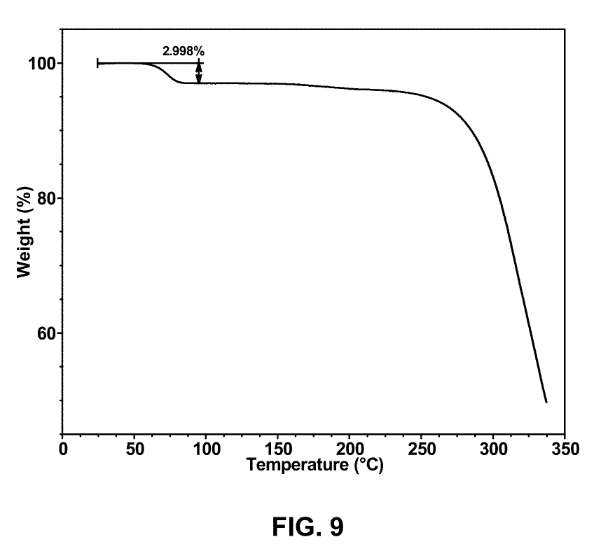

In one embodiment, Compound I Material A, present as a mixture with Compound I Form I. is characterized by a thermogravimetric analysis (TGA) thermogram showing a weight loss of about 3% below about 100° C. In one embodiment, Compound I Material A, as a mixture with Compound I Form I, is characterized by the TGA thermogram as substantially shown in FIG. 9.

In one embodiment, Compound I Material A is characterized as ap-dioxane solvate. In one embodiment, Compound I Material A is characterized as comprising minimal water as measured by KF analysis.

The present disclosure also provides at least one process for making Compound I Material A. In one embodiment, the process comprises lyophilizing a solution comprising Compound I and dioxane, whereby Compound I Material A is formed as a mixture with Compound I Form I. In one embodiment, the process for making Compound I Material A is as described in the Examples provided herein.

Compound I Amorphous

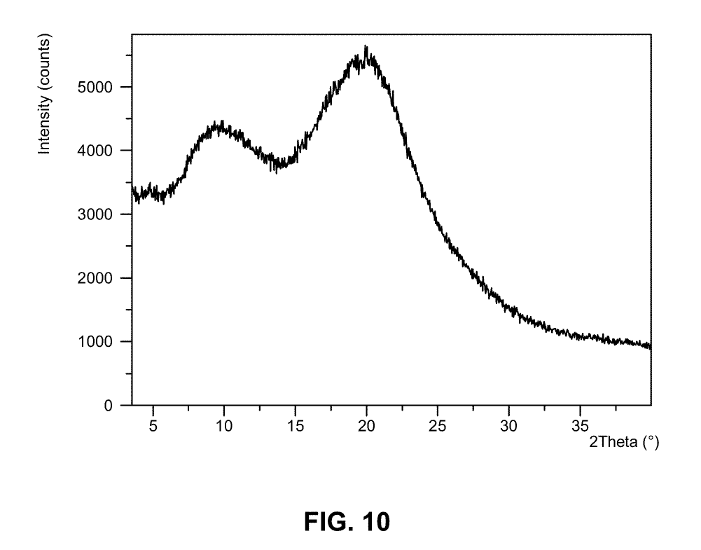

The present disclosure provides, in one embodiment, an amorphous form of (2-cyclopropyl-6-(3,5-dimethylisoxazol-4-yl)-1H-benzo[d]imidazol-4-yl)di(pyridin-2-yl)methanol (Compound I amorphous). In one embodiment, Compound I amorphous is characterized by the X-ray powder diffractogram as substantially shown in FIG. 10.

The present disclosure also provides at least one process for making Compound I amorphous. In one embodiment, the process comprises the step of evaporating Compound I from a solvent comprising TFE, whereby Compound I amorphous is formed. In one embodiment, the process for making Compound I amorphous is as described in the Examples provided herein.

Compound I Phosphate Form I

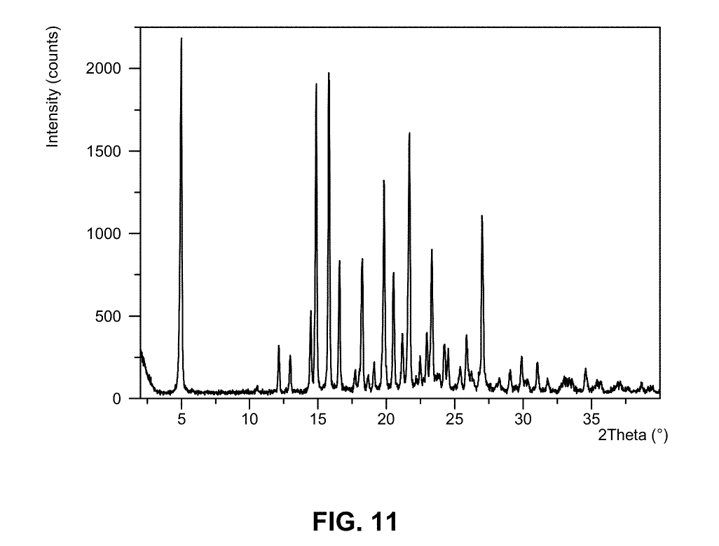

The present disclosure provides, in one embodiment, a phosphate complex of (2-cyclopropyl-6-(3,5-dimethylisoxazol-4-yl)-1H-benzo[d]imidazol-4-yl)di(pyridin-2-yl)methanol having a crystalline form (Compound I phosphate Form I). In one embodiment, Compound I phosphate Form I corresponds to a phosphate salt of Compound I. In one embodiment, Compound I phosphate Form I corresponds to a phosphate co-crystal of Compound I. Compound I phosphate Form I is characterized by an X-ray powder diffractogram comprising the following peaks: 5.0, 15.8, and 21.7° 2θ±0.2° 2θ, as determined on a diffractometer using Cu-Kα radiation at a wavelength of 1.5406 Å. In one embodiment, the diffractogram of Compound I phosphate Form I further comprises one or more peaks at: 12.1, 13.0, 14.9, 19.8, 23.3, and 27.0° 2θ±0.2° 2θ. In one embodiment, the diffractogram of Compound I phosphate Form I comprises at least two of the following peaks: 5.0, 12.1, 13.0, 14.5, 14.9, 15.8, 16.6, 18.2, 19.8, 20.5, 21.2, 21.7, 22.9, 23.3, 24.2, 24.5, 25.9, 27.0, and 29.9° 2θ±0.2° 2θ. In one embodiment, the diffractogram of Compound I phosphate Form I comprises at least four of the following peaks: 5.0, 12.1, 13.0, 14.5, 14.9, 15.8, 16.6, 18.2, 19.8, 20.5, 21.2, 21.7, 22.9, 23.3, 24.2, 24.5, 25.9, 27.0, and 29.9° 2θ±0.2° 2θ. In one embodiment, the diffractogram of Compound I phosphate Form I comprises at least six of the following peaks: 5.0, 12.1, 13.0, 14.5, 14.9, 15.8, 16.6, 18.2, 19.8, 20.5, 21.2, 21.7, 22.9, 23.3, 24.2, 24.5, 25.9, 27.0, and 29.9° 2θ±0.2° 2θ. In one embodiment, the diffractogram of Compound I phosphate Form I comprises at least eight of the following peaks: 5.0, 12.1, 13.0, 14.5, 14.9, 15.8, 16.6, 18.2, 19.8, 20.5, 21.2, 21.7, 22.9, 23.3, 24.2, 24.5, 25.9, 27.0, and 29.9° 2θ±0.2° 2θ. In one embodiment, the diffractogram of Compound I phosphate Form I comprises each of the following peaks: 5.0, 12.1, 13.0, 14.5, 14.9, 15.8, 16.6, 18.2, 19.8, 20.5, 21.2, 21.7, 22.9, 23.3, 24.2, 24.5, 25.9, 27.0, and 29.9° 2θ±0.2° 2θ. In one embodiment, Compound I phosphate Form I is characterized by the X-ray powder diffractogram as substantially shown in FIG. 11.

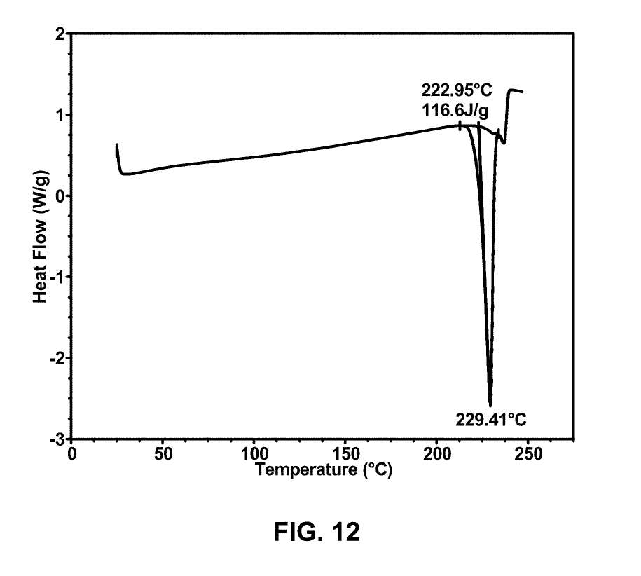

In one embodiment, Compound I phosphate Form I is characterized by a differential scanning calorimeter (DSC) curve that comprises an endotherm with onset at about 223° C. In one embodiment, Compound I phosphate Form I is characterized by a differential scanning calorimeter (DSC) curve that comprises an endotherm with onset at 223° C.±4° C. In one embodiment, Compound I phosphate Form I is characterized by a differential scanning calorimeter (DSC) curve that comprises an endotherm with onset at 223° C.±2° C. In one embodiment, Compound I phosphate Form I is characterized by a differential scanning calorimeter (DSC) curve that comprises an endotherm with onset at 223° C.±1° C. In one embodiment, Compound I phosphate Form I is characterized by the DSC curve as substantially shown in FIG. 12.

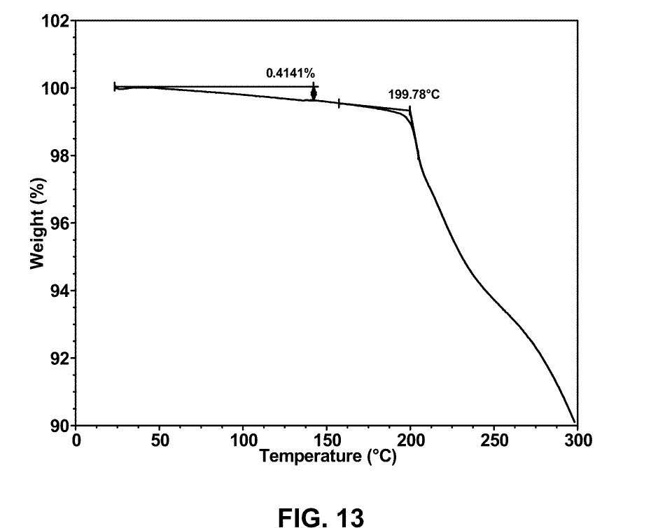

In one embodiment, Compound I phosphate Form I is characterized by a thermogravimetric analysis (TGA) thermogram showing a weight loss of about 0.4% below about 150° C. In one embodiment, Compound I phosphate Form I is characterized by the TGA thermogram as substantially shown in FIG. 13.

In one embodiment, Compound I phosphate Form I is characterized as anhydrous, comprising substantially no water as measured by KF analysis. In one embodiment, Compound I phosphate Form I is characterized by a 1:1 ratio of Compound I to phosphoric acid as determined by an ion chromatography analysis. In one embodiment, Compound I phosphate Form I has a kinetic aqueous solubility of about 3 mg/mL.

The present disclosure also provides at least one process for making Compound I phosphate Form I. In one embodiment, the process comprises contacting Compound I with phosphoric acid and a solvent, whereby Compound I phosphate From I formed. In one embodiment, the solvent is selected from the group consisting of: MeOH, EtOH, IPA, water, DCM, DMF, EtOAc, MIBK, MEK, THF, 2-MeTHF, IPAc, MTBE, toluene, heptane, acetonitrile, and combinations thereof. In one embodiment, the process for making Compound I phosphate Form I is as described in the Examples provided herein.

Compound I Phosphate Form II

The present disclosure provides, in one embodiment, a phosphate complex of (2-cyclopropyl-6-(3,5-dimethylisoxazol-4-yl)-1H-benzo[d]imidazol-4-yl)di(pyridin-2-yl)methanol having a crystalline form (Compound I phosphate Form II). In one embodiment, Compound I phosphate Form II corresponds to a phosphate salt of Compound I. In one embodiment, Compound I phosphate Form II corresponds to a phosphate co-crystal of Compound I.

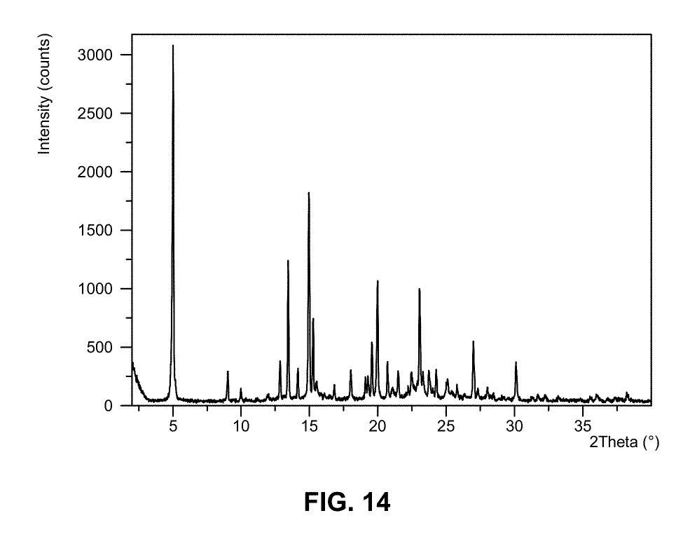

Compound I phosphate Form II is characterized by an X-ray powder diffractogram comprising the following peaks: 5.0, 9.0, and 14.1° 2θ±0.2° 2θ, as determined on a diffractometer using Cu-Kα radiation at a wavelength of 1.5406 Å. In one embodiment, the diffractogram of Compound I phosphate Form II further comprises one or more peaks at: 13.4, 15.0, 15.3, 19.6, 20.0 and 23.0° 2θ±0.2° 2θ. In one embodiment, the diffractogram of Compound I phosphate Form II comprises at least two of the following peaks: 5.0, 9.0, 10.0, 12.9, 13.4, 14.1, 15.0, 15.3, 18.0, 19.6, 20.0, 20.7, 21.5, 23.0, 24.2, 27.0, and 30.1° 2θ±0.2° 2θ. In one embodiment, the diffractogram of Compound I phosphate Form II comprises at least four of the following peaks: 5.0, 9.0, 10.0, 12.9, 13.4, 14.1, 15.0, 15.3, 18.0, 19.6, 20.0, 20.7, 21.5, 23.0, 24.2, 27.0, and 30.1° 2θ±0.2° 2θ. In one embodiment, the diffractogram of Compound I phosphate Form II comprises at least six of the following peaks: 5.0, 9.0, 10.0, 12.9, 13.4, 14.1, 15.0, 15.3, 18.0, 19.6, 20.0, 20.7, 21.5, 23.0, 24.2, 27.0, and 30.1° 2θ±0.2° 2θ. In one embodiment, the diffractogram of Compound I phosphate Form II comprises at least eight of the following peaks: 5.0, 9.0, 10.0, 12.9, 13.4, 14.1, 15.0, 15.3, 18.0, 19.6, 20.0, 20.7, 21.5, 23.0, 24.2, 27.0, and 30.1° 2θ±0.2° 2θ. In one embodiment, the diffractogram of Compound I phosphate Form II comprises each of the following peaks: 5.0, 9.0, 10.0, 12.9, 13.4, 14.1, 15.0, 15.3, 18.0, 19.6, 20.0, 20.7, 21.5, 23.0, 24.2, 27.0, and 30.1° 2θ±0.2° 2θ. In one embodiment, Compound I phosphate Form II is characterized by the X-ray powder diffractogram as substantially shown in FIG. 14.

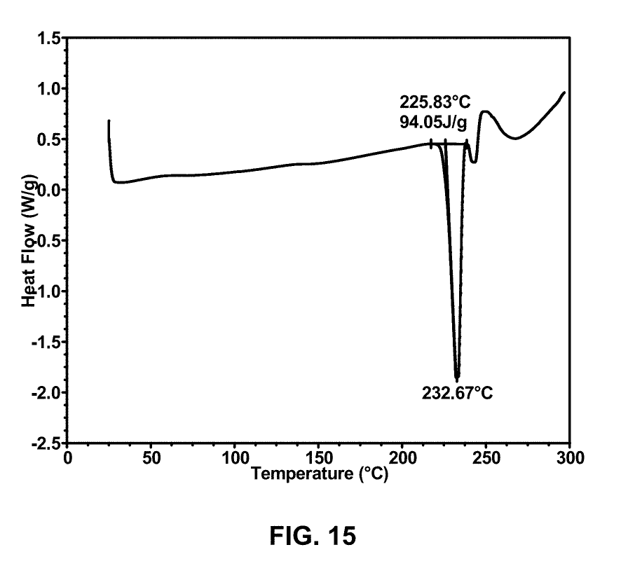

In one embodiment, Compound I phosphate Form II is characterized by a differential scanning calorimeter (DSC) curve that comprises an endotherm with onset at about 226° C. In one embodiment, Compound I phosphate Form II is characterized by the DSC curve as substantially shown in FIG. 15.

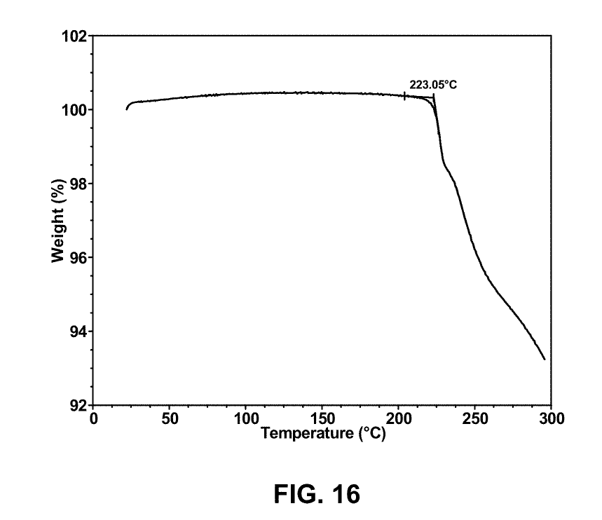

In one embodiment, Compound I phosphate Form II is characterized by thermogravimetric analysis (TGA) thermogram showing substantially no weight loss prior to the decomposition temperature thereof at about 223° C. In one embodiment, Compound I phosphate Form II is characterized by the TGA thermogram as substantially shown in FIG. 16.

In one embodiment, Compound I phosphate Form II is characterized by dynamic vapor sorption (DVS) analysis showing a water uptake from about 2.5% to about 3% at 90% RH. In one embodiment, Compound I phosphate Form II is characterized as anhydrous.

The present disclosure also provides at least one process for making Compound I phosphate Form II. In one embodiment, the process comprises contacting Compound I with MeOH, IPA and phosphoric acid, whereby Compound I phosphate From II formed. In one embodiment, the ratio of MeOH to IPA is about 1:1. In one embodiment, the process for making Compound I phosphate Form II is as described in the Examples provided herein.

Compound I Phosphate Form III

The present disclosure provides, in one embodiment, a phosphate complex of (2-cyclopropyl-6-(3,5-dimethylisoxazol-4-yl)-1H-benzo[d]imidazol-4-yl)di(pyridin-2-yl)methanol having a crystalline form (Compound I phosphate Form III). In one embodiment, Compound I phosphate Form III corresponds to a phosphate salt of Compound I. In one embodiment, Compound I phosphate Form III corresponds to a phosphate co-crystal of Compound I.

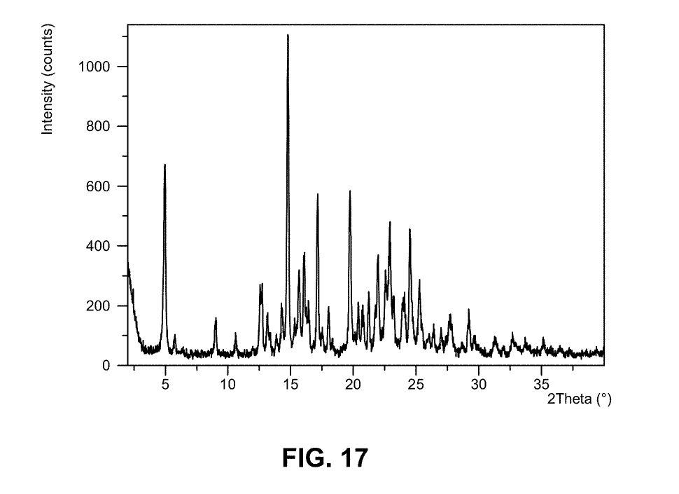

Compound I phosphate Form III is characterized by an X-ray powder diffractogram comprising the following peaks: 14.8, 19.7, and 24.5° 2θ±0.2° 2θ, as determined on a diffractometer using Cu-Kα radiation at a wavelength of 1.5406 Å. In one embodiment, the diffractogram of Compound I phosphate Form III further comprises one or more peaks at: 5.0, 5.8, 12.7, 15.7, 16.1, 17.1, 21.9, and 22.9° 2θ±0.2° 2θ. In one embodiment, the diffractogram of Compound I phosphate Form III comprises at least two of the following peaks: 5.0, 5.8, 9.0, 12.5, 12.7, 13.1, 14.3, 14.8, 15.7, 16.1, 16.4, 17.1, 18.0, 19.7, 20.4, 21.2, 21.9, 22.6, 22.9, 23.2, 23.9, 24.1, 24.5, 25.3, and 29.2° 2θ±0.2° 2θ. In one embodiment, the diffractogram of Compound I phosphate Form III comprises at least four of the following peaks: 5.0, 5.8, 9.0, 12.5, 12.7, 13.1, 14.3, 14.8, 15.7, 16.1, 16.4, 17.1, 18.0, 19.7, 20.4, 21.2, 21.9, 22.6, 22.9, 23.2, 23.9, 24.1, 24.5, 25.3, and 29.2° 2θ±0.2° 2θ. In one embodiment, the diffractogram of Compound I phosphate Form III comprises at least six of the following peaks: 5.0, 5.8, 9.0, 12.5, 12.7, 13.1, 14.3, 14.8, 15.7, 16.1, 16.4, 17.1, 18.0, 19.7, 20.4, 21.2, 21.9, 22.6, 22.9, 23.2, 23.9, 24.1, 24.5, 25.3, and 29.2° 2θ±0.2° 2θ. In one embodiment, the diffractogram of Compound I phosphate Form III comprises at least eight of the following peaks: 5.0, 5.8, 9.0, 12.5, 12.7, 13.1, 14.3, 14.8, 15.7, 16.1, 16.4, 17.1, 18.0, 19.7, 20.4, 21.2, 21.9, 22.6, 22.9, 23.2, 23.9, 24.1, 24.5, 25.3, and 29.2° 2θ±0.2° 2θ. In one embodiment, the diffractogram of Compound I phosphate Form III comprises each of the following peaks: 5.0, 5.8, 9.0, 12.5, 12.7, 13.1, 14.3, 14.8, 15.7, 16.1, 16.4, 17.1, 18.0, 19.7, 20.4, 21.2, 21.9, 22.6, 22.9, 23.2, 23.9, 24.1, 24.5, 25.3, and 29.2° 2θ±0.2° 2θ. In one embodiment, Compound I phosphate Form III is characterized by the X-ray powder diffractogram as substantially shown in FIG. 17.

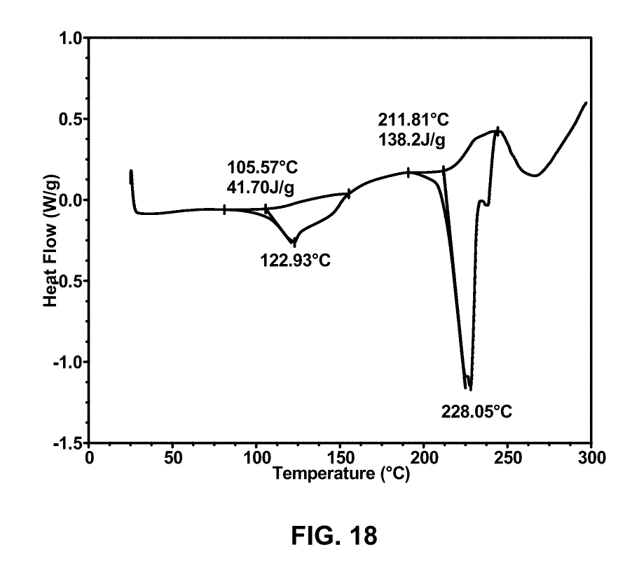

In one embodiment, Compound I phosphate Form III is characterized by a differential scanning calorimeter (DSC) curve that comprises an endotherm with onset at about 212° C. In one embodiment, the DSC curve of Compound I phosphate Form III comprises an additional endotherm with onset at about 106° C. In one embodiment, Compound I phosphate Form III is characterized by the DSC curve as substantially shown in FIG. 18.

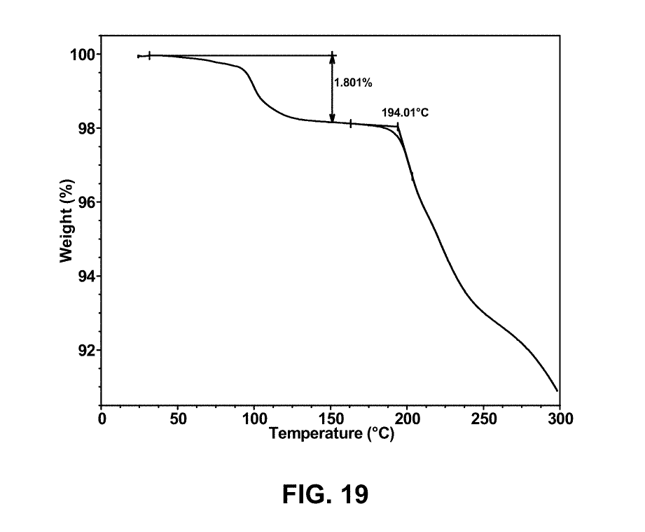

In one embodiment, Compound I phosphate Form III is characterized by a thermogravimetric analysis (TGA) thermogram showing a weight loss of about 1.8% below about 150° C. In one embodiment, Compound I phosphate Form III is characterized by the TGA thermogram as substantially shown in FIG. 19.

In one embodiment, Compound I phosphate Form III is characterized by dynamic vapor sorption (DVS) analysis showing a water uptake of about 0.7% at 90% RH. In one embodiment, Compound I phosphate Form III is characterized as a hemi-hydrate, comprising about 1.36% water, as measured by KF analysis. In one embodiment, the solubility of Compound I phosphate Form III in water is about 6 mg/mL.

The present disclosure also provides at least one process for making Compound I phosphate Form III. In one embodiment, the process comprises contacting Compound I with phosphoric acid and water, a mixture of EtOH and water, or a mixture of acetone and water, whereby Compound I phosphate Form III is formed. In one embodiment, the process for making Compound I phosphate Form III is as described in the Examples provided herein.

Compound I Phosphate Form IV

The present disclosure provides, in one embodiment, a phosphate complex of (2-cyclopropyl-6-(3,5-dimethylisoxazol-4-yl)-1H-benzo[d]imidazol-4-yl)di(pyridin-2-yl)methanol having a crystalline form (Compound I phosphate Form IV). In one embodiment, Compound I phosphate Form IV corresponds to a phosphate salt of Compound I. In one embodiment, Compound I phosphate Form IV corresponds to a phosphate co-crystal of Compound I.

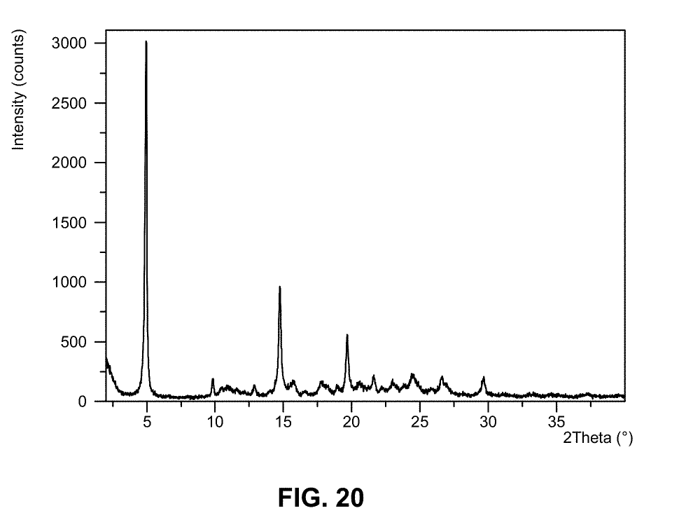

Compound I phosphate Form IV is characterized by an X-ray powder diffractogram comprising the following peaks: 9.8, 26.5, and 29.6° 2θ±0.2° 2θ, as determined on a diffractometer using Cu-Kα radiation at a wavelength of 1.5406 Å. In one embodiment, the diffractogram of Compound I phosphate Form IV further comprises one or more peaks at: 5.0, 14.7, and 19.7° 2θ±0.2° 2θ. In one embodiment, the diffractogram of Compound I phosphate Form IV comprises at least two of the following peaks: 5.0, 9.8, 12.9, 14.7, 15.8, 17.8, 19.0, 19.7, 20.5, 21.6, 23.0, 24.4, 26.5, and 29.6° 2θ±0.2° 2θ. In one embodiment, the diffractogram of Compound I phosphate Form IV comprises at least four of the following peaks: 5.0, 9.8, 12.9, 14.7, 15.8, 17.8, 19.0, 19.7, 20.5, 21.6, 23.0, 24.4, 26.5, and 29.6° 2θ±0.2° 2θ. In one embodiment, the diffractogram of Compound I phosphate Form IV comprises at least six of the following peaks: 5.0, 9.8, 12.9, 14.7, 15.8, 17.8, 19.0, 19.7, 20.5, 21.6, 23.0, 24.4, 26.5, and 29.6° 2θ±0.2° 2θ. In one embodiment, the diffractogram of Compound I phosphate Form IV comprises at least eight of the following peaks: 5.0, 9.8, 12.9, 14.7, 15.8, 17.8, 19.0, 19.7, 20.5, 21.6, 23.0, 24.4, 26.5, and 29.6° 2θ±0.2° 2θ. In one embodiment, the diffractogram of Compound I phosphate Form IV comprises each the following peaks: 5.0, 9.8, 12.9, 14.7, 15.8, 17.8, 19.0, 19.7, 20.5, 21.6, 23.0, 24.4, 26.5, and 29.6° 2θ±0.2° 2θ. In one embodiment, Compound I phosphate Form IV is characterized by the X-ray powder diffractogram as substantially shown in FIG. 20.

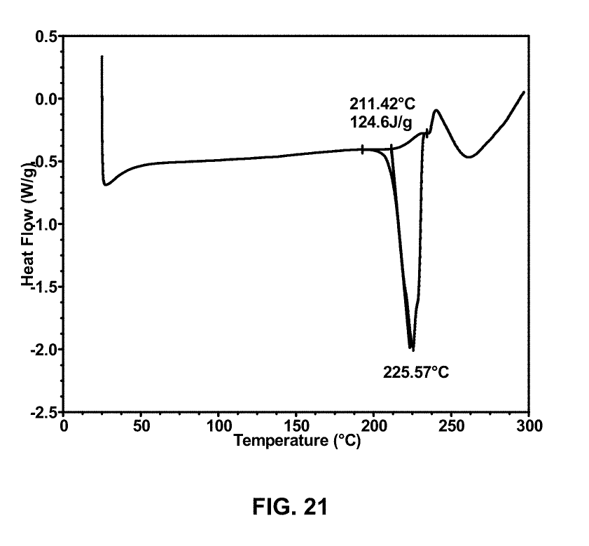

In one embodiment, Compound I phosphate Form IV is characterized by a differential scanning calorimeter (DSC) curve that comprises an endotherm with onset at about 211° C. In one embodiment, Compound I phosphate Form IV is characterized by the DSC curve as substantially shown in FIG. 21.

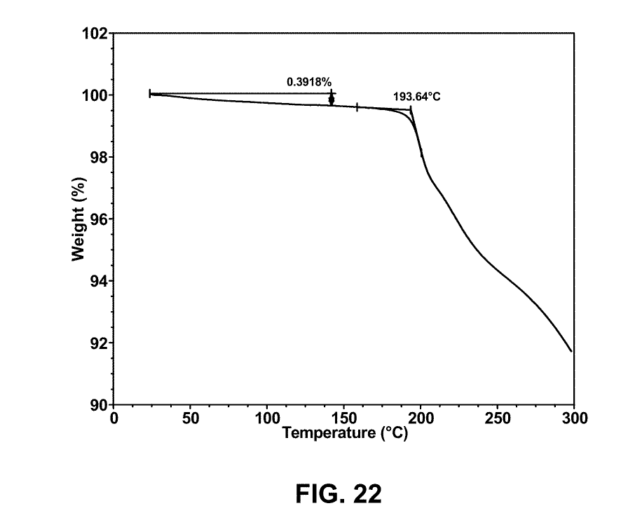

In one embodiment, Compound I phosphate Form IV is characterized by a thermogravimetric analysis (TGA) thermogram showing a weight loss of about 0.4% below about 150° C. In one embodiment, Compound I phosphate Form IV is characterized by the TGA thermogram as substantially shown in FIG. 22.

In one embodiment, Compound I phosphate Form IV comprises about 0.53% water as measured by KF analysis. In one embodiment, Compound I phosphate Form IV is characterized as substantially anhydrous or as a desolvated form of a dichloromethane (DCM) solvate of Compound I phosphate.

The present disclosure also provides at least one process for making Compound I phosphate Form IV. In one embodiment, the process comprises contacting Compound I with DCM and phosphoric acid, whereby Compound I phosphate Form IV is formed. In one embodiment, the process for making Compound I phosphate Form IV is as described in the Examples provided herein.

Compound I Phosphate Form V

The present disclosure provides, in one embodiment, a phosphate complex of (2-cyclopropyl-6-(3,5-dimethylisoxazol-4-yl)-1H-benzo[d]imidazol-4-yl)di(pyridin-2-yl)methanol having a crystalline form (Compound I phosphate Form V). In one embodiment, Compound I phosphate Form V corresponds to a phosphate salt of Compound I. In one embodiment, Compound I phosphate Form V corresponds to a phosphate co-crystal of Compound I.

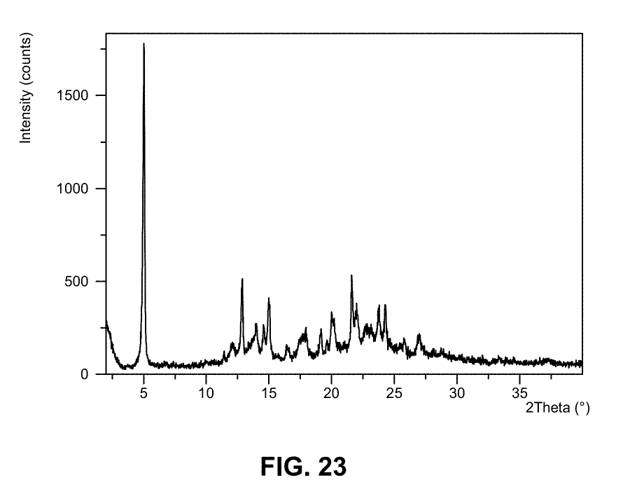

Compound I phosphate Form V is characterized by an X-ray powder diffractogram comprising the following peaks: 12.9, 14.0, and 22.0° 2θ±0.2° 2θ, as determined on a diffractometer using Cu-Kα radiation at a wavelength of 1.5406 Å. In one embodiment, the diffractogram of Compound I phosphate Form V further comprises one or more peaks at: 5.0, 14.6, 15.0, and 21.6° 2θ±0.2° 2θ. In one embodiment, the diffractogram of Compound I phosphate Form V comprises at least two, or at least four, or at least six, or at least eight, or all of the following peaks: 5.0, 12.1, 12.9, 14.0, 14.6, 15.0, 16.5, 18.0, 19.1, 20.0, 21.6, 22.0, 22.8, 23.7, 24.3, 25.8, and 26.9° 2θ±0.2° 2θ. In one embodiment, the diffractogram of Compound I phosphate Form V comprises at least four of the following peaks: 5.0, 12.1, 12.9, 14.0, 14.6, 15.0, 16.5, 18.0, 19.1, 20.0, 21.6, 22.0, 22.8, 23.7, 24.3, 25.8, and 26.9° 2θ±0.2° 2θ. In one embodiment, the diffractogram of Compound I phosphate Form V comprises at least six of the following peaks: 5.0, 12.1, 12.9, 14.0, 14.6, 15.0, 16.5, 18.0, 19.1, 20.0, 21.6, 22.0, 22.8, 23.7, 24.3, 25.8, and 26.9° 2θ±0.2° 2θ. In one embodiment, the diffractogram of Compound I phosphate Form V comprises at least eight of the following peaks: 5.0, 12.1, 12.9, 14.0, 14.6, 15.0, 16.5, 18.0, 19.1, 20.0, 21.6, 22.0, 22.8, 23.7, 24.3, 25.8, and 26.9° 2θ±0.2° 2θ. In one embodiment, the diffractogram of Compound I phosphate Form V comprises each of the following peaks: 5.0, 12.1, 12.9, 14.0, 14.6, 15.0, 16.5, 18.0, 19.1, 20.0, 21.6, 22.0, 22.8, 23.7, 24.3, 25.8, and 26.9° 2θ±0.2° 2θ. In one embodiment, Compound I phosphate Form V is characterized by the X-ray powder diffractogram as substantially shown in FIG. 23, which includes the presence of amorphous material.

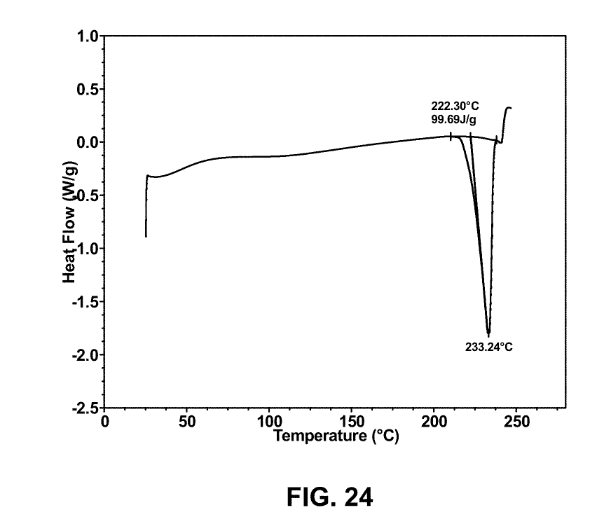

In one embodiment, Compound I phosphate Form V is characterized by a differential scanning calorimeter (DSC) curve that comprises an endotherm with onset at about 222° C. In one embodiment, the DSC curve of Compound I phosphate Form V shows an additional, broad endotherm below about 100° C. In one embodiment, Compound I phosphate Form V is characterized by the DSC curve as substantially shown in FIG. 24.

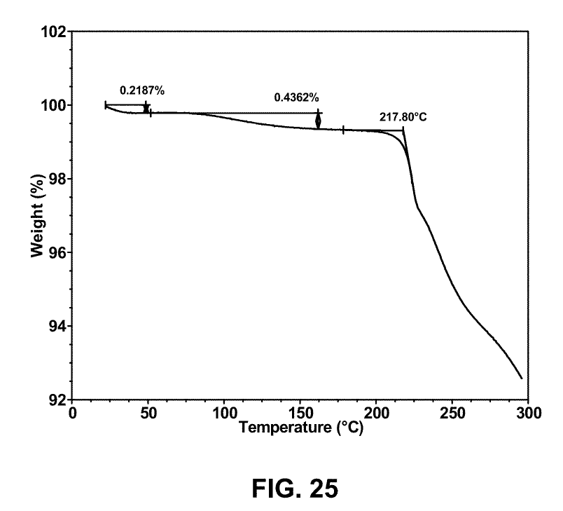

In one embodiment, Compound I phosphate Form V is characterized by a thermogravimetric analysis (TGA) thermogram showing a weight loss of about 0.2% below about 50° C. In embodiment, the TGA thermogram of Compound I phosphate Form V additionally shows a weight loss of about 0.4% from about 75° C. to about 160° C. In one embodiment, Compound I phosphate Form V is characterized by the TGA thermogram as substantially shown in FIG. 25.

In one embodiment, Compound I phosphate Form V comprises about 0.78% water as measured by KF analysis. In one embodiment, Compound I phosphate Form V is characterized as a solvated/hydrated form.

The present disclosure also provides at least one process for making Compound I phosphate Form V. In one embodiment, the process comprises contacting Compound I with MeOH, EtOAc and phosphoric acid, whereby Compound I phosphate Form V is formed. In one embodiment, the ratio of MeOH to EtOAc is about 2:10. In one embodiment, the ratio of MeOH to EtOAc is about 2:12. In one embodiment, the process for making Compound I phosphate Form V is as described in the Examples provided herein.

Compound I Phosphate (Amorphous)

The present disclosure provides, in one embodiment, a phosphate complex of (2-cyclopropyl-6-(3,5-dimethylisoxazol-4-yl)-1H-benzo[d]imidazol-4-yl)di(pyridin-2-yl)methanol having a substantially amorphous form (Compound I phosphate amorphous). In one embodiment, Compound I phosphate (amorphous) corresponds to a phosphate salt of Compound I.

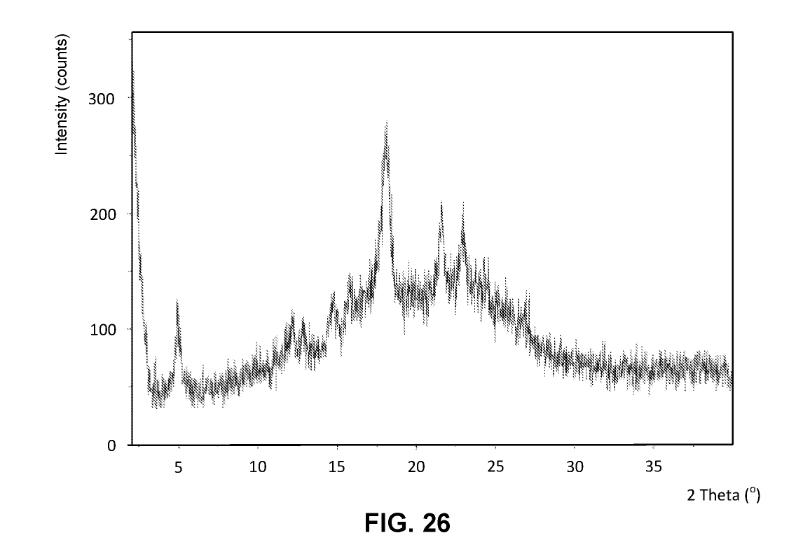

In one embodiment, Compound I phosphate amorphous is present as a mixture with a small amount of disordered Compound I phosphate material. In one embodiment, Compound I phosphate amorphous is characterized by the X-ray powder diffractogram as substantially shown in FIG. 26.

The present disclosure also provides at least one process for making Compound I phosphate amorphous. In one embodiment, the process comprises agitating Compound I phosphate Form I in heptane at RT for about several weeks, whereby Compound I phosphate amorphous is formed.

Compound I HCl Material A

The present disclosure provides, in one embodiment, a hydrochloride complex of (2-cyclopropyl-6-(3,5-dimethylisoxazol-4-yl)-1H-benzo[d]imidazol-4-yl)di(pyridin-2-yl)methanol having a crystalline form (Compound I HCl Material A). In one embodiment, Compound I HCl Material A corresponds to an HCl salt of Compound I. In one embodiment, Compound I HCl Material A corresponds to an HCl co-crystal of Compound I.

Compound I HCl Material A is characterized by an X-ray powder diffractogram comprising the following peaks: 11.0, 13.5, and 19.7° 2θ±0.2° 2θ, as determined on a diffractometer using Cu-Kα radiation at a wavelength of 1.5406 Å. In one embodiment, the diffractogram of Compound I HCl Material A further comprises one or more peaks at: 11.3, and 17.3° 2θ±0.2° 2θ. In one embodiment, the diffractogram of Compound I HCl Material A comprises at least two of the following peaks: 11.0, 11.3, 13.5, 17.3, and 19.7° 2θ±0.2° 2θ. In one embodiment, the diffractogram of Compound I HCl Material A comprises at least three of the following peaks: 11.0, 11.3, 13.5, 17.3, and 19.7° 2θ±0.2° 2θ. In one embodiment, the diffractogram of Compound I HCl Material A comprises each of the following peaks: 11.0, 11.3, 13.5, 17.3, and 19.7° 2θ±0.2° 2θ.

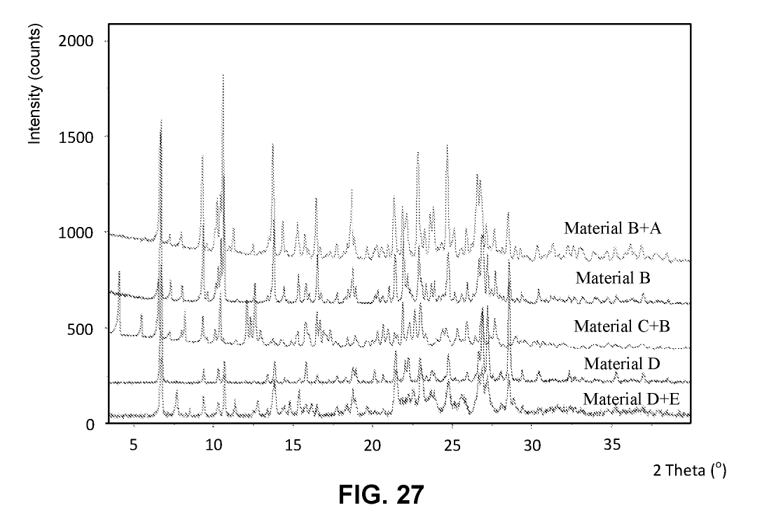

In one embodiment, Compound I HCl Material A is present as a mixture with Compound I HCl Material B (described below). In one embodiment, Compound I HCl Material A is characterized by the X-ray powder diffractogram as substantially shown in FIG. 27, which includes the presence of Compound I HCl B. FIG. 27 also includes the X-ray powder diffractograms of Compound I HCl Material B; Compound I HCl Material C, present as a mixture with Compound I HCl Material B; Compound I HCl Material D, and Compound I HCl Material E, present as a mixture with Compound I HCl Material D for comparison.

The present disclosure also provides at least one process for making Compound I HCl Material A. In one embodiment, the process comprises contacting Compound I with acetonitrile and HCl (about 3 eq.), whereby Compound I HCl Material A is formed as a mixture with Compound I HCl Material B. In one embodiment, the process for making Compound I HCl Material A is as described in the Examples provided herein.

Compound I HCl Material B

The present disclosure provides, in one embodiment, a hydrochloride complex of (2-cyclopropyl-6-(3,5-dimethylisoxazol-4-yl)-1H-benzo[d]imidazol-4-yl)di(pyridin-2-yl)methanol having a crystalline form (Compound I HCl Material B). In one embodiment, Compound I HCl Material B corresponds to an HCl salt of Compound I. In one embodiment, Compound I HCl Material B corresponds to an HCl co-crystal of Compound I.

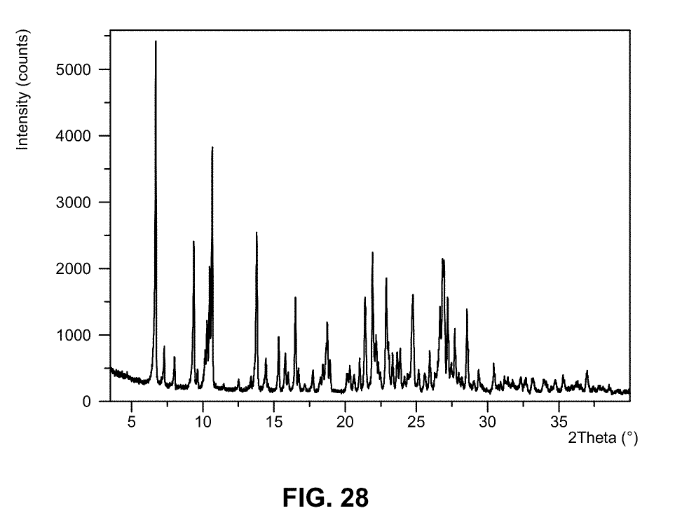

Compound I HCl Material B is characterized by an X-ray powder diffractogram comprising the following peaks: 6.7, 9.4, and 10.7° 2θ±0.2° 2θ, as determined on a diffractometer using Cu-Kα radiation at a wavelength of 1.5406 Å. In one embodiment, the diffractogram of Compound I HCl Material B further comprises one or more peaks at: 13.8, 16.5, 18.7, 21.4, 21.9, 22.9, 24.8, and 27.0° 2θ±0.2° 2θ. In one embodiment, the diffractogram of Compound I HCl Material B comprises at least two of the following peaks: 6.7, 9.4, 10.5, 10.7, 13.8, 15.3, 16.5, 18.7, 21.4, 21.9, 22.9, 24.8, 26.8, 27.0, 27.2, 27.7, and 28.6° 2θ±0.2° 2θ. In one embodiment, the diffractogram of Compound I HCl Material B comprises at least four of the following peaks: 6.7, 9.4, 10.5, 10.7, 13.8, 15.3, 16.5, 18.7, 21.4, 21.9, 22.9, 24.8, 26.8, 27.0, 27.2, 27.7, and 28.6° 2θ±0.2° 2θ. In one embodiment, the diffractogram of Compound I HCl Material B comprises at least six of the following peaks: 6.7, 9.4, 10.5, 10.7, 13.8, 15.3, 16.5, 18.7, 21.4, 21.9, 22.9, 24.8, 26.8, 27.0, 27.2, 27.7, and 28.6° 2θ±0.2° 2θ. In one embodiment, the diffractogram of Compound I HCl Material B comprises at least eight of the following peaks: 6.7, 9.4, 10.5, 10.7, 13.8, 15.3, 16.5, 18.7, 21.4, 21.9, 22.9, 24.8, 26.8, 27.0, 27.2, 27.7, and 28.6° 2θ±0.2° 2θ. In one embodiment, the diffractogram of Compound I HCl Material B comprises each of the following peaks: 6.7, 9.4, 10.5, 10.7, 13.8, 15.3, 16.5, 18.7, 21.4, 21.9, 22.9, 24.8, 26.8, 27.0, 27.2, 27.7, and 28.6° 2θ±0.2° 2θ. In one embodiment, Compound I HCl Material B is characterized by the X-ray powder diffractogram as substantially shown in FIG. 28.

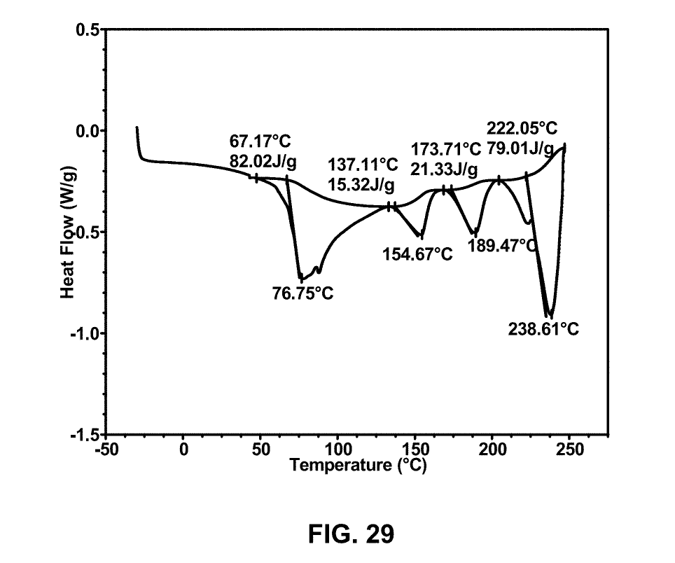

In one embodiment, Compound I HCl Material B is characterized by a differential scanning calorimeter (DSC) curve that comprises an endotherm with onset at about 222° C. In one embodiment, the DSC curve of Compound I HCl Material B shows additional endotherms with onsets at about 67° C., about 137° C., and about 174° C. In one embodiment, Compound I HCl Material B is characterized by the DSC curve as substantially shown in FIG. 29.

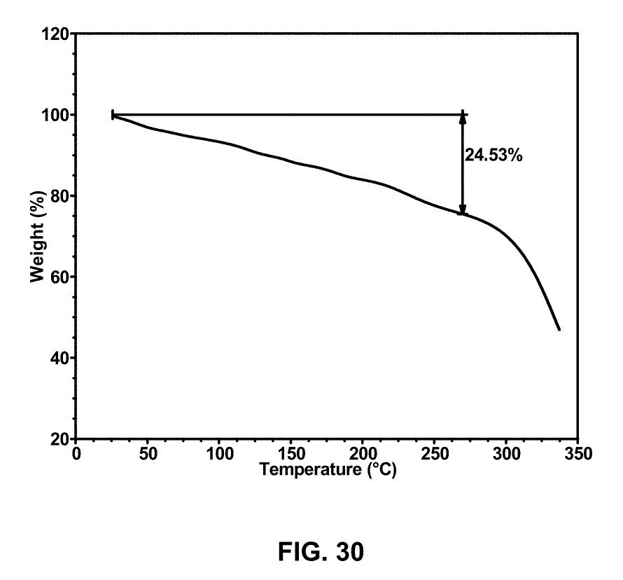

In one embodiment, Compound I HCl Material B is characterized by a thermogravimetric analysis (TGA) thermogram showing a weight loss of about 25% up to about 260° C. In one embodiment, Compound I HCl Material B is characterized by the TGA thermogram as substantially shown in FIG. 30.

In one embodiment, Compound I HCl Material B exhibits a kinetic aqueous solubility of about 6 mg/mL.

The present disclosure also provides at least one process for making Compound I HCl Material B. In one embodiment, the process comprises contacting Compound I with diethyl ether and HCl (about 3 eq.), whereby Compound I HCl Material B is formed. In one embodiment, the process for making Compound I HCl Material B is as described in the Examples provided herein.

Compound I HCl Material C

The present disclosure provides, in one embodiment, a hydrochloride complex of (2-cyclopropyl-6-(3,5-dimethylisoxazol-4-yl)-1H-benzo[d]imidazol-4-yl)di(pyridin-2-yl)methanol having a crystalline form (Compound I HCl Material C). In one embodiment, Compound I HCl Material C corresponds to an HCl salt of Compound I. In one embodiment, Compound I HCl Material C corresponds to an HCl co-crystal of Compound I.

Compound I HCl Material C is characterized by an X-ray powder diffractogram comprising the following peaks: 4.1, 8.2, and 12.6° 2θ±0.2° 2θ, as determined on a diffractometer using Cu-Kα radiation at a wavelength of 1.5406 Å. In one embodiment, the diffractogram of Compound I HCl Material C further comprises one or more peaks at: 5.4, 12.1, 12.3, 17.3, 22.6, and 25.4° 2θ±0.2° 2θ. In one embodiment, the diffractogram of Compound I HCl Material C comprises at least two of the following peaks: 4.1, 5.4, 8.2, 12.1, 12.3, 12.6, 17.3, 22.6, and 25.4° 2θ±0.2° 2θ. In one embodiment, the diffractogram of Compound I HCl Material C comprises at least four of the following peaks: 4.1, 5.4, 8.2, 12.1, 12.3, 12.6, 17.3, 22.6, and 25.4° 2θ±0.2° 2θ. In one embodiment, the diffractogram of Compound I HCl Material C comprises at least six of the following peaks: 4.1, 5.4, 8.2, 12.1, 12.3, 12.6, 17.3, 22.6, and 25.4° 2θ±0.2° 2θ. In one embodiment, the diffractogram of Compound I HCl Material C comprises each of the following peaks: 4.1, 5.4, 8.2, 12.1, 12.3, 12.6, 17.3, 22.6, and 25.4° 2θ±0.2° 2θ.

In one embodiment, Compound I HCl Material C is present as a mixture with Compound I HCl Material B. In one embodiment, Compound I HCl Material C is characterized by the X-ray powder diffractogram as substantially shown in FIG. 27, which includes the presence of Compound I HCl Material B.

The present disclosure also provides at least one process for making Compound I HCl Material C. In one embodiment, the process comprises contacting Compound I with IPA and HCl (3 eq.), whereby Compound I HCl Material A is formed as a mixture with Compound I HCl Material B. In one embodiment, the process for making Compound I HCl Material C is as described in the Examples provided herein.

Compound I HCl Material D

The present disclosure provides, in one embodiment, a hydrochloride complex of (2-cyclopropyl-6-(3,5-dimethylisoxazol-4-yl)-1H-benzo[d]imidazol-4-yl)di(pyridin-2-yl)methanol having a crystalline form (Compound I HCl Material D). In one embodiment, Compound I HCl Material D corresponds to an HCl salt of Compound I. In one embodiment, Compound I HCl Material D corresponds to an HCl co-crystal of Compound I.

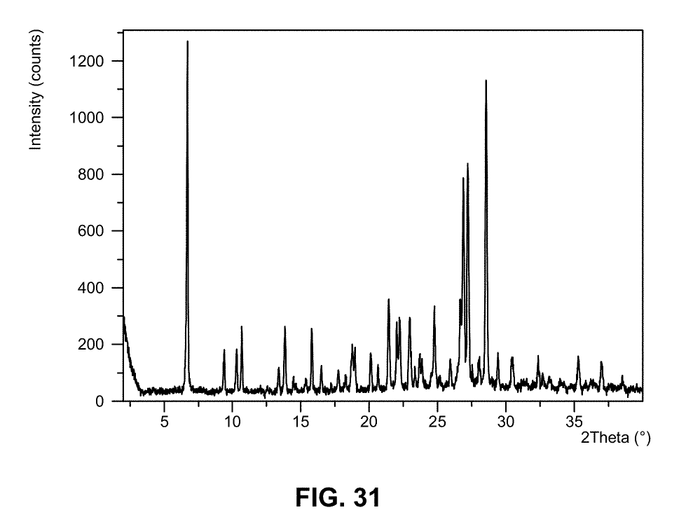

Compound I HCl Material D is characterized by an X-ray powder diffractogram comprising the following peaks: 6.7, 27.2, and 28.5° 2θ±0.2° 2θ, as determined on a diffractometer using Cu-Kα radiation at a wavelength of 1.5406 Å. In one embodiment, the diffractogram of Compound I HCl Material D further comprises one or more peaks at: 10.7, 13.9, 15.8, 21.4, 22.2, 23.0, 24.7, and 26.9° 2θ±0.2° 2θ. In one embodiment, the diffractogram of Compound I HCl Material D comprises at least two of the following peaks: 6.7, 9.4, 10.3, 10.7, 13.9, 15.8, 18.7, 19.0, 20.1, 21.4, 22.0, 22.2, 23.0, 24.7, 26.6, 26.9, 27.2, and 28.5° 2θ±0.2° 2θ. In one embodiment, the diffractogram of Compound I HCl Material D comprises at least four of the following peaks: 6.7, 9.4, 10.3, 10.7, 13.9, 15.8, 18.7, 19.0, 20.1, 21.4, 22.0, 22.2, 23.0, 24.7, 26.6, 26.9, 27.2, and 28.5° 2θ±0.2° 2θ. In one embodiment, the diffractogram of Compound I HCl Material D comprises at least six of the following peaks: 6.7, 9.4, 10.3, 10.7, 13.9, 15.8, 18.7, 19.0, 20.1, 21.4, 22.0, 22.2, 23.0, 24.7, 26.6, 26.9, 27.2, and 28.5° 2θ±0.2° 2θ. In one embodiment, the diffractogram of Compound I HCl Material D comprises at least eight of the following peaks: 6.7, 9.4, 10.3, 10.7, 13.9, 15.8, 18.7, 19.0, 20.1, 21.4, 22.0, 22.2, 23.0, 24.7, 26.6, 26.9, 27.2, and 28.5° 2θ±0.2° 2θ. In one embodiment, the diffractogram of Compound I HCl Material D comprises each of the following peaks: 6.7, 9.4, 10.3, 10.7, 13.9, 15.8, 18.7, 19.0, 20.1, 21.4, 22.0, 22.2, 23.0, 24.7, 26.6, 26.9, 27.2, and 28.5° 2θ±0.2° 2θ. In one embodiment, Compound I HCl Material D is characterized by the X-ray powder diffractogram as substantially shown in FIG. 31.

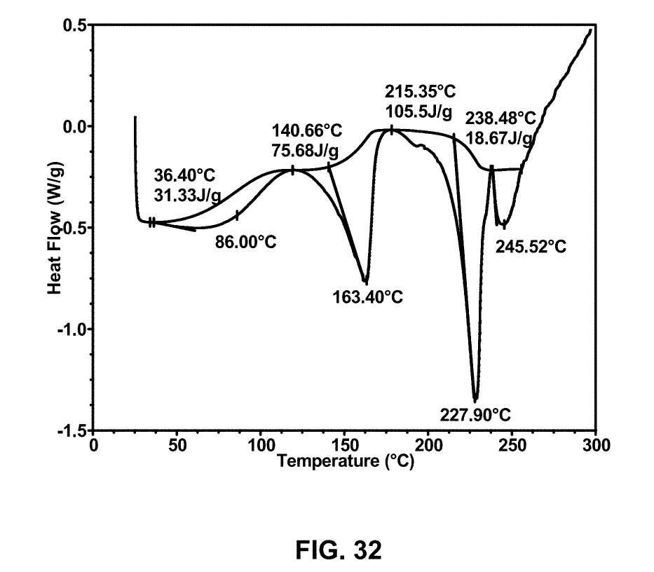

In one embodiment, Compound I HCl Material D is characterized by a differential scanning calorimeter (DSC) curve that comprises an endotherm with onset at about 238° C. In one embodiment, the DSC curve of Compound I HCl Material D shows additional endotherms with onsets at about 36° C., about 141° C., about 215° C., and about 246° C. In one embodiment, Compound I HCl Material D is characterized by the DSC curve as substantially shown in FIG. 32.

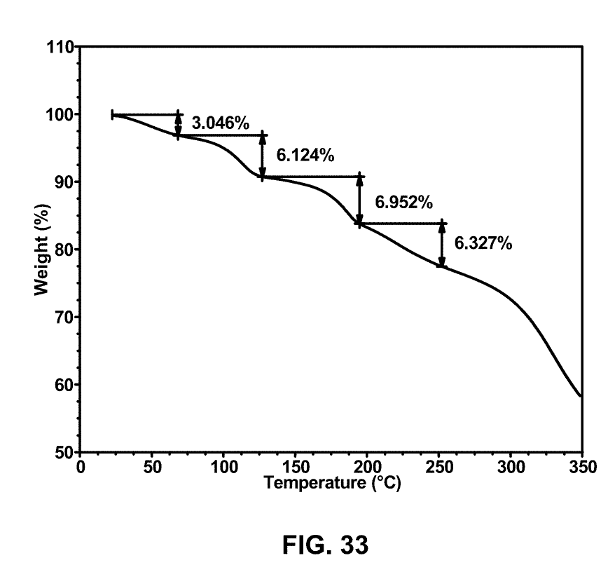

In one embodiment, Compound I HCl Material D is characterized by a thermogravimetric analysis (TGA) thermogram showing multiple weight losses up to about 260° C. In one embodiment, Compound I HCl Material D is characterized by a thermogravimetric analysis (TGA) thermogram showing a total weight loss of about 22%. Compound I HCl Material D is characterized by the TGA thermogram as substantially shown in FIG. 33.

The present disclosure also provides at least one process for making Compound I HCl Material C. In one embodiment, the process comprises contacting Compound I with IPA, 1-propanol, MEK, or 2-MeTHF in the presence of HCl, whereby Compound I HCl Material C is formed. In one embodiment, the process for making Compound I HCl Material D is as described in the Examples provided herein.

Compound I HCl Material E

The present disclosure provides, in one embodiment, a hydrochloride complex of (2-cyclopropyl-6-(3,5-dimethylisoxazol-4-yl)-1H-benzo[d]imidazol-4-yl)di(pyridin-2-yl)methanol having a crystalline form (Compound I HCl Material E). In one embodiment, Compound I HCl Material E corresponds to an HCl salt of Compound I. In one embodiment, Compound I HCl Material E corresponds to an HCl co-crystal of Compound I.

Compound I HCl Material E is characterized by an X-ray powder diffractogram comprising the following peaks: 7.7, 12.8, and 15.4° 2θ±0.2° 2θ, as determined on a diffractometer using Cu-Kα radiation at a wavelength of 1.5406 Å. In one embodiment, the diffractogram of Compound I HCl Material E further comprises one or more peaks at: 11.3, 14.8, 16.2, 22.5, and 28.9° 2θ±0.2° 2θ. In one embodiment, the diffractogram of Compound I HCl Material E comprises at least two of the following peaks: 7.7, 11.3, 12.8, 14.8, 15.4, 16.2, 22.5, and 28.9° 2θ±0.2° 2θ. In one embodiment, the diffractogram of Compound I HCl Material E comprises at least four of the following peaks: 7.7, 11.3, 12.8, 14.8, 15.4, 16.2, 22.5, and 28.9° 2θ±0.2° 2θ. In one embodiment, the diffractogram of Compound I HCl Material E comprises at least six of the following peaks: 7.7, 11.3, 12.8, 14.8, 15.4, 16.2, 22.5, and 28.9° 2θ±0.2° 2θ. In one embodiment, the diffractogram of Compound I HCl Material E comprises each of the following peaks: 7.7, 11.3, 12.8, 14.8, 15.4, 16.2, 22.5, and 28.9° 2θ±0.2° 2θ.

In one embodiment, Compound I HCl Material E is present as a mixture with Compound I HCl Material D. In one embodiment, Compound I HCl Material E is characterized by the X-ray powder diffractogram as substantially shown in FIG. 27, which includes the presence of Compound I HCl Material D.

In one embodiment, Compound I HCl Material E is characterized as a DCM solvate.

The present disclosure also provides at least one process for making Compound I HCl Material E. In one embodiment, the process comprises contacting Compound I with DCM, a mixture of DCM and IPA, or a mixture of DCM and EtOH in the presence of HCl, whereby Compound I HCl Material E is formed as mixture with Compound I HCl Material D. In one embodiment, the process for making Compound I HCl Material E is as described in the Examples provided herein.

Compound I Sulfate Material A

The present disclosure provides, in one embodiment, a sulfate complex of (2-cyclopropyl-6-(3,5-dimethylisoxazol-4-yl)-1H-benzo[d]imidazol-4-yl)di(pyridin-2-yl)methanol having a crystalline form (Compound I sulfate Material A). In one embodiment, Compound I sulfate Material A corresponds to a sulfate salt of Compound I. In one embodiment, Compound I sulfate Material A corresponds to a sulfate co-crystal of Compound I.

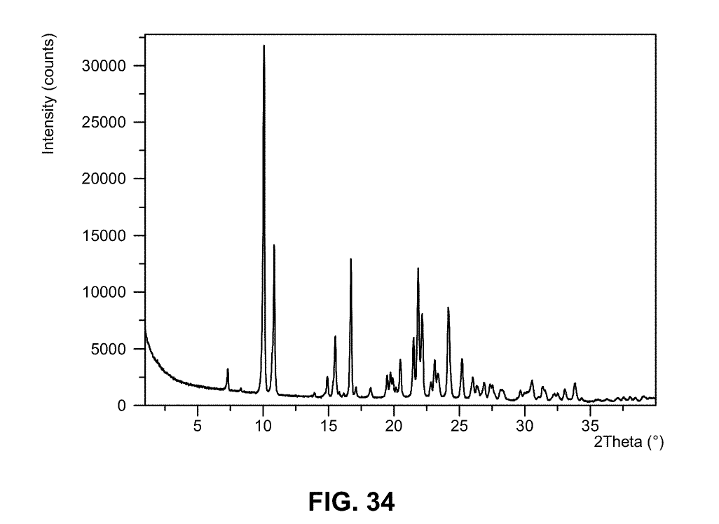

Compound I sulfate Material A is characterized by an X-ray powder diffractogram comprising the following peaks: 10.1, 10.9, and 16.7° 2θ±0.2° 2θ, as determined on a diffractometer using Cu-Kα radiation at a wavelength of 1.5406 Å. In one embodiment, the diffractogram of Compound I sulfate Material A further comprises one or more peaks at: 7.3, 15.5, 21.5, 21.9, 22.2, 24.1, and 25.2° 2θ±0.2° 2θ. In one embodiment, the diffractogram of Compound I sulfate Material A comprises at least two, or at least four, or at least six, or at least eight, or all of the following peaks: 7.3, 10.1, 10.7, 10.9, 14.9, 15.5, 16.7, 19.5, 19.7, 19.9, 20.5, 21.5, 21.9, 22.2, 23.1, 23.4, 24.1, 25.2, 26.0, and 30.6° 2θ±0.2° 2θ. In one embodiment, the diffractogram of Compound I sulfate Material A comprises at least four of the following peaks: 7.3, 10.1, 10.7, 10.9, 14.9, 15.5, 16.7, 19.5, 19.7, 19.9, 20.5, 21.5, 21.9, 22.2, 23.1, 23.4, 24.1, 25.2, 26.0, and 30.6° 2θ±0.2° 2θ. In one embodiment, the diffractogram of Compound I sulfate Material A comprises at least six of the following peaks: 7.3, 10.1, 10.7, 10.9, 14.9, 15.5, 16.7, 19.5, 19.7, 19.9, 20.5, 21.5, 21.9, 22.2, 23.1, 23.4, 24.1, 25.2, 26.0, and 30.6° 2θ±0.2° 2θ. In one embodiment, the diffractogram of Compound I sulfate Material A comprises at least eight of the following peaks: 7.3, 10.1, 10.7, 10.9, 14.9, 15.5, 16.7, 19.5, 19.7, 19.9, 20.5, 21.5, 21.9, 22.2, 23.1, 23.4, 24.1, 25.2, 26.0, and 30.6° 2θ±0.2° 2θ. In one embodiment, the diffractogram of Compound I sulfate Material A comprises each of the following peaks: 7.3, 10.1, 10.7, 10.9, 14.9, 15.5, 16.7, 19.5, 19.7, 19.9, 20.5, 21.5, 21.9, 22.2, 23.1, 23.4, 24.1, 25.2, 26.0, and 30.6° 2θ±0.2° 2θ. In one embodiment, Compound I sulfate Material A is characterized by the X-ray powder diffractogram as substantially shown in FIG. 34.

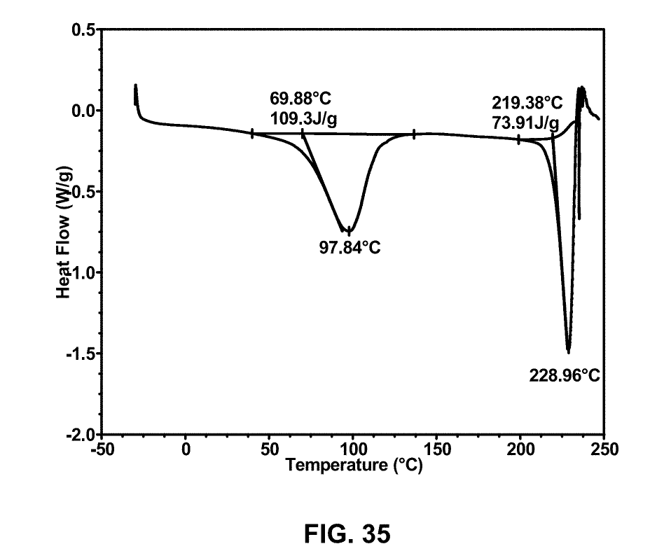

In one embodiment, Compound I sulfate Material A is characterized by a differential scanning calorimeter (DSC) curve that comprises an endotherm at about 219° C. In one embodiment, the DSC curve of Compound I sulfate Material A shows an additional endotherm with onset at about 70° C. In one embodiment, Compound I sulfate Material A is characterized by the DSC curve as substantially shown in FIG. 35.

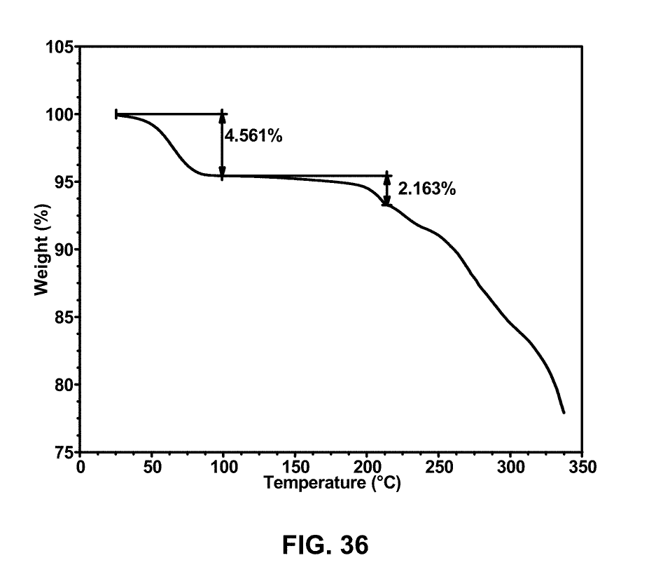

In one embodiment, Compound I sulfate Material A is characterized by a thermogravimetric analysis (TGA) thermogram showing a weight loss of about 4.6% from about 23° C. to about 92° C. In one embodiment, the TGA thermogram of Compound I sulfate Material A additionally shows a weight loss of about 2% weight loss between about 100° C. and about 220° C. In one embodiment, Compound I sulfate Material A is characterized by the TGA thermogram as substantially shown in FIG. 36.

In one embodiment, Compound I sulfate Material A is characterized as a hydrate. In one embodiment, a mixture of Compound I sulfate Material A and Compound I sulfate Material B exhibits a kinetic aqueous solubility of about 4 mg/mL.

The present disclosure also provides at least a process for making a mixture eof Compound I sulfate Material A and Compound I sulfate Material B. In one embodiment, the process comprises volume reduction of a solution comprising Compound I, IPA, MeOH, and sulfuric acid or vacuum drying of solids isolated from a slurry comprising Compound I, IPA, and sulfuric acid. It is of note that embodiments involving the vacuum drying of the aforementioned solids from the slurry may result in an X-ray powder diffractogram having one or more peaks shifted relative to those described above and in FIG. 34. In one embodiment, the process of making Compound I sulfate Material A comprises vacuum drying Compound I sulfate Material B. In one embodiment, the process for making Compound I sulfate Material A is as described in the Examples provided herein.

Compound I Sulfate Material B

The present disclosure provides, in one embodiment, a sulfate complex of (2-cyclopropyl-6-(3,5-dimethylisoxazol-4-yl)-1H-benzo[d]imidazol-4-yl)di(pyridin-2-yl)methanol having a crystalline form (Compound I sulfate Material B). In one embodiment, Compound I sulfate Material B corresponds to a sulfate salt of Compound I. In one embodiment, Compound I sulfate Material B corresponds to a sulfate co-crystal of Compound I.

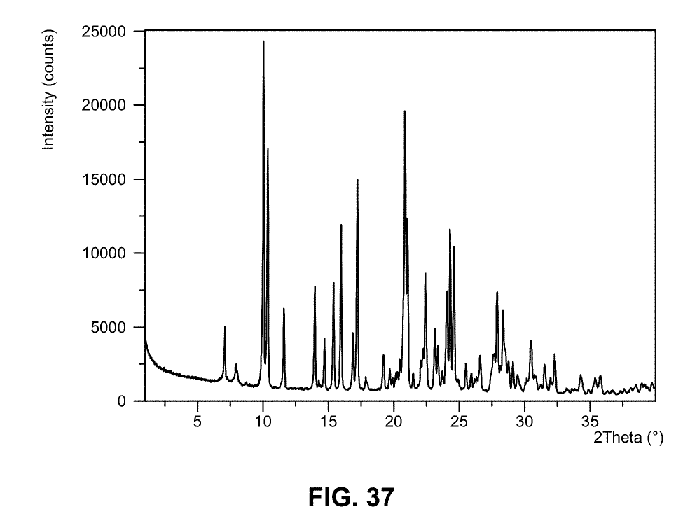

Compound I sulfate Material B is characterized by an X-ray powder diffractogram comprising the following peaks: 10.1, 17.2, and 20.9° 2θ±0.2° 2θ, as determined on a diffractometer using Cu-Kα radiation at a wavelength of 1.5406 Å. In one embodiment, the diffractogram of Compound I sulfate Material B further comprises one or more peaks at: 7.1, 10.4, 11.6, 14.0, 15.4, 16.0, 21.1, 22.4, 24.1, 24.3, 24.6, and 27.9° 2θ±0.2° 2θ. In one embodiment, the diffractogram of Compound I sulfate Material B comprises at least two of the following peaks: 7.1, 10.1, 10.4, 11.6, 14.0, 14.7, 15.4, 16.0, 16.9, 17.2, 19.2, 20.9, 21.1, 22.4, 23.1, 24.1, 24.3, 24.6, 27.9, 28.3, 30.5, and 32.3° 2θ±0.2° 2θ. In one embodiment, the diffractogram of Compound I sulfate Material B comprises at least four of the following peaks: 7.1, 10.1, 10.4, 11.6, 14.0, 14.7, 15.4, 16.0, 16.9, 17.2, 19.2, 20.9, 21.1, 22.4, 23.1, 24.1, 24.3, 24.6, 27.9, 28.3, 30.5, and 32.3° 2θ±0.2° 2θ. In one embodiment, the diffractogram of Compound I sulfate Material B comprises at least six of the following peaks: 7.1, 10.1, 10.4, 11.6, 14.0, 14.7, 15.4, 16.0, 16.9, 17.2, 19.2, 20.9, 21.1, 22.4, 23.1, 24.1, 24.3, 24.6, 27.9, 28.3, 30.5, and 32.3° 2θ±0.2° 2θ. In one embodiment, the diffractogram of Compound I sulfate Material B comprises at least eight of the following peaks: 7.1, 10.1, 10.4, 11.6, 14.0, 14.7, 15.4, 16.0, 16.9, 17.2, 19.2, 20.9, 21.1, 22.4, 23.1, 24.1, 24.3, 24.6, 27.9, 28.3, 30.5, and 32.3° 2θ±0.2° 2θ. In one embodiment, the diffractogram of Compound I sulfate Material B comprises each of the following peaks: 7.1, 10.1, 10.4, 11.6, 14.0, 14.7, 15.4, 16.0, 16.9, 17.2, 19.2, 20.9, 21.1, 22.4, 23.1, 24.1, 24.3, 24.6, 27.9, 28.3, 30.5, and 32.3° 2θ±0.2° 2θ. In one embodiment, Compound I sulfate Material B is characterized by the X-ray powder diffractogram as substantially shown in FIG. 37.

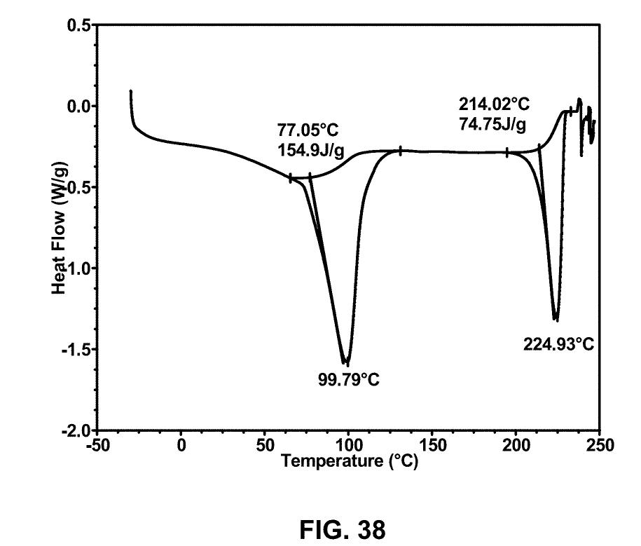

In one embodiment, Compound I sulfate Material B is characterized by a differential scanning calorimeter (DSC) curve that comprises an endotherm with onset at about 214° C. In one embodiment, the DSC curve of Compound I sulfate Material B shows an additional endotherm with onset at about 77° C. In one embodiment, Compound I sulfate Material B is characterized by the DSC curve as substantially shown in FIG. 38.

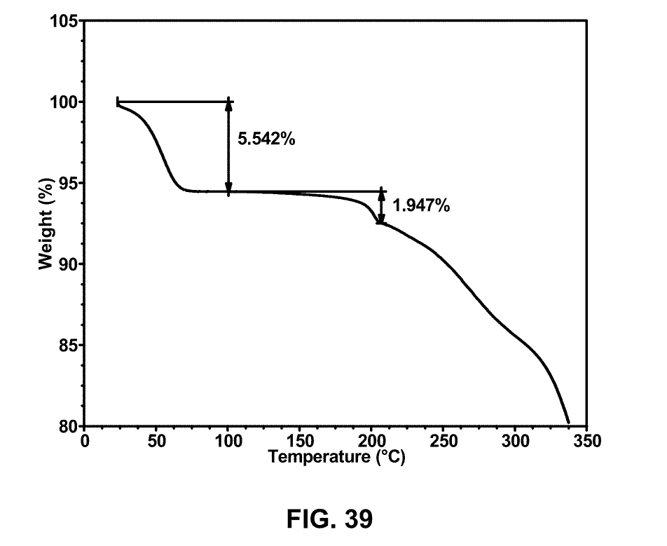

In one embodiment, Compound I sulfate Material B is characterized by a thermogravimetric analysis (TGA) thermogram showing a weight loss of about 5.5% from about 23° C. to about 92° C. In one embodiment, the TGA thermogram of Compound I sulfate Material B additionally shows a weight loss of about 2% between about 100° C. and about 200° C. In one embodiment, Compound I sulfate Material B is characterized by the TGA thermogram as substantially shown in FIG. 39.

In one embodiment, Compound I sulfate Material B exhibits a kinetic aqueous solubility of about 4 mg/mL.

The present disclosure also provides at least one process for making Compound I sulfate Material B. In one embodiment, the process comprises contacting Compound I with IPA and sulfuric acid (e.g., about 1 equivalent of sulfuric acid), whereby Compound I sulfate Material B is formed. In one embodiment, the process for making Compound I sulfate Material B is as described in the Examples provided herein.

Compound I Sulfate Material C

The present disclosure provides, in one embodiment, a sulfate complex of (2-cyclopropyl-6-(3,5-dimethylisoxazol-4-yl)-1H-benzo[d]imidazol-4-yl)di(pyridin-2-yl)methanol having a crystalline form (Compound I sulfate Material C). In one embodiment, Compound I sulfate Material C corresponds to a sulfate salt of Compound I. In one embodiment, Compound I sulfate Material C corresponds to a sulfate co-crystal of Compound I.

Compound I sulfate Material C is characterized by an X-ray powder diffractogram comprising the following peaks: 10.7, 16.1, and 18.6° 2θ±0.2° 2θ, as determined on a diffractometer using Cu-Kα radiation at a wavelength of 1.5406 Å. In one embodiment, the diffractogram of Compound I sulfate Material C further comprises one or more peaks at: 13.4, 17.2, and 20.3° 2θ±0.2° 2θ. In one embodiment, the diffractogram of Compound I sulfate Material C comprises at least two of the following peaks: 10.7, 13.4, 16.1, 17.2, 18.6, and 20.3° 2θ±0.2° 2θ. In one embodiment, the diffractogram of Compound I sulfate Material C comprises at least four of the following peaks: 10.7, 13.4, 16.1, 17.2, 18.6, and 20.3° 2θ±0.2° 2θ. In one embodiment, the diffractogram of Compound I sulfate Material C comprises each of the following peaks: 10.7, 13.4, 16.1, 17.2, 18.6, and 20.3° 2θ±0.2° 2θ.

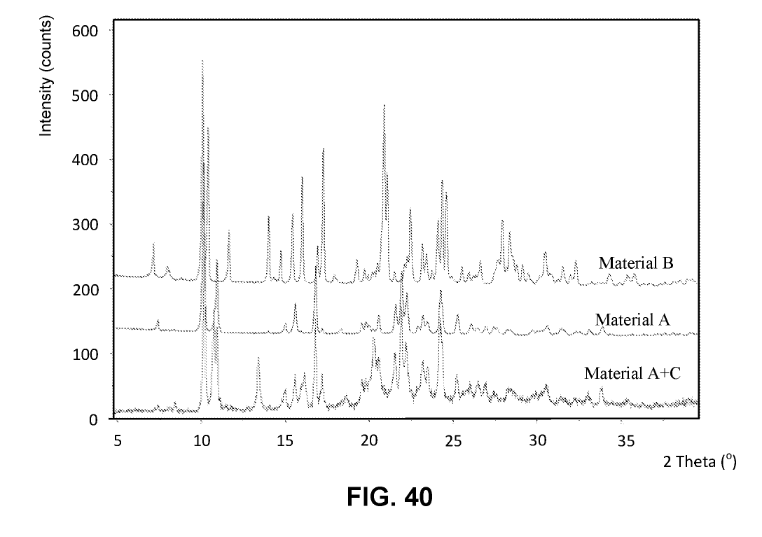

In one embodiment, Compound I sulfate Material C is present as a mixture with Compound I sulfate Material A. In one embodiment, Compound I sulfate Material C is characterized by the X-ray powder diffractogram as substantially shown in FIG. 40, which includes the presence of Compound I sulfate Material A. FIG. 40 also includes the X-ray powder diffractograms of Compound I sulfate Material A, and Compound I sulfate Material B for comparison.

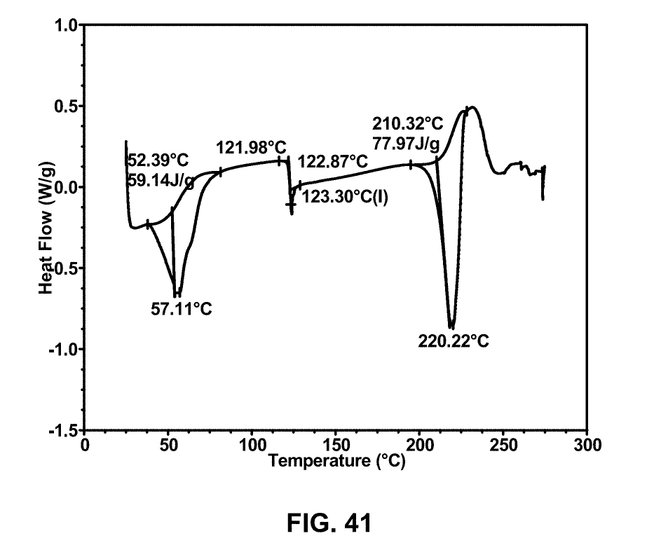

In one embodiment, Compound I sulfate Material C, present as a mixture with Compound I sulfate Material A, is characterized by a differential scanning calorimeter (DSC) curve that comprises an endotherm with onset at about 210° C. In one embodiment, the DSC curve of Compound I sulfate Material C, as a mixture with Compound I sulfate Material A, comprises an additional endotherm with onset at about 52° C. In one embodiment, Compound I sulfate Material C, as a mixture with Compound I sulfate Material A, is characterized by the DSC curve as substantially shown in FIG. 41.

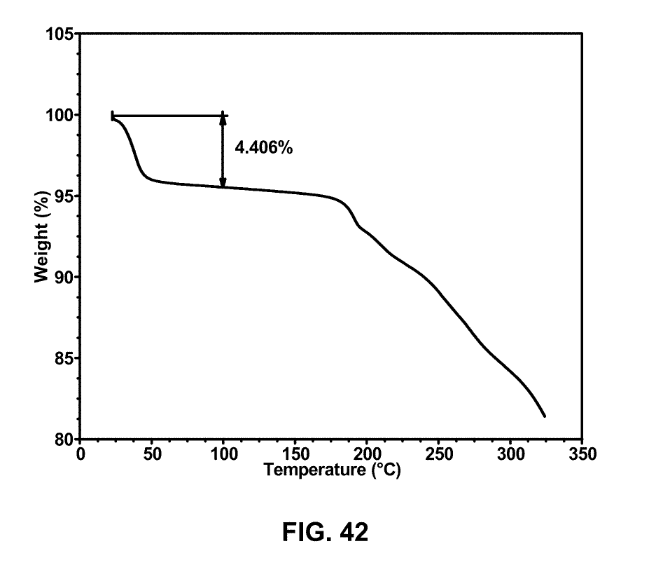

In one embodiment, Compound I sulfate Material C, present as a mixture with Compound I sulfate Material A, is characterized by a thermogravimetric analysis (TGA) thermogram showing a weight loss of about 4.4% below about 100° C. In one embodiment, Compound I sulfate Material C, as a mixture with Compound I sulfate Material A, is characterized by the TGA thermogram as substantially shown in FIG. 42.

In one embodiment Compound I sulfate Material C, present as a mixture with Compound I sulfate Material A, comprises about 0.68% water as measured by KF analysis. In one embodiment, Compound I sulfate Material C is characterized as an isopropanol (IPA) solvate.

The present disclosure also provides at least one process for making Compound I sulfate Material C. In one embodiment, the process comprises contacting Compound I with IPA and sulfuric acid, whereby Compound I sulfate Material C is formed as mixture with Compound I sulfate Material A. In one embodiment, the process for making Compound I sulfate Material C is as described in the Examples provided herein.

Compound I Tosylate Form I

The present disclosure provides, in one embodiment, a tosylate complex of (2-cyclopropyl-6-(3,5-dimethylisoxazol-4-yl)-1H-benzo[d]imidazol-4-yl)di(pyridin-2-yl)methanol having a crystalline form (Compound I tosylate Form I). In one embodiment, Compound I tosylate Form I corresponds to a tosylate salt of Compound I. In one embodiment, Compound I tosylate Form I corresponds to a tosylate co-crystal of Compound I.

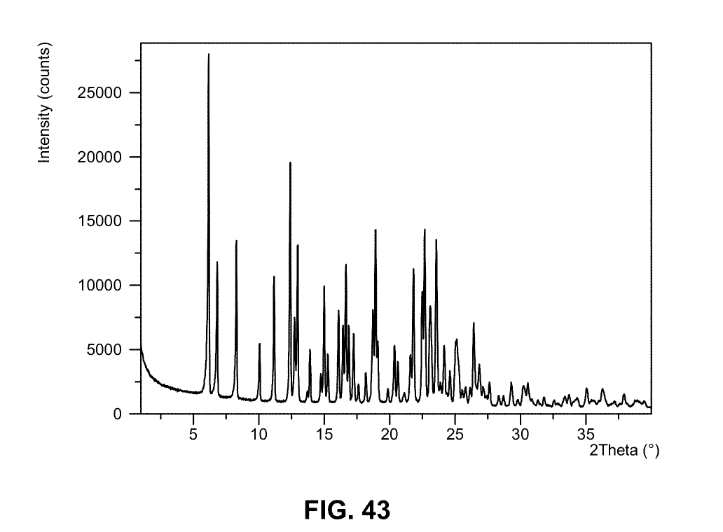

Compound I tosylate Form I is characterized by an X-ray powder diffractogram comprising the following peaks: 6.2, 11.2, and 13.0° 2θ±0.2° 2θ, as determined on a diffractometer using Cu-Kα radiation at a wavelength of 1.5406 Å. In one embodiment, the diffractogram of Compound I tosylate Form I further comprises one or more peaks at: 6.8, 11.2, 12.4, 15.0, 16.7, 18.9, 21.8, 22.7, 23.6, and 26.4° 2θ±0.2° 2θ. In one embodiment, the diffractogram of Compound I tosylate Form I comprises at least two of the following peaks: 6.2, 6.8, 8.3, 10.1, 11.2, 12.4, 12.8, 13.0, 13.9, 15.0, 15.3, 16.1, 16.7, 17.3, 18.7, 18.9, 20.4, 21.8, 22.7, 23.1, 23.6, 25.1, and 26.4° 2θ±0.2° 2θ. In one embodiment, the diffractogram of Compound I tosylate Form I comprises at least four of the following peaks: 6.2, 6.8, 8.3, 10.1, 11.2, 12.4, 12.8, 13.0, 13.9, 15.0, 15.3, 16.1, 16.7, 17.3, 18.7, 18.9, 20.4, 21.8, 22.7, 23.1, 23.6, 25.1, and 26.4° 2θ±0.2° 2θ. In one embodiment, the diffractogram of Compound I tosylate Form I comprises at least six of the following peaks: 6.2, 6.8, 8.3, 10.1, 11.2, 12.4, 12.8, 13.0, 13.9, 15.0, 15.3, 16.1, 16.7, 17.3, 18.7, 18.9, 20.4, 21.8, 22.7, 23.1, 23.6, 25.1, and 26.4° 2θ±0.2° 2θ. In one embodiment, the diffractogram of Compound I tosylate Form I comprises at least eight of the following peaks: 6.2, 6.8, 8.3, 10.1, 11.2, 12.4, 12.8, 13.0, 13.9, 15.0, 15.3, 16.1, 16.7, 17.3, 18.7, 18.9, 20.4, 21.8, 22.7, 23.1, 23.6, 25.1, and 26.4° 2θ±0.2° 2θ. In one embodiment, the diffractogram of Compound I tosylate Form I comprises each of the following peaks: 6.2, 6.8, 8.3, 10.1, 11.2, 12.4, 12.8, 13.0, 13.9, 15.0, 15.3, 16.1, 16.7, 17.3, 18.7, 18.9, 20.4, 21.8, 22.7, 23.1, 23.6, 25.1, and 26.4° 2θ±0.2° 2θ. In one embodiment, Compound I tosylate Form I is characterized by the X-ray powder diffractogram as substantially shown in FIG. 43.

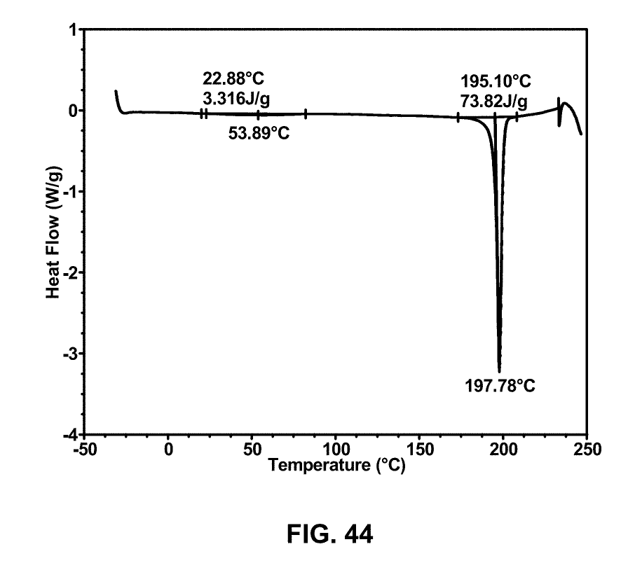

In one embodiment, Compound I tosylate Form I is characterized by a differential scanning calorimeter (DSC) curve that comprises an endotherm with onset at about 195° C. In one embodiment, the DSC curve of Compound I tosylate Form I shows an additional endotherm with onset at about 23° C. In one embodiment, Compound I tosylate Form I is characterized by the DSC curve as substantially shown in FIG. 44.

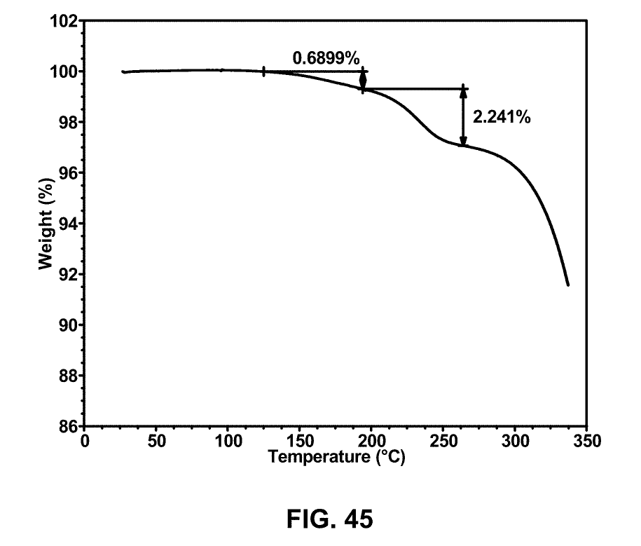

In one embodiment, Compound I tosylate Form I is characterized by a thermogravimetric analysis (TGA) thermogram showing no weight loss prior to about 130° C. In one embodiment, the TGA thermogram of Compound I tosylate Form I additionally shows a series of weight loss steps above 130° C. (e.g., a weight loss of about 0.7% from about 130° C. to about 200° C., a weight loss of about 2% from 200° C. to about 260° C., etc.). In one embodiment, Compound I tosylate Form I is characterized by the TGA thermogram as substantially shown in FIG. 45.

In one embodiment, Compound I tosylate Form I exhibits a kinetic aqueous solubility of about 3 mg/mL.

The present disclosure also provides at least one process for making Compound I tosylate Form I. In one embodiment, the process comprises contacting Compound I with MEK and p-toluenesulfonic acid (about 1 eq.), whereby Compound I tosylate Form I is formed. In one embodiment, the process for making Compound I tosylate Form I is as described in the Examples provided herein.

Compound I Tosylate Material A

The present disclosure provides, in one embodiment, a tosylate complex of (2-cyclopropyl-6-(3,5-dimethylisoxazol-4-yl)-1H-benzo[d]imidazol-4-yl)di(pyridin-2-yl)methanol having a crystalline form (Compound I tosylate Material A). In one embodiment, Compound I tosylate Material A corresponds to a tosylate salt of Compound I. In one embodiment, Compound I tosylate Material A corresponds to a tosylate co-crystal of Compound I.

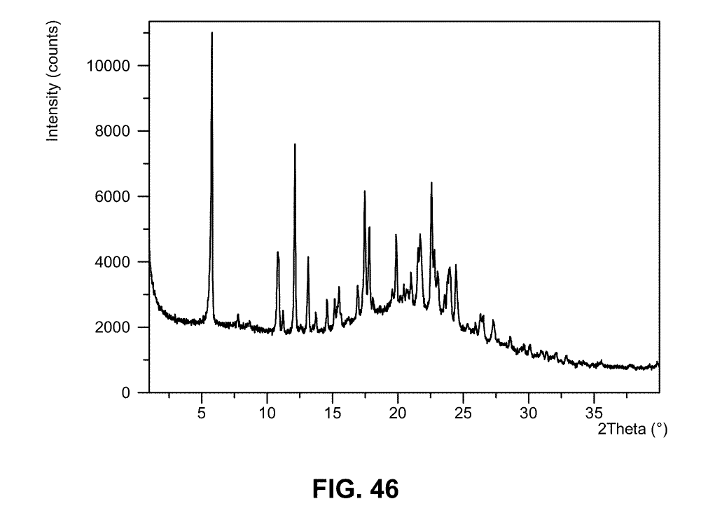

Compound I tosylate Material A is characterized by an X-ray powder diffractogram comprising the following peaks: 5.8, 12.1, and 22.6° 2θ±0.2° 2θ, as determined on a diffractometer using Cu-Kα radiation at a wavelength of 1.5406 Å. In one embodiment, the diffractogram of Compound I tosylate Material A further comprises one or more peaks at: 10.8, 13.2, 17.5, 17.8, 19.9, 21.7, and 24.4° 2θ±0.2° 2θ. In one embodiment, the diffractogram of Compound I tosylate Material A comprises at least two, or at least four, or at least six, or at least eight, or all of the following peaks: 5.8, 10.8, 12.1, 13.2, 14.6, 15.2, 15.5, 16.9, 17.5, 17.8, 19.9, 21.7, 22.6, 22.8, 23.1, 23.8, 24.0, and 24.4° 2θ±0.2° 2θ. In one embodiment, the diffractogram of Compound I tosylate Material A comprises at least four of the following peaks: 5.8, 10.8, 12.1, 13.2, 14.6, 15.2, 15.5, 16.9, 17.5, 17.8, 19.9, 21.7, 22.6, 22.8, 23.1, 23.8, 24.0, and 24.4° 2θ±0.2° 2θ. In one embodiment, the diffractogram of Compound I tosylate Material A comprises at least six of the following peaks: 5.8, 10.8, 12.1, 13.2, 14.6, 15.2, 15.5, 16.9, 17.5, 17.8, 19.9, 21.7, 22.6, 22.8, 23.1, 23.8, 24.0, and 24.4° 2θ±0.2° 2θ. In one embodiment, the diffractogram of Compound I tosylate Material A comprises at least eight of the following peaks: 5.8, 10.8, 12.1, 13.2, 14.6, 15.2, 15.5, 16.9, 17.5, 17.8, 19.9, 21.7, 22.6, 22.8, 23.1, 23.8, 24.0, and 24.4° 2θ±0.2° 2θ. In one embodiment, the diffractogram of Compound I tosylate Material A comprises each of the following peaks: 5.8, 10.8, 12.1, 13.2, 14.6, 15.2, 15.5, 16.9, 17.5, 17.8, 19.9, 21.7, 22.6, 22.8, 23.1, 23.8, 24.0, and 24.4° 2θ±0.2° 2θ. In one embodiment, Compound I tosylate Material A is characterized by the X-ray powder diffractogram as substantially shown in FIG. 46, which includes the presence of amorphous material.

The present disclosure also provides at least one process for making Compound I tosylate Material A. In one embodiment, the process comprises contacting Compound I with EtOAc, heptane, and p-toluenesulfonic acid (about 1 eq.), whereby Compound I tosylate Material A is formed. In one embodiment, the process for making Compound I tosylate Material A is as described in the Examples provided herein.

Compound I Tosylate Material C

The present disclosure provides, in one embodiment, a tosylate complex of (2-cyclopropyl-6-(3,5-dimethylisoxazol-4-yl)-1H-benzo[d]imidazol-4-yl)di(pyridin-2-yl)methanol having a crystalline form (Compound I tosylate Material C). In one embodiment, Compound I tosylate Material C corresponds to a tosylate salt of Compound I. In one embodiment, Compound I tosylate Material C corresponds to a tosylate co-crystal of Compound I.

Compound I tosylate Material C is characterized by an X-ray powder diffractogram comprising the following peaks: 6.0, 11.7, and 14.5° 2θ±0.2° 2θ, as determined on a diffractometer using Cu-Kα radiation at a wavelength of 1.5406 Å. In one embodiment, the diffractogram of Compound I tosylate Material C further comprises one or more peaks at 9.9, 12.0, 15.4, and 20.9° 2θ±0.2° 2θ. In one embodiment, the diffractogram of Compound I tosylate Material C comprises at least two of the following peaks: 6.0, 9.9, 11.7, 12.0, 14.5, 15.4, and 20.9° 2θ±0.2° 2θ. In one embodiment, the diffractogram of Compound I tosylate Material C comprises at least four of the following peaks: 6.0, 9.9, 11.7, 12.0, 14.5, 15.4, and 20.9° 2θ±0.2° 2θ. In one embodiment, the diffractogram of Compound I tosylate Material C comprises at least six of the following peaks: 6.0, 9.9, 11.7, 12.0, 14.5, 15.4, and 20.9° 2θ±0.2° 2θ. In one embodiment, the diffractogram of Compound I tosylate Material C comprises each of the following peaks: 6.0, 9.9, 11.7, 12.0, 14.5, 15.4, and 20.9° 2θ±0.2° 2θ.

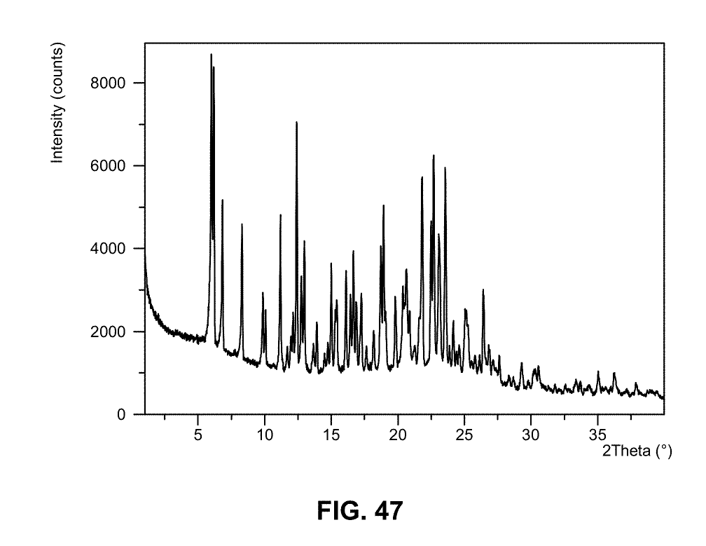

In one embodiment, Compound I tosylate Material C is present as a mixture with Compound I tosylate Form I. In one embodiment, Compound I tosylate Material C is characterized by the X-ray powder diffractogram as substantially shown in FIG. 47, which includes the presence of Compound I tosylate Form I.

The present disclosure also provides at least one process for making Compound I tosylate Material C. In one embodiment, the process comprises contacting Compound I with EtOAc and p-toluenesulfonic acid (about 2 eq.), whereby Compound I tosylate Material C is formed as a mixture with Compound I tosylate Form I. In one embodiment, the process for making Compound I tosylate Material C is as described in the Examples provided herein.

Compound I Edisylate Material A

The present disclosure provides, in one embodiment, an edisylate complex of (2-cyclopropyl-6-(3,5-dimethylisoxazol-4-yl)-1H-benzo[d]imidazol-4-yl)di(pyridin-2-yl)methanol having a crystalline form (Compound I edisylate Material A). In one embodiment, Compound I edisylate Material A corresponds to an edisylate salt of Compound I. In one embodiment, Compound I edisylate Material A corresponds to an edisylate co-crystal of Compound I.

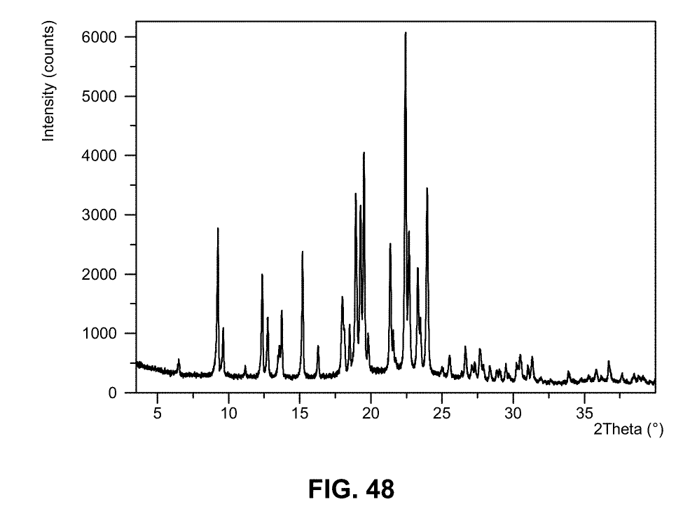

Compound I edisylate Material A is characterized by an X-ray powder diffractogram comprising the following peaks: 18.9, 19.5, and 22.4° 2θ±0.2° 2θ, as determined on a diffractometer using Cu-Kα radiation at a wavelength of 1.5406 Å. In one embodiment, the diffractogram of Compound I edisylate Material A further comprises one or more peaks at: 9.3, 12.4, 15.2, 18.0, 19.3, 21.3, and 24.0° 2θ±0.2° 2θ. In one embodiment, the diffractogram of Compound I edisylate Material A comprises at least two of the following peaks: 9.3, 9.6, 12.4, 12.8, 13.8, 15.2, 18.0, 18.9, 19.3, 19.5, 21.3, and 22.4° 2θ±0.2° 2θ. In one embodiment, the diffractogram of Compound I edisylate Material A comprises at least four of the following peaks: 9.3, 9.6, 12.4, 12.8, 13.8, 15.2, 18.0, 18.9, 19.3, 19.5, 21.3, and 22.4° 2θ±0.2° 2θ. In one embodiment, the diffractogram of Compound I edisylate Material A comprises at least six of the following peaks: 9.3, 9.6, 12.4, 12.8, 13.8, 15.2, 18.0, 18.9, 19.3, 19.5, 21.3, and 22.4° 2θ±0.2° 2θ. In one embodiment, the diffractogram of Compound I edisylate Material A comprises at least eight of the following peaks: 9.3, 9.6, 12.4, 12.8, 13.8, 15.2, 18.0, 18.9, 19.3, 19.5, 21.3, and 22.4° 2θ±0.2° 2θ. In one embodiment, the diffractogram of Compound I edisylate Material A comprises each of the following peaks: 9.3, 9.6, 12.4, 12.8, 13.8, 15.2, 18.0, 18.9, 19.3, 19.5, 21.3, and 22.4° 2θ±0.2° 2θ. In one embodiment, Compound I edisylate Material A is characterized by the X-ray powder diffractogram as substantially shown in FIG. 48.

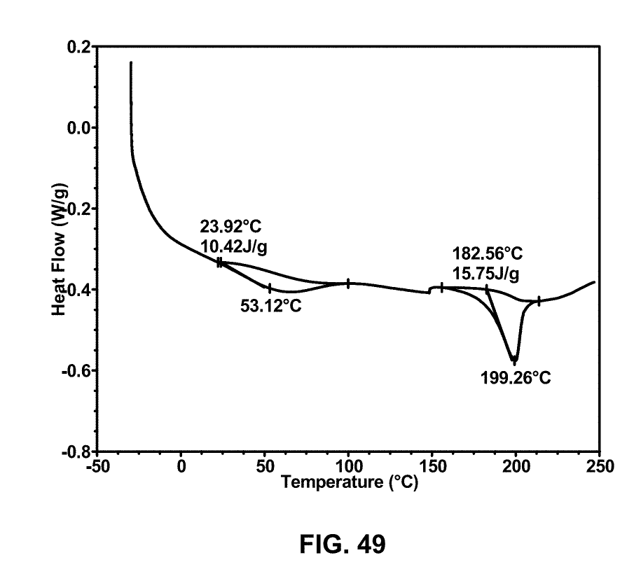

In one embodiment, Compound I edisylate Material A is characterized by a differential scanning calorimeter (DSC) curve that comprises an endotherm with onset at about 183° C. In one embodiment, the DSC curve of Compound I edisylate Material A shows an additional endotherm with onset at about 24° C. In one embodiment, Compound I edisylate Material A is characterized by the DSC curve as substantially shown in FIG. 49.

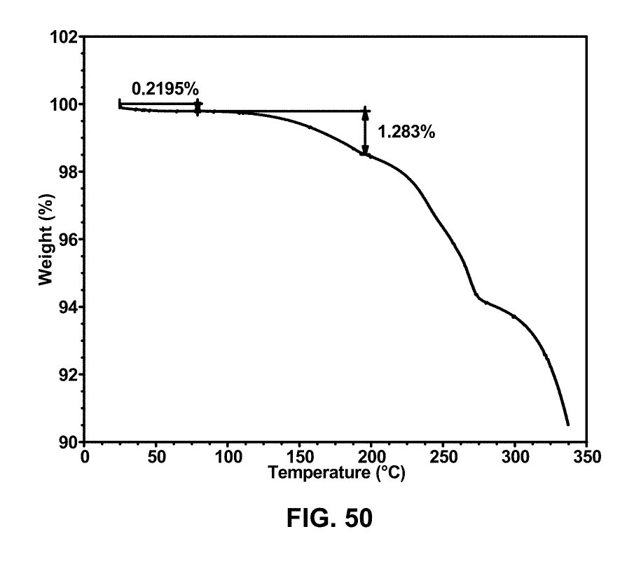

In one embodiment, Compound I edisylate Material A is characterized by a thermogravimetric analysis (TGA) thermogram showing a weight loss of about 0.2% from about 25° C. to about 79° C. In one embodiment, the TGA thermogram of Compound I edisylate Material A additionally shows a weight loss of about 1.3% from about 100° C. to about 197° C. In one embodiment, Compound I edisylate Material A is characterized by the TGA thermogram as substantially shown in FIG. 50.

In one embodiment, Compound I edisylate Material A exhibits a kinetic aqueous solubility of about 1 mg/mL.

The present disclosure also provides at least one process for making Compound I edisylate Material A. In one embodiment, the process comprises contacting Compound I with IPA and ethanedisulfonic acid (about 2 eq.), whereby Compound I edisylate Material A is formed. In one embodiment, the process for making Compound I edisylate Material A is as described in the Examples provided herein.

Compound I Besylate Material A

The present disclosure provides, in one embodiment, a besylate complex of (2-cyclopropyl-6-(3,5-dimethylisoxazol-4-yl)-1H-benzo[d]imidazol-4-yl)di(pyridin-2-yl)methanol having a crystalline form (Compound I besylate Material A). In one embodiment, Compound I besylate Material A corresponds to a besylate salt of Compound I. In one embodiment, Compound I besylate Material A corresponds to a besylate co-crystal of Compound I.

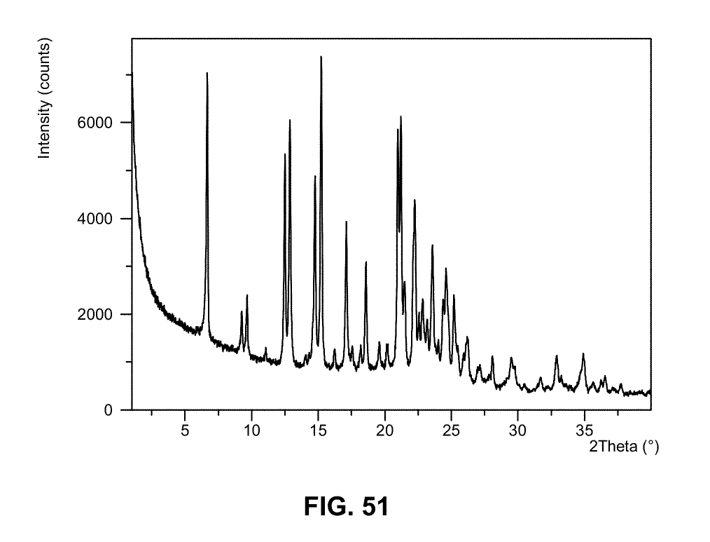

Compound I besylate Material A is characterized by an X-ray powder diffractogram comprising the following peaks: 12.5, 15.2 and 21.0° 2θ±0.2° 2θ, as determined on a diffractometer using Cu-Kα radiation at a wavelength of 1.5406 Å. In one embodiment, the diffractogram of Compound I besylate Material A further comprises one or more peaks at: 6.7, 12.9, 14.8, 17.1, 18.6, 21.2, 22.3, and 23.6° 2θ±0.2° 2θ. In one embodiment, the diffractogram of Compound I besylate Material A comprises at least two of the following peaks: 6.7, 9.3, 9.7, 12.5, 12.9, 14.8, 15.2, 17.1, 18.6, 21.0, 21.2, 22.3, and 23.6° 2θ±0.2° 2θ. In one embodiment, the diffractogram of Compound I besylate Material A comprises at least four of the following peaks: 6.7, 9.3, 9.7, 12.5, 12.9, 14.8, 15.2, 17.1, 18.6, 21.0, 21.2, 22.3, and 23.6° 2θ±0.2° 2θ. In one embodiment, the diffractogram of Compound I besylate Material A comprises at least six of the following peaks: 6.7, 9.3, 9.7, 12.5, 12.9, 14.8, 15.2, 17.1, 18.6, 21.0, 21.2, 22.3, and 23.6° 2θ±0.2° 2θ. In one embodiment, the diffractogram of Compound I besylate Material A comprises at least eight of the following peaks: 6.7, 9.3, 9.7, 12.5, 12.9, 14.8, 15.2, 17.1, 18.6, 21.0, 21.2, 22.3, and 23.6° 2θ±0.2° 2θ. In one embodiment, the diffractogram of Compound I besylate Material A comprises each of the following peaks: 6.7, 9.3, 9.7, 12.5, 12.9, 14.8, 15.2, 17.1, 18.6, 21.0, 21.2, 22.3, and 23.6° 2θ±0.2° 2θ. In one embodiment, Compound I besylate Material A is characterized by the X-ray powder diffractogram as substantially shown in FIG. 51.

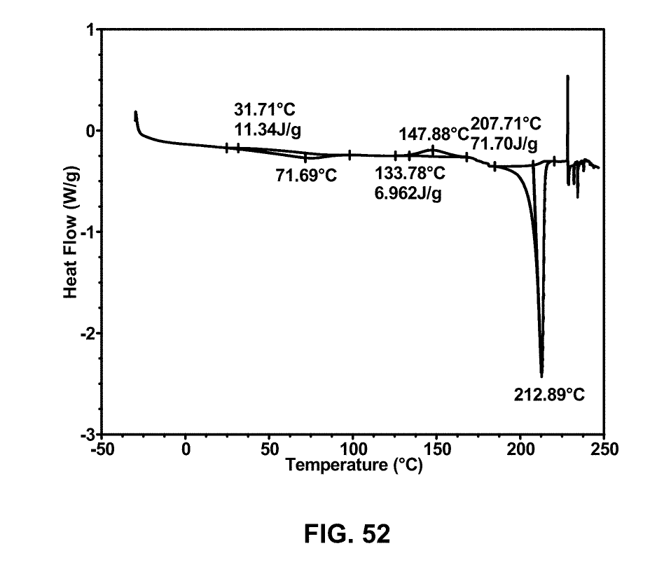

In one embodiment, Compound I besylate Material A is characterized by a differential scanning calorimeter (DSC) curve that comprises an endotherm with onset at about 208° C. In one embodiment, the DSC curve of Compound I besylate Material A shows an additional endotherm with onset at about 32° C., and an exotherm with onset at about 134° C. In one embodiment, Compound I besylate Material A is characterized by the DSC curve as substantially shown in FIG. 52.

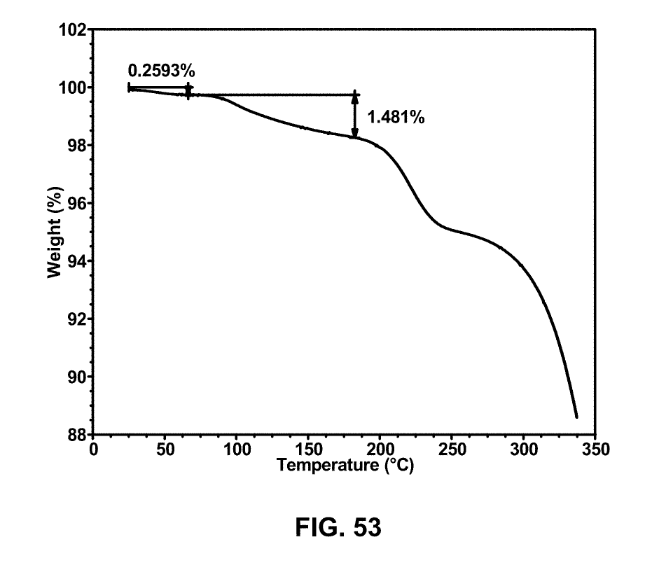

In one embodiment, Compound I besylate Material A is characterized by a thermogravimetric analysis (TGA) thermogram showing a weight loss of about 0.3% from about 25° C. to about 73° C. In one embodiment, the TGA thermogram of Compound I besylate Material A additionally shows a weight loss of about 1.5% from about 73° C. to about 182° C. In one embodiment, Compound I besylate Material A is characterized by the TGA thermogram as substantially shown in FIG. 53.

In one embodiment, Compound I besylate Material A exhibits a kinetic aqueous solubility of about 1 mg/mL.

The present disclosure also provides at least one process for making Compound I besylate Material A. In one embodiment, the process comprises contacting Compound I with EtOAc and benzenesulfonic acid (about 1 eq.), whereby Compound I besylate Material A is formed. In one embodiment, the process for making Compound I besylate Material A is as described in the Examples provided herein.

Compound I Mesylate Material A

The present disclosure provides, in one embodiment, a mesylate complex of (2-cyclopropyl-6-(3,5-dimethylisoxazol-4-yl)-1H-benzo[d]imidazol-4-yl)di(pyridin-2-yl)methanol having a crystalline form (Compound I mesylate Material A). In one embodiment, Compound I mesylate Material A corresponds to a mesylate salt of Compound I. In one embodiment, Compound I mesylate Material A corresponds to a mesylate co-crystal of Compound I.

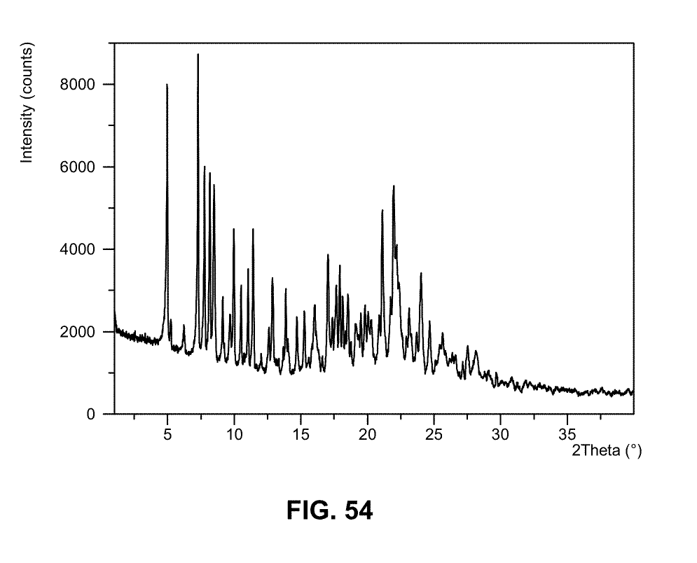

Compound I mesylate Material A is characterized by an X-ray powder diffractogram comprising the following peaks: 7.3, 10.0, and 11.4° 2θ±0.2° 2θ, as determined on a diffractometer using Cu-Kα radiation at a wavelength of 1.5406 Å. In one embodiment, the diffractogram of Compound I mesylate Material A further comprises one or more peaks at: 5.0, 7.8, 8.2, 12.9, 17.9, 21.1, and 21.9° 2θ±0.2° 2θ. In one embodiment, the diffractogram of Compound I mesylate Material A comprises at least two of the following peaks: 5.0, 7.3, 7.8, 8.2, 9.1, 9.7, 10.0, 10.5, 11.0, 11.4, 12.9, 14.7, 15.3, 16.0, 17.4, 17.7, 17.9, 18.1, 18.5, 19.5, 21.1, 21.7, and 21.9° 2θ±0.2° 2θ. In one embodiment, the diffractogram of Compound I mesylate Material A comprises at least four of the following peaks: 5.0, 7.3, 7.8, 8.2, 9.1, 9.7, 10.0, 10.5, 11.0, 11.4, 12.9, 14.7, 15.3, 16.0, 17.4, 17.7, 17.9, 18.1, 18.5, 19.5, 21.1, 21.7, and 21.9° 2θ±0.2° 2θ. In one embodiment, the diffractogram of Compound I mesylate Material A comprises at least six of the following peaks: 5.0, 7.3, 7.8, 8.2, 9.1, 9.7, 10.0, 10.5, 11.0, 11.4, 12.9, 14.7, 15.3, 16.0, 17.4, 17.7, 17.9, 18.1, 18.5, 19.5, 21.1, 21.7, and 21.9° 2θ±0.2° 2θ. In one embodiment, the diffractogram of Compound I mesylate Material A comprises at least eight of the following peaks: 5.0, 7.3, 7.8, 8.2, 9.1, 9.7, 10.0, 10.5, 11.0, 11.4, 12.9, 14.7, 15.3, 16.0, 17.4, 17.7, 17.9, 18.1, 18.5, 19.5, 21.1, 21.7, and 21.9° 2θ±0.2° 2θ. In one embodiment, the diffractogram of Compound I mesylate Material A comprises each of the following peaks: 5.0, 7.3, 7.8, 8.2, 9.1, 9.7, 10.0, 10.5, 11.0, 11.4, 12.9, 14.7, 15.3, 16.0, 17.4, 17.7, 17.9, 18.1, 18.5, 19.5, 21.1, 21.7, and 21.9° 2θ±0.2° 2θ. In one embodiment, Compound I mesylate Material A is characterized by the X-ray powder diffractogram as substantially shown in FIG. 54, which includes the presence of Compound I free base.

In one embodiment, Compound I mesylate Material A exhibits a kinetic aqueous solubility of about 5 mg/mL.

The present disclosure also provides at least one process for making Compound I mesylate Material A. In one embodiment, the process comprises contacting Compound I with 2-MeTHF and methanesulfonic acid (about 1 eq.), whereby Compound I mesylate Material A is formed. In one embodiment, the process for making Compound I mesylate Material A is as described in the Examples provided herein.

Compound I Mesylate Material B

The present disclosure provides, in one embodiment, a mesylate complex of (2-cyclopropyl-6-(3,5-dimethylisoxazol-4-yl)-1H-benzo[d]imidazol-4-yl)di(pyridin-2-yl)methanol having a crystalline form (Compound I mesylate Material B). In one embodiment, Compound I mesylate Material B corresponds to a mesylate salt of Compound I. In one embodiment, Compound I mesylate Material B corresponds to a mesylate co-crystal of Compound I.

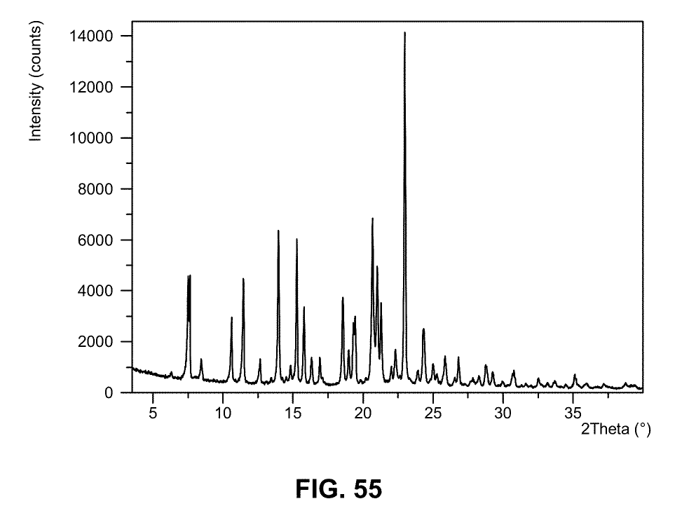

Compound I mesylate Material B is characterized by an X-ray powder diffractogram comprising the following peaks: 7.5, 20.7, and 23.0° 2θ±0.2° 2θ, as determined on a diffractometer using Cu-Kα radiation at a wavelength of 1.5406 Å. In one embodiment, the diffractogram of Compound I mesylate Material B further comprises one or more peaks at: 10.6, 11.5, 14.0, 15.3, 18.6, 21.0, and 24.3° 2θ±0.2° 2θ. In one embodiment, the diffractogram of Compound I mesylate Material B comprises at least two of the following peaks: 7.5, 7.6, 10.6, 11.5, 14.0, 15.3, 15.8, 18.6, 19.4, 20.7, 21.0, 21.3, 23.0, and 24.3° 2θ±0.2° 2θ. In one embodiment, the diffractogram of Compound I mesylate Material B comprises at least four of the following peaks: 7.5, 7.6, 10.6, 11.5, 14.0, 15.3, 15.8, 18.6, 19.4, 20.7, 21.0, 21.3, 23.0, and 24.3° 2θ±0.2° 2θ. In one embodiment, the diffractogram of Compound I mesylate Material B comprises at least six of the following peaks: 7.5, 7.6, 10.6, 11.5, 14.0, 15.3, 15.8, 18.6, 19.4, 20.7, 21.0, 21.3, 23.0, and 24.3° 2θ±0.2° 2θ. In one embodiment, the diffractogram of Compound I mesylate Material B comprises at least eight of the following peaks: 7.5, 7.6, 10.6, 11.5, 14.0, 15.3, 15.8, 18.6, 19.4, 20.7, 21.0, 21.3, 23.0, and 24.3° 2θ±0.2° 2θ. In one embodiment, the diffractogram of Compound I mesylate Material B comprises each of the following peaks: 7.5, 7.6, 10.6, 11.5, 14.0, 15.3, 15.8, 18.6, 19.4, 20.7, 21.0, 21.3, 23.0, and 24.3° 2θ±0.2° 2θ. In one embodiment, Compound I mesylate Material B is characterized by the X-ray powder diffractogram as substantially shown in FIG. 55.

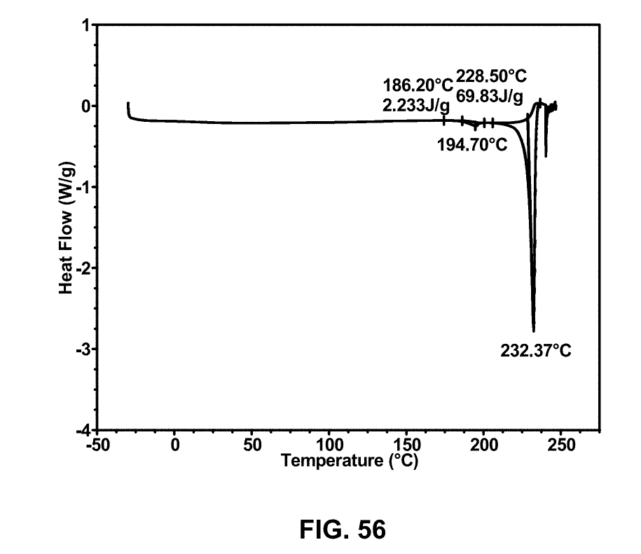

In one embodiment, Compound I mesylate Material B is characterized by a differential scanning calorimeter (DSC) curve that comprises an endotherm with onset at about 229° C. In one embodiment, the DSC curve of Compound I mesylate Material B shows an additional endotherm with onset at about 186° C. In one embodiment, Compound I mesylate Material B is characterized by the DSC curve as substantially shown in FIG. 56.

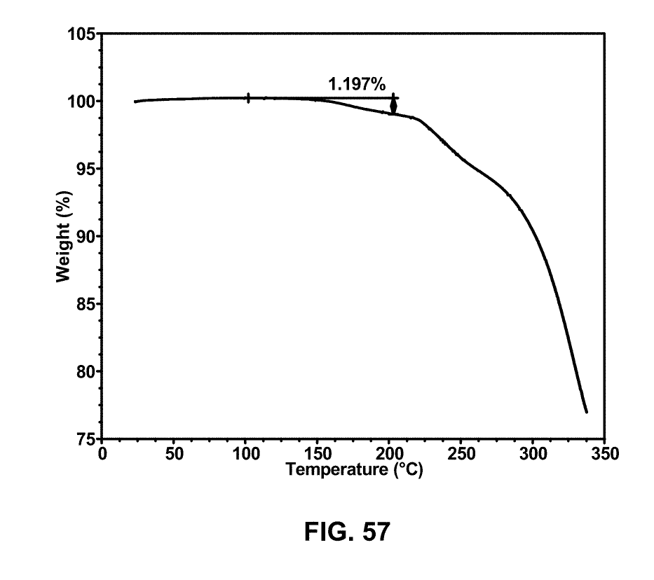

In one embodiment, Compound I mesylate Material B is characterized by a thermogravimetric analysis (TGA) thermogram showing no weight loss prior to about 130° C. In one embodiment, the TGA thermogram of Compound I mesylate Material B additionally shows a weight loss of about 1.2% from about 132° C. to about 206° C. In one embodiment, Compound I mesylate Material B is characterized by the TGA thermogram as substantially shown in FIG. 57.

The present disclosure also provides at least one process for making Compound I mesylate Material B. In one embodiment, the process comprises contacting Compound I with toluene, IPAc, and methanesulfonic acid (about 1 eq.), whereby Compound I mesylate Material B is formed. In one embodiment, the process for making Compound I mesylate Material B is as described in the Examples provided herein.

Compound I Mesylate Material C

The present disclosure provides, in one embodiment, a mesylate complex of (2-cyclopropyl-6-(3,5-dimethylisoxazol-4-yl)-1H-benzo[d]imidazol-4-yl)di(pyridin-2-yl)methanol having a crystalline form (Compound I mesylate Material C). In one embodiment, Compound I mesylate Material C corresponds to a mesylate salt of Compound I. In one embodiment, Compound I mesylate Material C corresponds to a mesylate co-crystal of Compound I.

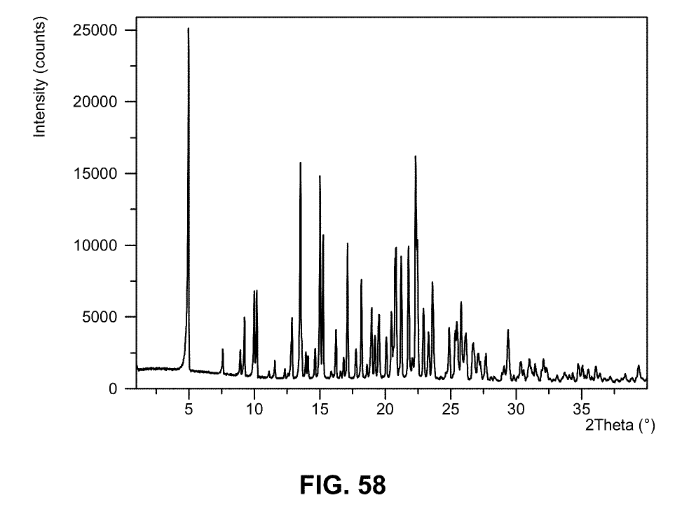

Compound I mesylate Material C is characterized by an X-ray powder diffractogram comprising the following peaks: 5.0, 13.5, and 15.0° 2θ±0.2° 2θ, as determined on a diffractometer using Cu-Kα radiation at a wavelength of 1.5406 Å. In one embodiment, the diffractogram of Compound I mesylate Material C further comprises one or more peaks at: 9.3, 10.0, 10.2, 17.1, 18.2, 20.8, 21.2, 21.8, 22.3, 23.6, 25.8, and 29.4° 2θ±0.2° 2θ. In one embodiment, the diffractogram of Compound I mesylate Material C comprises at least two of the following peaks: 5.0, 9.3, 10.0, 10.2, 12.9, 13.5, 15.0, 15.3, 17.1, 18.2, 19.0, 19.5, 20.8, 21.2, 21.8, 22.3, 22.9, 23.6, 24.9, 25.5, 25.8, and 29.4° 2θ±0.2° 2θ. In one embodiment, the diffractogram of Compound I mesylate Material C comprises at least four of the following peaks: 5.0, 9.3, 10.0, 10.2, 12.9, 13.5, 15.0, 15.3, 17.1, 18.2, 19.0, 19.5, 20.8, 21.2, 21.8, 22.3, 22.9, 23.6, 24.9, 25.5, 25.8, and 29.4° 2θ±0.2° 2θ. In one embodiment, the diffractogram of Compound I mesylate Material C comprises at least six of the following peaks: 5.0, 9.3, 10.0, 10.2, 12.9, 13.5, 15.0, 15.3, 17.1, 18.2, 19.0, 19.5, 20.8, 21.2, 21.8, 22.3, 22.9, 23.6, 24.9, 25.5, 25.8, and 29.4° 2θ±0.2° 2θ. In one embodiment, the diffractogram of Compound I mesylate Material C comprises at least eight of the following peaks: 5.0, 9.3, 10.0, 10.2, 12.9, 13.5, 15.0, 15.3, 17.1, 18.2, 19.0, 19.5, 20.8, 21.2, 21.8, 22.3, 22.9, 23.6, 24.9, 25.5, 25.8, and 29.4° 2θ±0.2° 2θ. In one embodiment, the diffractogram of Compound I mesylate Material C comprises each of the following peaks: 5.0, 9.3, 10.0, 10.2, 12.9, 13.5, 15.0, 15.3, 17.1, 18.2, 19.0, 19.5, 20.8, 21.2, 21.8, 22.3, 22.9, 23.6, 24.9, 25.5, 25.8, and 29.4° 2θ±0.2° 2θ. In one embodiment, Compound I mesylate Material C is characterized by the X-ray powder diffractogram as substantially shown in FIG. 58.

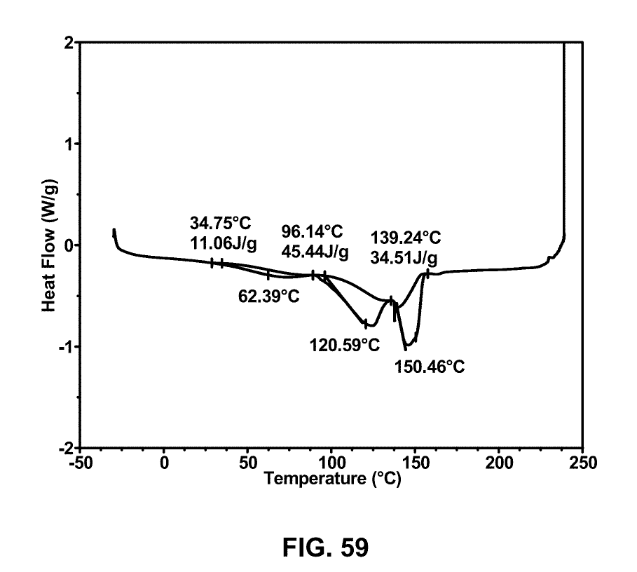

In one embodiment, Compound I mesylate Material C is characterized by a differential scanning calorimeter (DSC) curve that comprises an endotherm with onset at about 139° C. In one embodiment, the DSC curve of Compound I mesylate Material C shows additional endotherms with onsets at about 35° C. and 96° C. In one embodiment, Compound I mesylate Material C is characterized by the DSC curve as substantially shown in FIG. 59.

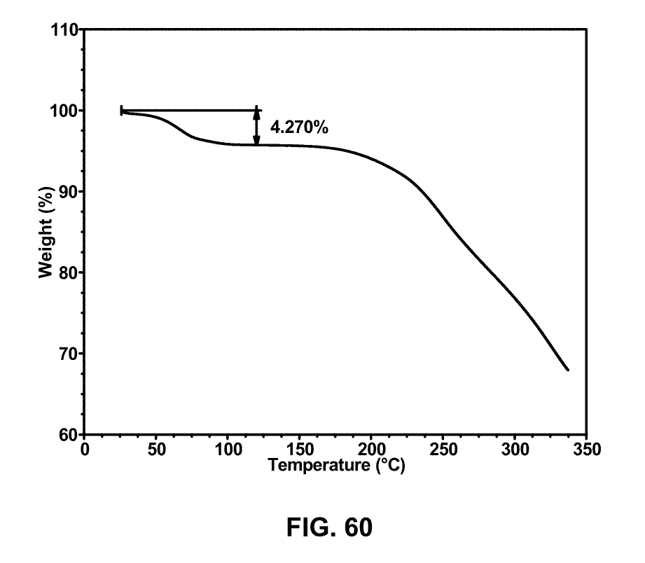

In one embodiment, Compound I mesylate Material C is characterized by a thermogravimetric analysis (TGA) thermogram showing a weight loss of about 4.3% weight loss below about 110° C. In one embodiment, Compound I mesylate Material C is characterized by the TGA thermogram as substantially shown in FIG. 60.

In one embodiment, Compound I mesylate Material C is characterized as a monohydrate. In one embodiment, Compound I mesylate Material C exhibits aqueous solubility of about 13 mg/mL.

The present disclosure also provides at least one process for making Compound I mesylate Material B. In one embodiment, the process comprises contacting Compound I with 2-MeTHF and methanesulfonic acid (about 3 eq.), whereby Compound I mesylate Material C is formed. In one embodiment, the process for making Compound I mesylate Material C is as described in the Examples provided herein.

Compound I Mesylate Material D

The present disclosure provides, in one embodiment, a mesylate complex of (2-cyclopropyl-6-(3,5-dimethylisoxazol-4-yl)-1H-benzo[d]imidazol-4-yl)di(pyridin-2-yl)methanol having a crystalline form (Compound I mesylate Material D). In one embodiment, Compound I mesylate Material D corresponds to a mesylate salt of Compound I. In one embodiment, Compound I mesylate Material D corresponds to a mesylate co-crystal of Compound I.

Compound I mesylate Material D is characterized by an X-ray powder diffractogram comprising the following peaks: 9.8, 12.9, and 17.5° 2θ±0.2° 2θ, as determined on a diffractometer using Cu-Kα radiation at a wavelength of 1.5406 Å. In one embodiment, the diffractogram of Compound I mesylate Material D further comprises one or more peaks at: 11.8, 16.8, 18.8, and 22.0° 2θ±0.2° 2θ. In one embodiment, the diffractogram of Compound I mesylate Material D comprises at least two of the following peaks: 9.8, 11.8, 12.9, 16.8, 17.5, 18.8, and 22.0° 2θ±0.2° 2θ. In one embodiment, the diffractogram of Compound I mesylate Material D comprises at least four of the following peaks: 9.8, 11.8, 12.9, 16.8, 17.5, 18.8, and 22.0° 2θ±0.2° 2θ. In one embodiment, the diffractogram of Compound I mesylate Material D comprises at least eight of the following peaks: 9.8, 11.8, 12.9, 16.8, 17.5, 18.8, and 22.0° 2θ±0.2° 2θ. In one embodiment, the diffractogram of Compound I mesylate Material D comprises each of the following peaks: 9.8, 11.8, 12.9, 16.8, 17.5, 18.8, and 22.0° 2θ±0.2° 2θ.

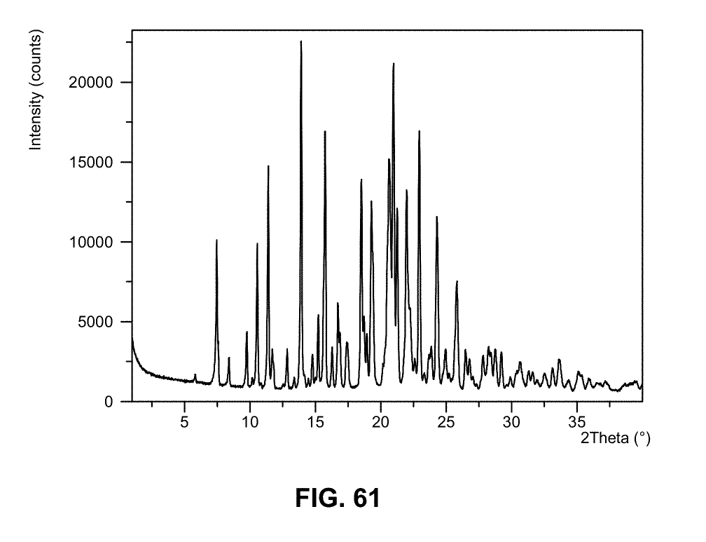

In one embodiment, Compound I mesylate Material D is present as a mixture with Compound I mesylate Material B. In one embodiment, Compound I mesylate Material D is characterized by the X-ray powder diffractogram as substantially shown in FIG. 61, which includes the presence of Compound I mesylate Material B.

The present disclosure also provides at least one process for making Compound I mesylate Material D. In one embodiment, the process comprises contacting Compound I with IPAc and methanesulfonic acid (about 1 eq.), whereby Compound I mesylate Material D is formed as a mixture with Compound I mesylate Material B. In one embodiment, the process for making Compound I mesylate Material D is as described in the Examples provided herein.

Compound I Mesylate Material E

The present disclosure provides, in one embodiment, a mesylate complex of (2-cyclopropyl-6-(3,5-dimethylisoxazol-4-yl)-1H-benzo[d]imidazol-4-yl)di(pyridin-2-yl)methanol having a crystalline form (Compound I mesylate Material E). In one embodiment, Compound I mesylate Material E corresponds to a mesylate salt of Compound I. In one embodiment, Compound I mesylate Material E corresponds to a mesylate co-crystal of Compound I.

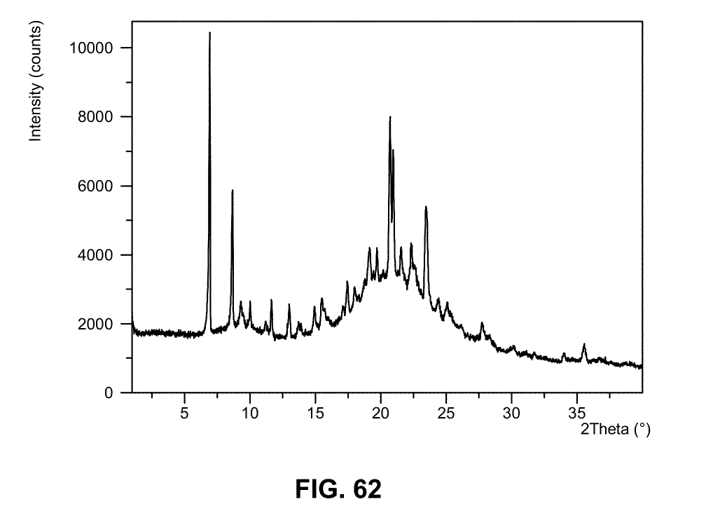

Compound I mesylate Material E is characterized by an X-ray powder diffractogram comprising the following peaks: 6.9, 8.7, and 20.7° 2θ±0.2° 2θ, as determined on a diffractometer using Cu-Kα radiation at a wavelength of 1.5406 Å. In one embodiment, the diffractogram of Compound I mesylate Material E further comprises one or more peaks at: 9.3, 10.0, 11.7, 13.0, 17.5, 20.9, and 23.4° 2θ±0.2° 2θ. In one embodiment, the diffractogram of Compound I mesylate Material E comprises at least two of the following peaks: 6.9, 8.7, 9.3, 10.0, 11.7, 13.0, 14.9, 15.5, 17.5, 19.2, 19.7, 20.7, 20.9, 21.6, 22.3, 23.4, and 23.5° 2θ±0.2° 2θ. In one embodiment, the diffractogram of Compound I mesylate Material E comprises at least four of the following peaks: 6.9, 8.7, 9.3, 10.0, 11.7, 13.0, 14.9, 15.5, 17.5, 19.2, 19.7, 20.7, 20.9, 21.6, 22.3, 23.4, and 23.5° 2θ±0.2° 2θ. In one embodiment, the diffractogram of Compound I mesylate Material E comprises at least six of the following peaks: 6.9, 8.7, 9.3, 10.0, 11.7, 13.0, 14.9, 15.5, 17.5, 19.2, 19.7, 20.7, 20.9, 21.6, 22.3, 23.4, and 23.5° 2θ±0.2° 2θ. In one embodiment, the diffractogram of Compound I mesylate Material E comprises at least eight of the following peaks: 6.9, 8.7, 9.3, 10.0, 11.7, 13.0, 14.9, 15.5, 17.5, 19.2, 19.7, 20.7, 20.9, 21.6, 22.3, 23.4, and 23.5° 2θ±0.2° 2θ. In one embodiment, the diffractogram of Compound I mesylate Material E comprises each of the following peaks: 6.9, 8.7, 9.3, 10.0, 11.7, 13.0, 14.9, 15.5, 17.5, 19.2, 19.7, 20.7, 20.9, 21.6, 22.3, 23.4, and 23.5° 2θ±0.2° 2θ. In one embodiment, Compound I mesylate Material E is characterized by the X-ray powder diffractogram as substantially shown in FIG. 62, which includes the presence of amorphous material.

The present disclosure also provides at least one process for making Compound I mesylate Material E. In one embodiment, the process comprises contacting Compound I with 2-MeTHF and methanesulfonic acid (about 2 eq.), whereby Compound I mesylate Material E is formed along with some amorphous material. In one embodiment, the process for making Compound I mesylate Material E is as described in the Examples provided herein.

Compound I Mesylate Material F

The present disclosure provides, in one embodiment, a mesylate complex of (2-cyclopropyl-6-(3,5-dimethylisoxazol-4-yl)-1H-benzo[d]imidazol-4-yl)di(pyridin-2-yl)methanol having a crystalline form (Compound I mesylate Material F). In one embodiment, Compound I mesylate Material F corresponds to a mesylate salt of Compound I. In one embodiment, Compound I mesylate Material F corresponds to a mesylate co-crystal of Compound I.

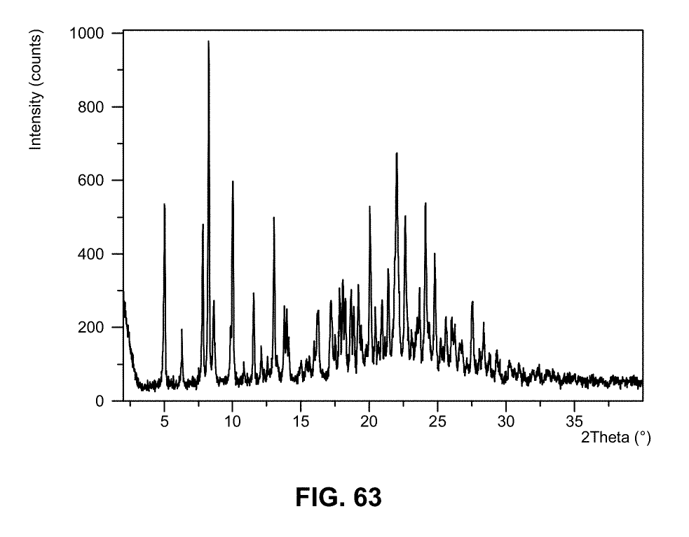

Compound I mesylate Material F is characterized by an X-ray powder diffractogram comprising the following peaks: 8.3, 10.0, and 22.0° 2θ±0.2° 2θ, as determined on a diffractometer using Cu-Kα radiation at a wavelength of 1.5406 Å. In one embodiment, the diffractogram of Compound I mesylate Material F further comprises one or more peaks at: 5.1, 7.8, 13.1, 18.1, 20.0, 22.6, 24.1, and 27.5° 2θ±0.2° 2θ. In one embodiment, the diffractogram of Compound I mesylate Material F comprises at least two of the following peaks: 5.1, 6.3, 7.8, 8.3, 8.6, 10.0, 11.6, 13.1, 14.0, 16.3, 17.2, 18.1, 18.7, 19.2, 20.0, 21.4, and 22.0° 2θ±0.2° 2θ. In one embodiment, the diffractogram of Compound I mesylate Material F comprises at least four of the following peaks: 5.1, 6.3, 7.8, 8.3, 8.6, 10.0, 11.6, 13.1, 14.0, 16.3, 17.2, 18.1, 18.7, 19.2, 20.0, 21.4, and 22.0° 2θ±0.2° 2θ. In one embodiment, the diffractogram of Compound I mesylate Material F comprises at least six of the following peaks: 5.1, 6.3, 7.8, 8.3, 8.6, 10.0, 11.6, 13.1, 14.0, 16.3, 17.2, 18.1, 18.7, 19.2, 20.0, 21.4, and 22.0° 2θ±0.2° 2θ. In one embodiment, the diffractogram of Compound I mesylate Material F comprises at least eight of the following peaks: 5.1, 6.3, 7.8, 8.3, 8.6, 10.0, 11.6, 13.1, 14.0, 16.3, 17.2, 18.1, 18.7, 19.2, 20.0, 21.4, and 22.0° 2θ±0.2° 2θ. In one embodiment, the diffractogram of Compound I mesylate Material F comprises each of the following peaks: 5.1, 6.3, 7.8, 8.3, 8.6, 10.0, 11.6, 13.1, 14.0, 16.3, 17.2, 18.1, 18.7, 19.2, 20.0, 21.4, and 22.0° 2θ±0.2° 2θ. In one embodiment, Compound I mesylate Material F is characterized by the X-ray powder diffractogram as substantially shown in FIG. 63.

The present disclosure also provides at least one process for making Compound I mesylate Material F. In one embodiment, the process comprises contacting Compound I with IPAc and methanesulfonic acid (about 1 eq.), whereby Compound I mesylate Material F is formed. In one embodiment, the process for making Compound I mesylate Material F is as described in the Examples provided herein.

Compound I Mesylate Material G

The present disclosure provides, in one embodiment, a mesylate complex of (2-cyclopropyl-6-(3,5-dimethylisoxazol-4-yl)-1H-benzo[d]imidazol-4-yl)di(pyridin-2-yl)methanol having a crystalline form (Compound I mesylate Material G). In one embodiment, Compound I mesylate Material G corresponds to a mesylate salt of Compound I. In one embodiment, Compound I mesylate Material G corresponds to a mesylate co-crystal of Compound I.

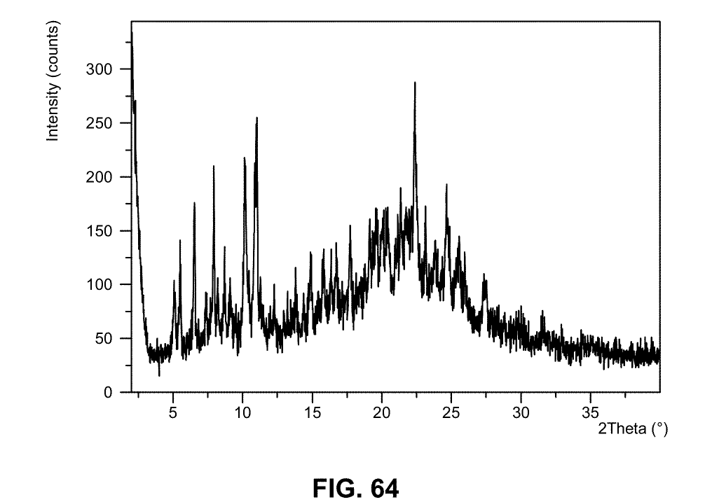

Compound I mesylate Material G is characterized by an X-ray powder diffractogram comprising the following peaks: 7.9, 11.0, and 22.4° 2θ±0.2° 2θ, as determined on a diffractometer using Cu-Kα radiation at a wavelength of 1.5406 Å. In one embodiment, the diffractogram of Compound I mesylate Material G further comprises one or more peaks at: 5.1, 5.5, 6.5, 10.2, 14.9, 17.7, 19.6, 22.4, and 24.6° 2θ±0.2° 2θ. In one embodiment, the diffractogram of Compound I mesylate Material G comprises at least two of the following peaks: 5.1, 5.5, 6.5, 7.4, 7.9, 8.7, 9.1, 10.2, 11.0, 13.8, 14.9, 15.8, 16.7, 17.7, 19.1, 19.6, 20.3, 22.4, 23.1, 24.6, 25.6, and 27.4° 2θ±0.2° 2θ. In one embodiment, the diffractogram of Compound I mesylate Material G comprises at least four of the following peaks: 5.1, 5.5, 6.5, 7.4, 7.9, 8.7, 9.1, 10.2, 11.0, 13.8, 14.9, 15.8, 16.7, 17.7, 19.1, 19.6, 20.3, 22.4, 23.1, 24.6, 25.6, and 27.4° 2θ±0.2° 2θ. In one embodiment, the diffractogram of Compound I mesylate Material G comprises at least six of the following peaks: 5.1, 5.5, 6.5, 7.4, 7.9, 8.7, 9.1, 10.2, 11.0, 13.8, 14.9, 15.8, 16.7, 17.7, 19.1, 19.6, 20.3, 22.4, 23.1, 24.6, 25.6, and 27.4° 2θ±0.2° 2θ. In one embodiment, the diffractogram of Compound I mesylate Material G comprises at least eight of the following peaks: 5.1, 5.5, 6.5, 7.4, 7.9, 8.7, 9.1, 10.2, 11.0, 13.8, 14.9, 15.8, 16.7, 17.7, 19.1, 19.6, 20.3, 22.4, 23.1, 24.6, 25.6, and 27.4° 2θ±0.2° 2θ. In one embodiment, the diffractogram of Compound I mesylate Material G comprises each of the following peaks: 5.1, 5.5, 6.5, 7.4, 7.9, 8.7, 9.1, 10.2, 11.0, 13.8, 14.9, 15.8, 16.7, 17.7, 19.1, 19.6, 20.3, 22.4, 23.1, 24.6, 25.6, and 27.4° 2θ±0.2° 2θ. In one embodiment, Compound I mesylate Material G is characterized by the X-ray powder diffractogram as substantially shown in FIG. 64, which includes the presence of amorphous material.

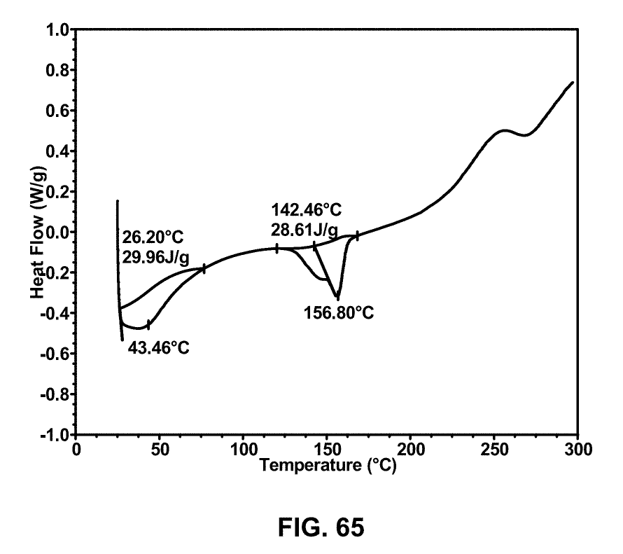

In one embodiment, Compound I mesylate Material G is characterized by a differential scanning calorimeter (DSC) curve that comprises an endotherm with onset at about 142° C. In one embodiment, Compound I mesylate Material C is characterized by a differential scanning calorimeter (DSC) curve that comprises an endotherm with onset at about 26.2° C. In one embodiment, the DSC curve of Compound I mesylate Material G shows an additional endotherm below about 80° C. In one embodiment, Compound I mesylate Material G is characterized by the DSC curve as substantially shown in FIG. 65.

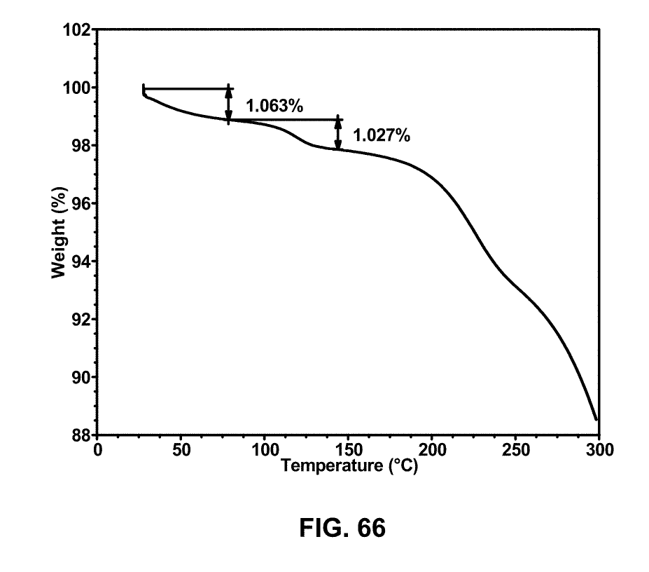

In one embodiment, Compound I mesylate Material G is characterized by a thermogravimetric analysis (TGA) thermogram showing a weight loss of about 1.1% weight loss below about 80° C. In one embodiment, the TGA thermogram of Compound I mesylate Material G additionally shows a weight loss of about 1.0% from about 80° C. to about 140° C. In one embodiment, Compound I mesylate Material G is characterized by the TGA thermogram as substantially shown in FIG. 66.

In one embodiment, Compound I mesylate Material G is characterized by dynamic vapor sorption (DVS) analysis showing a water uptake of about 25% at 90% RH.

The present disclosure also provides at least one process for making Compound I mesylate Material G. In one embodiment, the process comprises vacuum drying Compound I mesylate Form F, whereby Compound I mesylate Material G is formed along with some amorphous material. In one embodiment, the process for making Compound I mesylate Material G is as described in the Examples provided herein.

Compound I Napsylate Material A

The present disclosure provides, in one embodiment, a napsylate complex of (2-cyclopropyl-6-(3,5-dimethylisoxazol-4-yl)-1H-benzo[d]imidazol-4-yl)di(pyridin-2-yl)methanol having a crystalline form (Compound I napsylate Material A). In one embodiment, Compound I napsylate Material A corresponds to a napsylate salt of Compound I. In one embodiment, Compound I napsylate Material A corresponds to a napsylate co-crystal of Compound I.

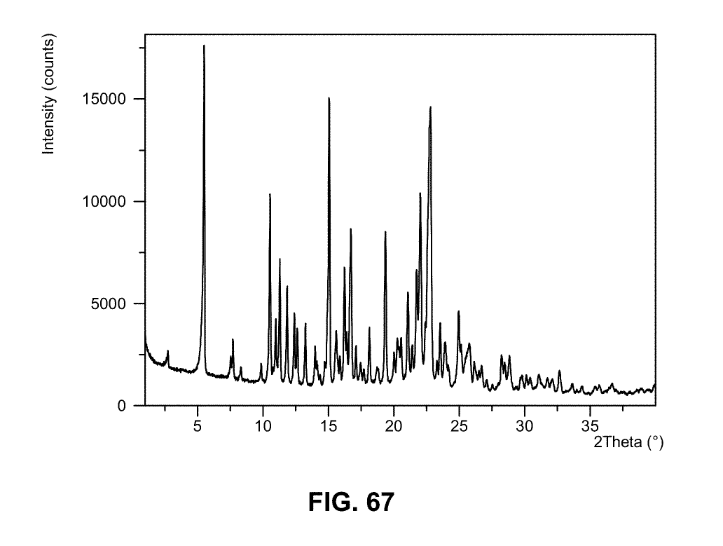

Compound I napsylate Material A is characterized by an X-ray powder diffractogram comprising the following peaks: 5.5, 15.1, and 22.8° 2θ±0.2° 2θ, as determined on a diffractometer using Cu-Kα radiation at a wavelength of 1.5406 Å. In one embodiment, the diffractogram of Compound I napsylate Material A further comprises one or more peaks at: 10.6, 11.3, 11.9, 16.7, 19.4, 22.0, and 25.0° 2θ±0.2° 2θ. In one embodiment, the diffractogram of Compound I napsylate Material A comprises at least two of the following peaks: 5.5, 7.7, 10.6, 11.0, 11.3, 11.9, 12.4, 13.3, 15.1, 16.2, 16.7, 18.1, 19.4, 21.1, 21.7, 22.0, 22.7, 22.8, and 25.0° 2θ±0.2° 2θ. In one embodiment, the diffractogram of Compound I napsylate Material A comprises at least four of the following peaks: 5.5, 7.7, 10.6, 11.0, 11.3, 11.9, 12.4, 13.3, 15.1, 16.2, 16.7, 18.1, 19.4, 21.1, 21.7, 22.0, 22.7, 22.8, and 25.0° 2θ±0.2° 2θ. In one embodiment, the diffractogram of Compound I napsylate Material A comprises at least six of the following peaks: 5.5, 7.7, 10.6, 11.0, 11.3, 11.9, 12.4, 13.3, 15.1, 16.2, 16.7, 18.1, 19.4, 21.1, 21.7, 22.0, 22.7, 22.8, and 25.0° 2θ±0.2° 2θ. In one embodiment, the diffractogram of Compound I napsylate Material A comprises at least eight of the following peaks: 5.5, 7.7, 10.6, 11.0, 11.3, 11.9, 12.4, 13.3, 15.1, 16.2, 16.7, 18.1, 19.4, 21.1, 21.7, 22.0, 22.7, 22.8, and 25.0° 2θ±0.2° 2θ. In one embodiment, the diffractogram of Compound I napsylate Material A comprises each of the following peaks: 5.5, 7.7, 10.6, 11.0, 11.3, 11.9, 12.4, 13.3, 15.1, 16.2, 16.7, 18.1, 19.4, 21.1, 21.7, 22.0, 22.7, 22.8, and 25.0° 2θ±0.2° 2θ. In one embodiment, Compound I napsylate Material A is characterized by the X-ray powder diffractogram as substantially shown in FIG. 67.

In one embodiment, Compound I napsylate Material A exhibits a kinetic aqueous solubility less than about 1 mg/mL.

The present disclosure also provides at least one process for making Compound I napsylate Material A. In one embodiment, the process comprises contacting Compound I with 2-MeTHF and naphthalenesulfonic acid (about 1 eq.), whereby Compound I napsylate Material A is formed. In one embodiment, the process for making Compound I napsylate Material A is as described in the Examples provided herein.

Compound I Tartrate Material A

The present disclosure provides, in one embodiment, a tartrate complex of (2-cyclopropyl-6-(3,5-dimethylisoxazol-4-yl)-1H-benzo[d]imidazol-4-yl)di(pyridin-2-yl)methanol having a crystalline form (Compound I tartrate Material A). In one embodiment, Compound I tartrate Material A corresponds to a tartrate salt of Compound I. In one embodiment, Compound I tartrate Material A corresponds to a tartrate co-crystal of Compound I.

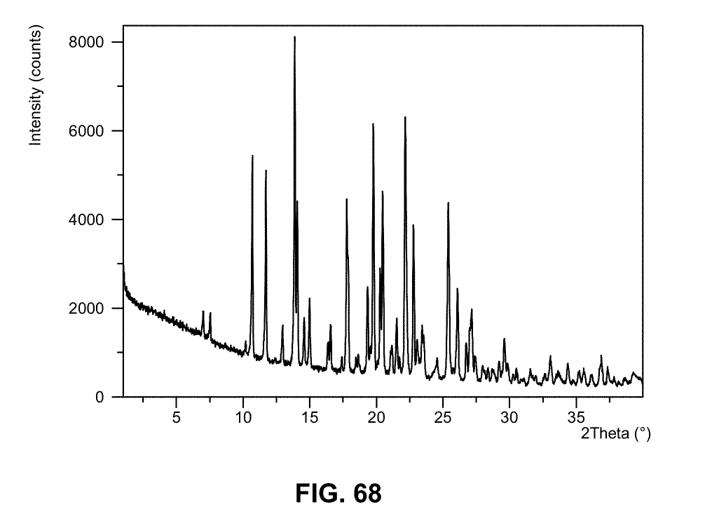

Compound I tartrate Material A is characterized by an X-ray powder diffractogram comprising the following peaks: 13.9, 19.8, and 22.2° 2θ±0.2° 2θ, as determined on a diffractometer using Cu-Kα radiation at a wavelength of 1.5406 Å. In one embodiment, the diffractogram of Compound I tartrate Material A further comprises one or more peaks at: 10.7, 11.7, 17.8, 20.5, 22.8, and 25.4° 2θ±0.2° 2θ. In one embodiment, the diffractogram of Compound I tartrate Material A comprises at least two of the following peaks: 7.0, 7.5, 10.7, 11.7, 13.0, 13.9, 14.1, 14.6, 15.0, 16.6, 17.8, 17.9, 19.3, 19.8, 20.5, 21.5, 22.2, 22.8, 25.4, 26.1, 27.2, and 29.6° 2θ±0.2° 2θ. In one embodiment, the diffractogram of Compound I tartrate Material A comprises at least four of the following peaks: 7.0, 7.5, 10.7, 11.7, 13.0, 13.9, 14.1, 14.6, 15.0, 16.6, 17.8, 17.9, 19.3, 19.8, 20.5, 21.5, 22.2, 22.8, 25.4, 26.1, 27.2, and 29.6° 2θ±0.2° 2θ. In one embodiment, the diffractogram of Compound I tartrate Material A comprises at least six of the following peaks: 7.0, 7.5, 10.7, 11.7, 13.0, 13.9, 14.1, 14.6, 15.0, 16.6, 17.8, 17.9, 19.3, 19.8, 20.5, 21.5, 22.2, 22.8, 25.4, 26.1, 27.2, and 29.6° 2θ±0.2° 2θ. In one embodiment, the diffractogram of Compound I tartrate Material A comprises at least eight of the following peaks: 7.0, 7.5, 10.7, 11.7, 13.0, 13.9, 14.1, 14.6, 15.0, 16.6, 17.8, 17.9, 19.3, 19.8, 20.5, 21.5, 22.2, 22.8, 25.4, 26.1, 27.2, and 29.6° 2θ±0.2° 2θ. In one embodiment, the diffractogram of Compound I tartrate Material A comprises each of the following peaks: 7.0, 7.5, 10.7, 11.7, 13.0, 13.9, 14.1, 14.6, 15.0, 16.6, 17.8, 17.9, 19.3, 19.8, 20.5, 21.5, 22.2, 22.8, 25.4, 26.1, 27.2, and 29.6° 2θ±0.2° 2θ. In one embodiment, Compound I tartrate Material A is characterized by the X-ray powder diffractogram as substantially shown in FIG. 68.

In one embodiment, Compound I tartrate Material A exhibits a kinetic aqueous solubility of about 15 mg/mL.

The present disclosure also provides at least one process for making Compound I tartrate Material A. In one embodiment, the process comprises contacting Compound I with EtOAc, IPA, and L-tartaric acid (about 2 eq.), whereby Compound I tartrate Material A is formed. In one embodiment, the process for making Compound I tartrate Material A is as described in the Examples provided herein.

Compound I Tartrate Material B

The present disclosure provides, in one embodiment, a tartrate complex of (2-cyclopropyl-6-(3,5-dimethylisoxazol-4-yl)-1H-benzo[d]imidazol-4-yl)di(pyridin-2-yl)methanol having a crystalline form (Compound I tartrate Material B). In one embodiment, Compound I tartrate Material B corresponds to a tartrate salt of Compound I. In one embodiment, Compound I tartrate Material B corresponds to a tartrate co-crystal of Compound I.

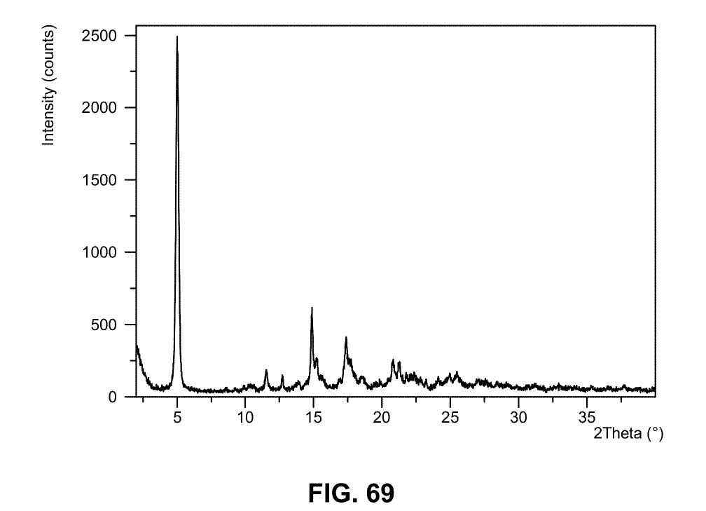

Compound I tartrate Material B is characterized by an X-ray powder diffractogram comprising the following peaks: 5.0, 14.9, and 17.4° 2θ±0.2° 2θ, as determined on a diffractometer using Cu-Kα radiation at a wavelength of 1.5406 Å. In one embodiment, the diffractogram of Compound I tartrate Material B further comprises one or more peaks at: 11.5, 12.7, 15.2, 20.8, and 21.3° 2θ±0.2° 2θ. In one embodiment, the diffractogram of Compound I tartrate Material B comprises at least two of the following peaks: 5.0, 11.5, 12.7, 14.9, 15.2, 17.4, 20.8, 21.3, 22.3, 24.1, 24.9, and 25.4° 2θ±0.2° 2θ. In one embodiment, the diffractogram of Compound I tartrate Material B comprises at least four of the following peaks: 5.0, 11.5, 12.7, 14.9, 15.2, 17.4, 20.8, 21.3, 22.3, 24.1, 24.9, and 25.4° 2θ±0.2° 2θ. In one embodiment, the diffractogram of Compound I tartrate Material B comprises at least six of the following peaks: 5.0, 11.5, 12.7, 14.9, 15.2, 17.4, 20.8, 21.3, 22.3, 24.1, 24.9, and 25.4° 2θ±0.2° 2θ. In one embodiment, the diffractogram of Compound I tartrate Material B comprises at least eight of the following peaks: 5.0, 11.5, 12.7, 14.9, 15.2, 17.4, 20.8, 21.3, 22.3, 24.1, 24.9, and 25.4° 2θ±0.2° 2θ. In one embodiment, the diffractogram of Compound I tartrate Material B comprises each of the following peaks: 5.0, 11.5, 12.7, 14.9, 15.2, 17.4, 20.8, 21.3, 22.3, 24.1, 24.9, and 25.4° 2θ±0.2° 2θ. In one embodiment, Compound I tartrate Material B is characterized by the X-ray powder diffractogram as substantially shown in FIG. 69.

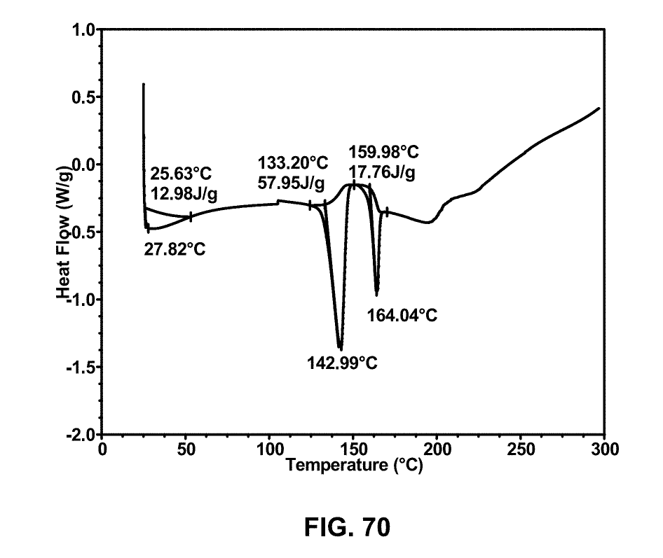

In one embodiment, Compound I tartrate Material B is characterized by a differential scanning calorimeter (DSC) curve that comprises an endotherm with onset at about 160° C. In one embodiment, the DSC curve of Compound I tartrate Material B comprises an additional endotherm with onset at about 133° C. In one embodiment, Compound I tartrate Material B is characterized by the DSC curve as substantially shown in FIG. 70.

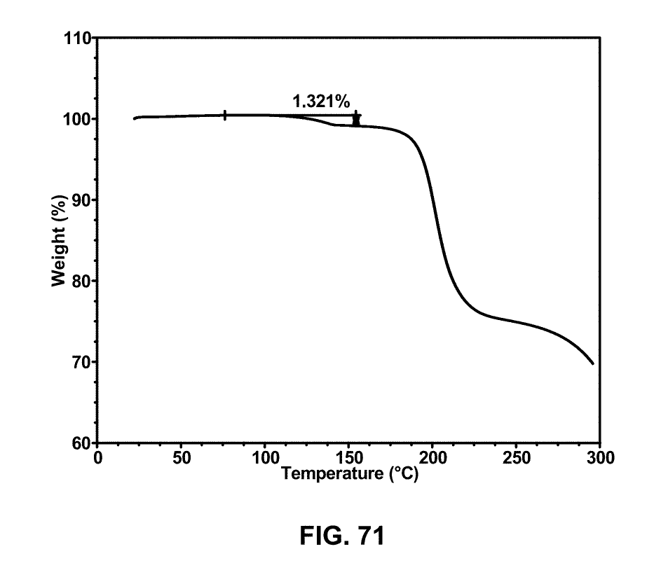

In one embodiment, Compound I tartrate Material B is characterized by a thermogravimetric analysis (TGA) thermogram showing a weight loss of about 1.3% from about 100° C. to about 150° C. In one embodiment, Compound I tartrate Material B is characterized by the TGA thermogram as substantially shown in FIG. 71.

In one embodiment, Compound I tartrate Material B is characterized as isopropanol solvate. In one embodiment, Compound I tartrate Material B comprises about 0.64% water as measured by KF analysis.

The present disclosure also provides at least one process for making Compound I tartrate Material B. In one embodiment, the process comprises contacting Compound I with IPA, and L-tartaric acid (about 1.1 eq.), whereby Compound I tartrate Material B is formed. In one embodiment, the process for making Compound I tartrate Material B is as described in the Examples provided herein.

Compound I Xinafoate Form I

The present disclosure provides, in one embodiment, a xinafoate complex of (2-cyclopropyl-6-(3,5-dimethylisoxazol-4-yl)-1H-benzo[d]imidazol-4-yl)di(pyridin-2-yl)methanol having a crystalline form (Compound I xinafoate Form I). In one embodiment, Compound I xinafoate Form I corresponds to a xinafoate salt of Compound I. In one embodiment, Compound I xinafoate Form I corresponds to a xinafoate co-crystal of Compound I.

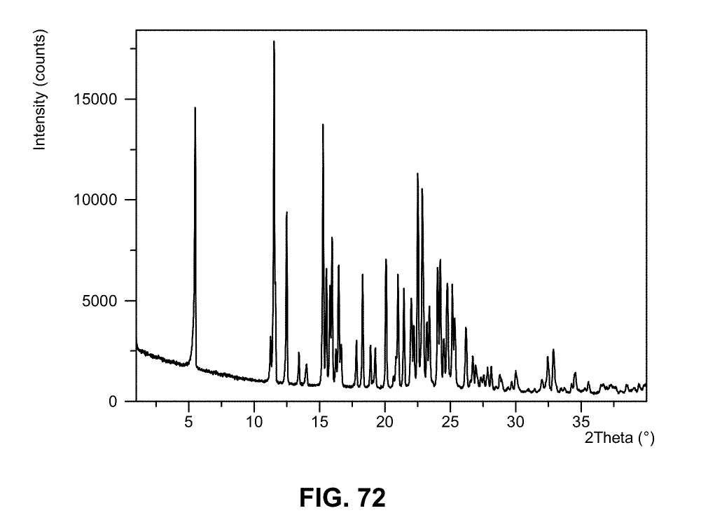

Compound I xinafoate Form I is characterized by an X-ray powder diffractogram comprising the following peaks: 5.5, 11.5, and 15.3° 2θ±0.2° 2θ, as determined on a diffractometer using Cu-Kα radiation at a wavelength of 1.5406 Å. In one embodiment, the diffractogram of Compound I xinafoate Form I further comprises one or more peaks at 12.5, 16.0, 16.5, 18.3, 20.1, 21.0, 22.5, and 22.9° 2θ±0.2° 2θ. In one embodiment, the diffractogram of Compound I xinafoate Form I comprises at least two of the following peaks: 5.5, 11.5, 12.5, 15.3, 16.0, 16.5, 18.3, 20.1, 21.0, 21.4, 22.5, 22.9, 24.0, 24.2, 24.8, 25.1, and 26.2° 2θ±0.2° 2θ. In one embodiment, the diffractogram of Compound I xinafoate Form I comprises at least four of the following peaks: 5.5, 11.5, 12.5, 15.3, 16.0, 16.5, 18.3, 20.1, 21.0, 21.4, 22.5, 22.9, 24.0, 24.2, 24.8, 25.1, and 26.2° 2θ±0.2° 2θ. In one embodiment, the diffractogram of Compound I xinafoate Form I comprises at least six of the following peaks: 5.5, 11.5, 12.5, 15.3, 16.0, 16.5, 18.3, 20.1, 21.0, 21.4, 22.5, 22.9, 24.0, 24.2, 24.8, 25.1, and 26.2° 2θ±0.2° 2θ. In one embodiment, the diffractogram of Compound I xinafoate Form I comprises at least eight of the following peaks: 5.5, 11.5, 12.5, 15.3, 16.0, 16.5, 18.3, 20.1, 21.0, 21.4, 22.5, 22.9, 24.0, 24.2, 24.8, 25.1, and 26.2° 2θ±0.2° 2θ. In one embodiment, the diffractogram of Compound I xinafoate Form I comprises each of the following peaks: 5.5, 11.5, 12.5, 15.3, 16.0, 16.5, 18.3, 20.1, 21.0, 21.4, 22.5, 22.9, 24.0, 24.2, 24.8, 25.1, and 26.2° 2θ±0.2° 2θ. In one embodiment, Compound I xinafoate Form I is characterized by the X-ray powder diffractogram as substantially shown in FIG. 72.

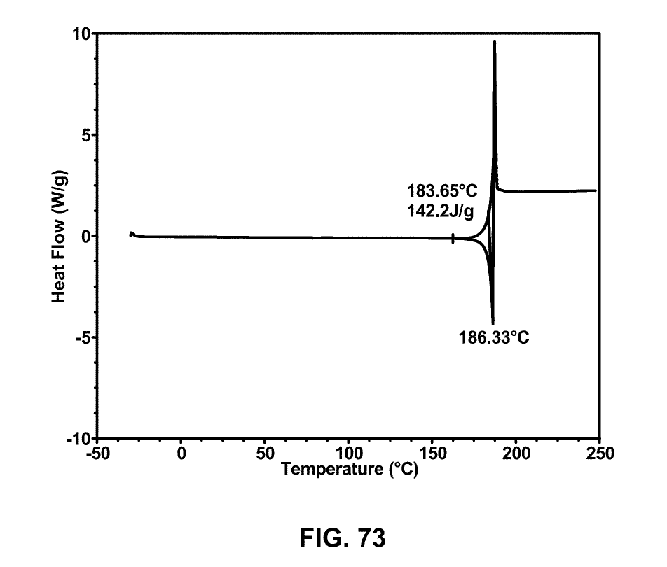

In one embodiment, Compound I xinafoate Form I is characterized by a differential scanning calorimeter (DSC) curve that comprises an endotherm with onset at about 184° C. In one embodiment, the DSC curve of Compound I xinafoate Form I comprises an exotherm immediately following the endotherm with onset at about 184° C. In one embodiment, Compound I xinafoate Form I is characterized by the DSC curve as substantially shown in FIG. 73.

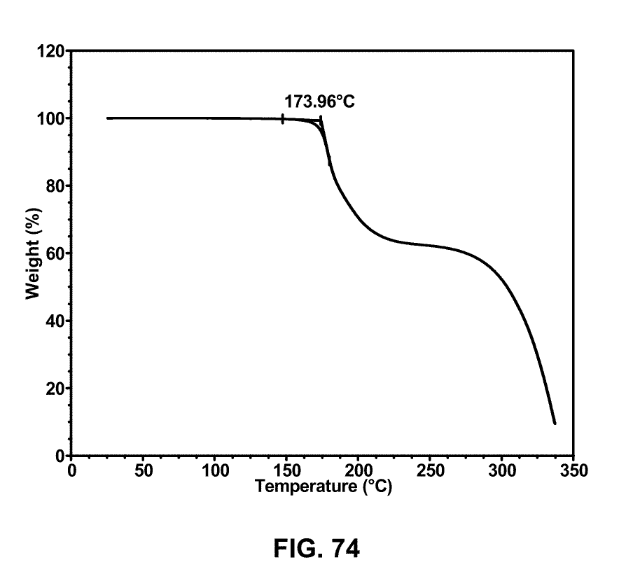

In one embodiment, Compound I xinafoate Form I is characterized by a thermogravimetric analysis (TGA) thermogram showing no weight loss prior to about 174° C. In one embodiment, Compound I xinafoate Form I is characterized by the TGA thermogram as substantially shown in FIG. 74.

In one embodiment, Compound I xinafoate Form I is characterized as anhydrous. In one embodiment, Compound I xinafoate Form I exhibits an aqueous solubility of less than about 1 mg/mL.

The present disclosure also provides at least one process for making Compound I xinafoate Form I. In one embodiment, the process comprises contacting Compound I with xinafoic acid (about 1 eq.) with ethyl acetate or a mixture of ethyl acetate and MeOH, whereby Compound I xinafoate Form I is formed. In one embodiment, the process for making Compound I xinafoate Form I is as described in the Examples provided herein.

Compound I Gentisate Material A

The present disclosure provides, in one embodiment, a gentisate complex of (2-cyclopropyl-6-(3,5-dimethylisoxazol-4-yl)-1H-benzo[d]imidazol-4-yl)di(pyridin-2-yl)methanol having a crystalline form (Compound I gentisate Material A). In one embodiment, Compound I gentisate Material A corresponds to a gentisate salt of Compound I. In one embodiment, Compound I gentisate Material A corresponds to a gentisate co-crystal of Compound I.

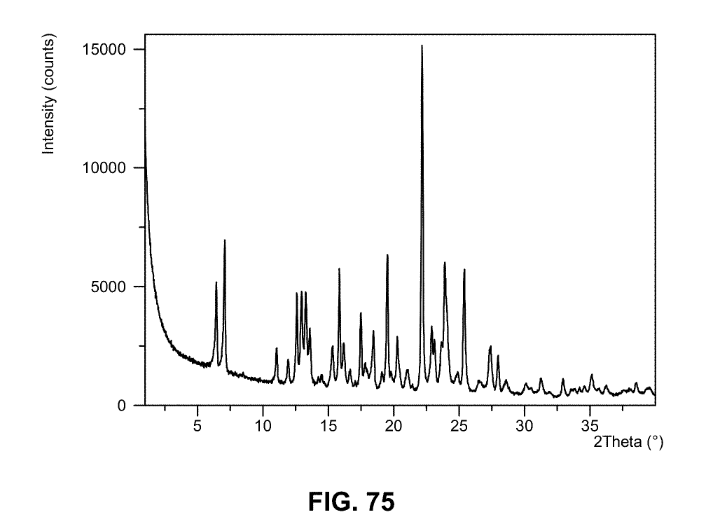

Compound I gentisate Material A is characterized by an X-ray powder diffractogram comprising the following peaks: 7.1, 19.5, and 22.2° 2θ±0.2° 2θ, as determined on a diffractometer using Cu-Kα radiation at a wavelength of 1.5406 Å. In one embodiment, the diffractogram of Compound I gentisate Material A further comprises one or more peaks at 6.5, 12.6, 13.0, 13.3, 13.6, 15.9, 17.5, 23.9 and 25.4° 2θ±0.2° 2θ. In one embodiment, the diffractogram of Compound I gentisate Material A comprises at least two of the following peaks: 6.5, 7.1, 12.6, 13.0, 13.3, 13.6, 15.3, 15.9, 16.2, 17.5, 18.5, 19.5, 20.3, 22.2, 22.9, 23.1, 23.6, 23.9, 25.4, and 27.4° 2θ±0.2° 2θ. In one embodiment, the diffractogram of Compound I gentisate Material A comprises at least four of the following peaks: 6.5, 7.1, 12.6, 13.0, 13.3, 13.6, 15.3, 15.9, 16.2, 17.5, 18.5, 19.5, 20.3, 22.2, 22.9, 23.1, 23.6, 23.9, 25.4, and 27.4° 2θ±0.2° 2θ. In one embodiment, the diffractogram of Compound I gentisate Material A comprises at least six of the following peaks: 6.5, 7.1, 12.6, 13.0, 13.3, 13.6, 15.3, 15.9, 16.2, 17.5, 18.5, 19.5, 20.3, 22.2, 22.9, 23.1, 23.6, 23.9, 25.4, and 27.4° 2θ±0.2° 2θ. In one embodiment, the diffractogram of Compound I gentisate Material A comprises at least eight of the following peaks: 6.5, 7.1, 12.6, 13.0, 13.3, 13.6, 15.3, 15.9, 16.2, 17.5, 18.5, 19.5, 20.3, 22.2, 22.9, 23.1, 23.6, 23.9, 25.4, and 27.4° 2θ±0.2° 2θ. In one embodiment, the diffractogram of Compound I gentisate Material A comprises each of the following peaks: 6.5, 7.1, 12.6, 13.0, 13.3, 13.6, 15.3, 15.9, 16.2, 17.5, 18.5, 19.5, 20.3, 22.2, 22.9, 23.1, 23.6, 23.9, 25.4, and 27.4° 2θ±0.2° 2θ. In one embodiment, Compound I gentisate Material A is characterized by the X-ray powder diffractogram as substantially shown in FIG. 75.

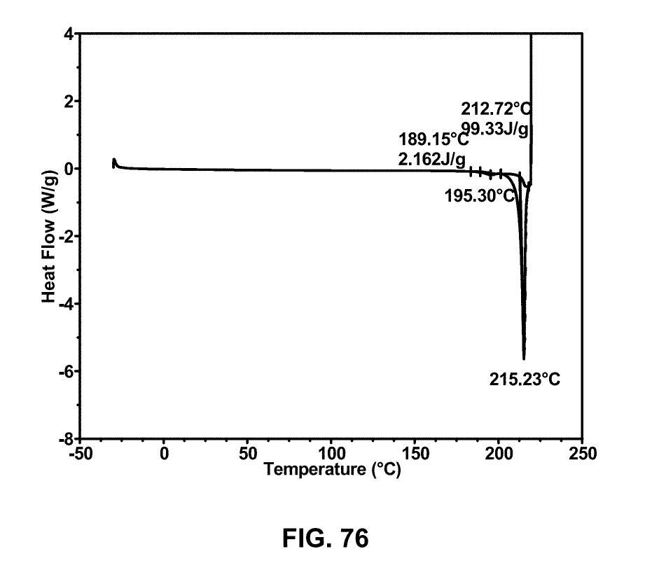

In one embodiment, Compound I gentisate Material A is characterized by a differential scanning calorimeter (DSC) curve that comprises an endotherm with onset at about 213° C. In one embodiment, the DSC curve of Compound I gentisate Material A comprises an additional endotherm with onset at about 189° C., and an exotherm at above 215° C. In one embodiment, Compound I xinafoate Form I is characterized by the DSC curve as substantially shown in FIG. 76.

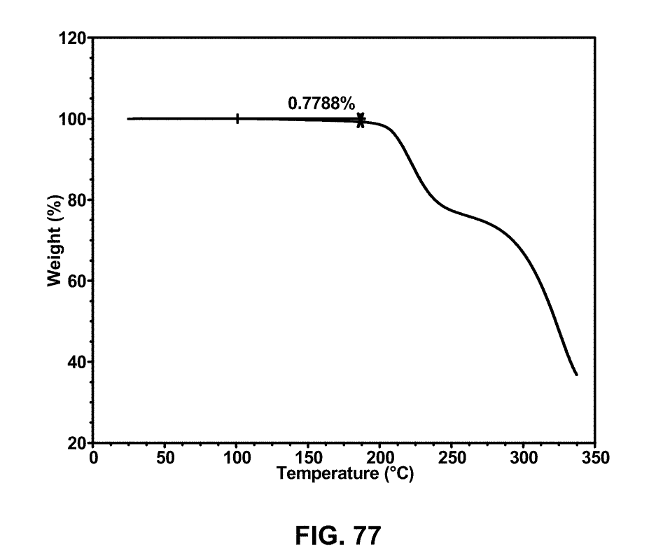

In one embodiment, Compound I gentisate Material A is characterized by a thermogravimetric analysis (TGA) thermogram showing no weight loss below about 100° C. In one embodiment, the TGA thermogram of Compound I gentisate Material A additionally shows a weight loss of about 0.8% from about 100° C. to about 190° C. In one embodiment, Compound I gentisate Material A is characterized by the TGA thermogram as substantially shown in FIG. 77.

In one embodiment, Compound I gentisate Material A is characterized as anhydrous. In one embodiment, Compound I gentisate Material A exhibits an aqueous solubility of less than about 1 mg/mL

The present disclosure also provides at least one process for making Compound I gentisate Material A. In one embodiment, the process comprises contacting Compound I with EtOAc and gentisic acid (about 1 eq.), whereby Compound I gentisate Material A is formed. In one embodiment, the process for making Compound I gentisate Material A is as described in the Examples provided herein.

Compound I Oxalate (Disordered)

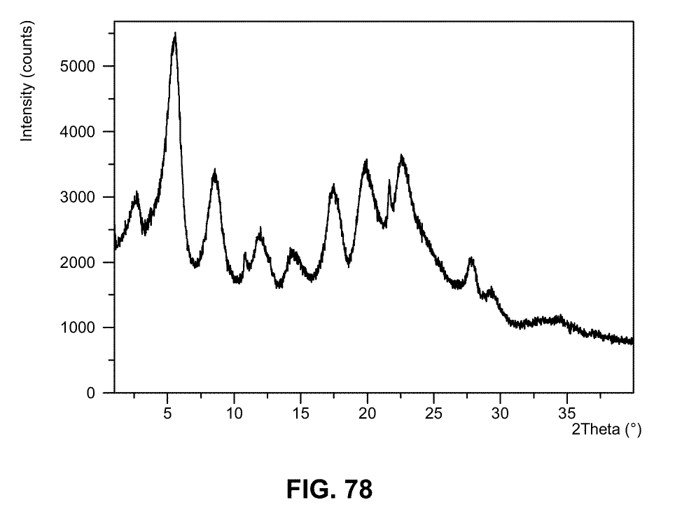

A disordered oxalate complex (e.g., oxalate salt or co-crystal) of (2-cyclopropyl-6-(3,5-dimethylisoxazol-4-yl)-1H-benzo[d]imidazol-4-yl)di(pyridin-2-yl)methanol (Compound I oxalate (disordered)) is characterized by an X-ray powder diffractogram comprising the following peaks: 5.6, 14.3, and 22.5° 2θ±0.2° 2θ, as determined on a diffractometer using Cu-Kα radiation at a wavelength of 1.5406 Å. In one embodiment, the diffractogram of Compound I oxalate (disordered) further comprises one or more peaks at 8.4, 11.9, 17.2, 19.7, and 21.7° 2θ±0.2° 2θ. In one embodiment, the diffractogram of Compound I oxalate (disordered) comprises at least two of the following peaks: 5.6, 8.4, 11.9, 14.3, 17.2, 19.7, 21.7, and 22.5° 2θ±0.2° 2θ. In one embodiment, the diffractogram of Compound I oxalate (disordered) comprises at least four of the following peaks: 5.6, 8.4, 11.9, 14.3, 17.2, 19.7, 21.7, and 22.5° 2θ±0.2° 2θ. In one embodiment, the diffractogram of Compound I oxalate (disordered) comprises at least six of the following peaks: 5.6, 8.4, 11.9, 14.3, 17.2, 19.7, 21.7, and 22.5° 2θ±0.2° 2θ. In one embodiment, the diffractogram of Compound I oxalate (disordered) comprises each of the following peaks: 5.6, 8.4, 11.9, 14.3, 17.2, 19.7, 21.7, and 22.5° 2θ±0.2° 2θ. In one embodiment, Compound I oxalate (disordered) is characterized by the X-ray powder diffractogram as substantially shown in FIG. 78.

The present disclosure also provides at least one process for making Compound I oxalate (disordered). In one embodiment, the process comprises contacting Compound I with IPA and oxalic acid whereby Compound I oxalate (disordered) is formed. In one embodiment, the process for making Compound I oxalate (disordered) is as described in the Examples provided herein.

### Pharmaceutical Compositions and Modes of Administration

The forms of Compound I as described herein may be administered in a pharmaceutical composition. Thus, provided herein are pharmaceutical compositions comprising one or more of the forms of Compound I described herein and one or more pharmaceutically acceptable vehicles such as carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.). The pharmaceutical compositions may be administered alone or in combination with other therapeutic agents.

Some embodiments are directed to pharmaceutical compositions comprising a crystalline form of Compound I as described herein. In one embodiment, a pharmaceutical composition comprises Compound I, wherein at least 95% of Compound I is in a crystalline form as described herein. In one embodiment, a pharmaceutical composition comprises Compound I, wherein at least 95% of Compound I is in Form I. In one embodiment, a pharmaceutical composition comprises Compound I, wherein at least 95% of Compound I is in Form II. In one embodiment, a pharmaceutical composition comprises Compound I, wherein at least 95% of Compound I is Compound I Material A.

In one embodiment, a pharmaceutical composition comprises Compound I, wherein at least 97% of Compound I is in a crystalline form as described herein. In one embodiment, a pharmaceutical composition comprises Compound I, wherein at least 97% of Compound I is in Form I. In one embodiment, a pharmaceutical composition comprises Compound I, wherein at least 97% of Compound I is in Form II. In one embodiment, a pharmaceutical composition comprises Compound I, wherein at least 97% of Compound I is Compound I Material A.

In one embodiment, a pharmaceutical composition comprises Compound I, wherein at least 99% of Compound I is in a crystalline form as described herein. In one embodiment, a pharmaceutical composition comprises Compound I, wherein at least 99% of Compound I is in Form I. In one embodiment, a pharmaceutical composition comprises Compound I, wherein at least 99% of Compound I is in Form II. In one embodiment, a pharmaceutical composition comprises Compound I, wherein at least 99% of Compound I is Compound I Material A.

Some embodiments are directed to pharmaceutical compositions comprising an amorphous form of Compound I as described herein. In one embodiment, a pharmaceutical composition comprises Compound I, where at least 95% of Compound I is in an amorphous form as described herein. In one embodiment, a pharmaceutical composition comprises Compound I, where at least 97% of Compound I is in an amorphous form as described herein. In one embodiment, a pharmaceutical composition comprises Compound I, wherein at least 99% of Compound I is in an amorphous form as described herein.

Some embodiments are directed to pharmaceutical compositions comprising a phosphate complex of Compound I in a crystalline form as described herein. In one embodiment, a pharmaceutical composition comprises a phosphate complex of Compound I, wherein at least 95% of the phosphate complex of Compound I is in Form I as described herein. In one embodiment, a pharmaceutical composition comprises a phosphate complex of Compound I, where at least 97% of the phosphate complex of Compound I is in Form I as described herein. In one embodiment, a pharmaceutical composition comprises a phosphate complex of Compound I, where at least 99% of the phosphate complex of Compound I is in Form I as described herein.

Some embodiments are directed to pharmaceutical compositions comprising a phosphate complex of Compound I in an amorphous form as described herein. In one embodiment, a pharmaceutical composition comprises a phosphate complex of Compound I, wherein at least 95% of the phosphate complex of Compound I is in an amorphous form as described herein. In one embodiment, a pharmaceutical composition comprises a phosphate complex of Compound I, where at least 97% of the phosphate complex of Compound I is in an amorphous form as described herein. In one embodiment, a pharmaceutical composition comprises a phosphate complex of Compound I, where at least 99% of the phosphate complex of Compound I is in as amorphous form as described herein.

Some embodiments are directed pharmaceutical compositions comprising a therapeutically effective amount of a compound selected from: Compound I Form I; Compound I Form II; Compound I Material A; Compound I amorphous; Compound I besylate Material A, Compound I edisylate Form I, Compound I gentisate Material A, Compound I HCl Material A, Compound I HCl Material B, Compound I HCl Material C, Compound I HCl Material D, Compound I HCl Material E, Compound I mesylate Material A, Compound I mesylate Material B, Compound I mesylate Material C, Compound I mesylate Material D, Compound I mesylate Material E, Compound I mesylate Material F, Compound I mesylate Material G, Compound I napsylate Material A, Compound I oxalate (disordered), Compound I phosphate Form I, Compound I phosphate Form II, Compound I phosphate Form III, Compound I phosphate Form IV, Compound I phosphate Form V, Compound I phosphate (amorphous), Compound I sulfate Material A, Compound I sulfate Material B, Compound I sulfate Material C, Compound I tartrate Material A, Compound I tartrate Material B, Compound I tosylate Form I, Compound I tosylate Material A, Compound I tosylate Material C, and Compound I xinafoate Form I as described herein; and one or more pharmaceutically acceptable carriers.

Some embodiments are directed to pharmaceutical compositions comprising a therapeutically effective amount of a compound selected from: a phosphate complex (e.g., a phosphate salt or co-crystal) of Compound I, Compound I phosphate Form I, Compound I phosphate Form II, Compound I phosphate Form III, Compound I phosphate Form IV, Compound I phosphate Form V, and Compound I phosphate (amorphous) as described herein; and one or more pharmaceutically acceptable carriers. In one embodiment, a pharmaceutical composition comprises a therapeutically effective amount of Compound I phosphate Form I; and one or more pharmaceutically acceptable carriers.

Compound I may be administered to a subject orally. For example, Compound I phosphate Form I may be administered to a subject orally. Oral administration may be via, for example, capsule or enteric coated tablets. In making pharmaceutical compositions comprising one or more of the forms of Compound I as described herein, the active ingredient may be diluted by an excipient or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of a capsule, a tablet or pill, or the like.

The compounds disclosed herein (e.g., the forms of Compound I) are useful for treatment of diseases mediated, at least in part, by a bromodomain. Accordingly, the present disclosure provides, in one embodiment, a method for treating a disease mediated, at least in part, by a bromodomain in a patient in need thereof comprising administering a therapeutically effective amount of a form of Compound I as described herein, or a composition or solid dispersion thereof.

In one embodiment, the method for treating a disease mediated, at least in part, by a bromodomain in a patient in need thereof comprises administering a therapeutically effective amount of a phosphate complex of Compound I having a crystalline form as described herein. In one such embodiment, the method comprises administering a phosphate complex of Compound I having a crystalline form as described herein or a composition thereof. In one such embodiment, the method comprises administering a therapeutically effective amount of Compound I phosphate Form I as described herein or a composition thereof.

In one embodiment, the method for treating a disease mediated, at least in part, by a bromodomain in a patient in need thereof comprises administering a therapeutically effective amount of a phosphate complex of Compound I having a substantially amorphous form.

In one embodiment, the method for treating a disease mediated, at least in part, by a bromodomain in a patient in need thereof comprises administering a therapeutically effective amount of a solid dispersion comprising a form of Compound I as described herein. In one such embodiment, the method comprises administering a therapeutically effective amount of a solid dispersion comprising a phosphate complex of Compound I, wherein the phosphate complex of Compound I has a substantially amorphous form.

In one embodiment, the present disclosure provides use of a composition for the treatment of a disease mediated, at least in part, by a bromodomain, wherein the composition comprises a form of Compound I as described herein. In one such embodiment, the composition for such use comprises a phosphate complex of Compound I having a crystalline form as described herein. In one embodiment, the composition for such use comprises Compound I phosphate Form I as described herein.

In one embodiment, the present disclosure provides use of a composition for the treatment of a disease mediated, at least in part, by a bromodomain, wherein the composition comprises a phosphate complex of Compound I having a substantially amorphous form.

In one embodiment, the present disclosure provides use of a composition for the treatment of a disease mediated, at least in part, by a bromodomain, wherein the composition comprises a solid dispersion comprising a form of Compound I as described herein. In one embodiment, the composition for such use comprises a solid dispersion comprising a phosphate complex of Compound I as described herein, wherein the phosphate complex of Compound I has a substantially amorphous form.

In one embodiment, the present disclosure provides use of a composition in the manufacture of a medicament for the treatment of a disease mediated, at least in part, by a bromodomain, wherein the composition comprises a form of Compound I as described herein. In one embodiment, the composition for use in the manufacture of the medicament comprises a phosphate complex of Compound I having a crystalline form as described herein. In one embodiment, the composition for use in the manufacture of the medicament comprises Compound I phosphate Form I as described herein.

In one embodiment, the present disclosure provides use of a composition in the manufacture of a medicament for the treatment of a disease mediated, at least in part, by a bromodomain, wherein the composition comprises a phosphate complex of Compound I as described herein, wherein the phosphate complex has a substantially amorphous form.

In one embodiment, the present disclosure provides use of a composition in the manufacture of a medicament for the treatment of a disease mediated, at least in part, by a bromodomain, wherein the composition comprises a solid dispersion comprising a form of Compound I as described herein. In one embodiment, the composition for use in the manufacture of the medicament comprises a solid dispersion comprising a phosphate complex of Compound I as described herein, wherein the phosphate complex of Compound I has a substantially amorphous form.

In one embodiment, the aforementioned bromodomain is a member of the bromodomain and extraterminal (BET) family. In an exemplary embodiment, the bromodomain is BRD2, BRD3, BRD4, or BRDT.

In one embodiment, the disease is cancer, including hematological cancers, lymphoma, multiple myelomas, leukemia, a neoplasm or a tumor (for example a solid tumor). In one embodiment the disease is a neoplasm or cancer of the colon, rectum, prostate (for example castrate resistant prostate cancer), lung (for example non-small cell lung cancer, and small-cell lung cancer), pancreas, liver, kidney, cervix, uterus, stomach, ovary, breast (for example basal or basal-like breast cancer, and triple-negative breast cancer), skin (for example melanoma), the nervous system (including the brain, meninges, and central nervous system, including a neuroblastoma, a glioblastoma, a meningioma, and a medulloblastoma).

In one embodiment, the disease is a carcinoma. In one embodiment, the disease is hepatocellular carcinoma. In one embodiment the disease is NUT midline carcinoma.

In one embodiment, the disease is a lymphoma. In one embodiment, the disease is a B-cell lymphoma. In one embodiment, the disease is Burkitt's lymphoma. In one embodiment, the disease is diffuse large B-cell lymphoma.

In one embodiment, the disease is multiple myeloma.

In one embodiment, the disease is chronic lymphocytic leukemia.

In one embodiment, the present disclosure provides a method for treating a cancer of the colon in a patient in need thereof comprising administering a therapeutically effective amount of Compound I phosphate Form I as described herein, or a composition or solid dispersion thereof.

In one embodiment, the present disclosure provides a method for treating a cancer of the prostate in a patient in need thereof comprising administering a therapeutically effective amount of Compound I phosphate Form I as described herein, or a composition or solid dispersion thereof.

In one embodiment, the present disclosure provides a method for treating a cancer of the breast in a patient in need thereof comprising administering a therapeutically effective amount of Compound I phosphate Form I as described herein, or a composition or solid dispersion thereof.

In one embodiment, the present disclosure provides a method for treating a lymphoma in a patient in need thereof comprising administering a therapeutically effective amount of Compound I phosphate Form I as described herein, or a composition or solid dispersion thereof.

In one embodiment, the present disclosure provides a method for treating a B-cell lymphoma in a patient in need thereof comprising administering a therapeutically effective amount of Compound I phosphate Form I as described herein, or a composition or solid dispersion thereof.

In one embodiment, the present disclosure provides a method for treating a diffuse large B-cell lymphoma in a patient in need thereof comprising administering a therapeutically effective amount of Compound I phosphate Form I as described herein, or a composition or solid dispersion thereof.

In one embodiment, the present disclosure provides use of a composition for the treatment of a cancer of the colon, wherein the composition comprises Compound I phosphate Form I as described herein, or a solid dispersion thereof.

In one embodiment, the present disclosure provides use of a composition for the treatment of a cancer of the prostate, wherein the composition comprises Compound I phosphate Form I as described herein, or a solid dispersion thereof.

In one embodiment, the present disclosure provides use of a composition for the treatment of a cancer of the breast, wherein the composition comprises Compound I phosphate Form I as described herein, or a solid dispersion thereof.

In one embodiment, the present disclosure provides use of a composition for the treatment of a lymphoma, wherein the composition comprises Compound I phosphate Form I as described herein, or a solid dispersion thereof.

In one embodiment, the present disclosure provides use of a composition in the manufacture of a medicament for the treatment of a B-cell lymphoma, wherein the composition comprises Compound I phosphate Form I as described herein, or a solid dispersion thereof.

In one embodiment, the present disclosure provides use of a composition for the treatment of diffuse large B-cell lymphoma, wherein the composition comprises Compound I phosphate Form I as described herein, or a solid dispersion thereof.

In one embodiment, the present disclosure provides use of a composition in the manufacture of a medicament for the treatment of a cancer of the colon, wherein the composition comprises Compound I phosphate Form I as described herein, or a solid dispersion thereof.

In one embodiment, the present disclosure provides use of a composition in the manufacture of a medicament for the treatment of a cancer of the prostate, wherein the composition comprises Compound I phosphate Form I as described herein, or a solid dispersion thereof.

In one embodiment, the present disclosure provides use of a composition in the manufacture of a medicament for the treatment of a cancer of the breast, wherein the composition comprises Compound I phosphate Form I as described herein, or a solid dispersion thereof.

In one embodiment, the present disclosure provides use of a composition in the manufacture of a medicament for the treatment of a lymphoma, wherein the composition comprises Compound I phosphate Form I as described herein, or a solid dispersion thereof.

In one embodiment, the present disclosure provides use of a composition in the manufacture of a medicament for the treatment of a B-cell lymphoma, wherein the composition comprises Compound I phosphate Form I as described herein, or a solid dispersion thereof.

In one embodiment, the present disclosure provides use of a composition in the manufacture of a medicament for the treatment of diffuse large B-cell lymphoma, wherein the composition comprises Compound I phosphate Form I as described herein, or a solid dispersion thereof.

### Combination Therapy

Patients treated for diseases mediated by BET proteins may benefit from combination drug treatment. For example, a form or forms of Compound I as described herein may be combined with one or more additional therapeutic agents.

In one embodiment, a form of Compound I as described herein may be administered sequentially with the additional therapeutic agent(s). Sequential administration or administered sequentially means that the form of Compound I as described herein and the additional therapeutic agent(s) are administered with a time separation of a few seconds, several minutes, hours, days, or weeks. In one embodiment, the time separation may correspond to about 30 seconds or less, about 15 minutes or less, about 30 minutes or less, about 60 minutes or less, about 1 day, about 2 days, about 3 days, about 4 days, about 5 days, about 6 days, about 7 days, about 2 weeks, about 3 weeks, about 4 weeks, about 5 weeks, about 6 weeks, about 7 weeks, or about 8 weeks. When administered sequentially, the form of Compound I as described herein and the additional therapeutic agent(s) may be administered in two or more administrations, and contained in separate compositions or dosage forms, which may be contained in the same or different package or packages.

In one embodiment, a form of Compound I as described herein may be administered simultaneously with the additional therapeutic agent(s). Simultaneous administration or administered simultaneously means that the form of Compound I as described herein and the additional therapeutic agent(s) are administered with a time separation of no more than a few minutes or seconds, e.g., no more than about 15 minutes, about 10 minutes, about 5 minutes, or 1 minute. When administered simultaneously, the form of Compound I as described herein and the additional therapeutic agent(s) may be in separate compositions or dosage forms, or the same composition or dosage form.

In one embodiment, a form of Compound I as described herein may be combined with one or more additional therapeutic agents in a unitary dosage form (for example for oral administration). In one embodiment, a form of Compound I as described herein may and the one or more additional anti-cancer or anti-inflammatory agents may be separate dosage forms.

The compound described herein may be used or combined with one or more of the additional therapeutic agents. The one or more therapeutic agents include, but are not limited to, an inhibitor, agonist, antagonist, ligand, modulator, stimulator, blocker, activator or suppressor of a gene, ligand, receptor, protein, factor such as the following.

Abelson murine leukemia viral oncogene homolog 1 gene (ABL, such as ABL1), Acetyl-CoA carboxylase (such as ACC1/2), activated CDC kinase (ACK, such as ACK1), Adenosine deaminase, adenosine receptor (such as A2B, A2a, A3), Adenylate cyclase, ADP ribosyl cyclase-1, adrenocorticotropic hormone receptor (ACTH), Aerolysin, AKT1 gene, Alk-5 protein kinase, Alkaline phosphatase, Alpha 1 adrenoceptor, Alpha 2 adrenoceptor, Alpha-ketoglutarate dehydrogenase (KGDH), Aminopeptidase N, AMP activated protein kinase, anaplastic lymphoma kinase (ALK, such as ALK1), Androgen receptor, Angiopoietin (such as ligand-1, ligand-2), Angiotensinogen (AGT) gene, murine thymoma viral oncogene homolog 1 (AKT) protein kinase (such as AKT1, AKT2, AKT3), apolipoprotein A-I (APOA1) gene, Apoptosis inducing factor, apoptosis protein (such as 1, 2), apoptosis signal-regulating kinase (ASK, such as ASK1), Arginase (I), Arginine deiminase, Aromatase, Asteroid homolog 1 (ASTE1) gene, ataxia telangiectasia and Rad 3 related (ATR) serine/threonine protein kinase, Aurora protein kinase (such as 1, 2), Axl tyrosine kinase receptor, Baculoviral IAP repeat containing 5 (BIRC5) gene, Basigin, B-cell lymphoma 2 (BCL2) gene, Bcl2 binding component 3, Bcl2 protein, BCL2L11 gene, BCR (breakpoint cluster region) protein and gene, Beta adrenoceptor, Beta-catenin, B-lymphocyte antigen CD19, B-lymphocyte antigen CD20, B-lymphocyte cell adhesion molecule, B-lymphocyte stimulator ligand, Bone morphogenetic protein-10 ligand, Bone morphogenetic protein-9 ligand modulator, Brachyury protein, Bradykinin receptor, B-Raf proto-oncogene (BRAF), Brc-Abl tyrosine kinase, Bruton's tyrosine kinase (BTK), Calmodulin, calmodulin-dependent protein kinase (CaMK, such as CAMKII), Cancer testis antigen 2, Cancer testis antigen NY-ESO-1, cancer/testis antigen 1B (CTAG1) gene, Cannabinoid receptor (such as CB1, CB2), Carbonic anhydrase, casein kinase (CK, such as CKI, CKII), Caspase (such as caspase-3, caspase-7, Caspase-9), caspase 8 apoptosis-related cysteine peptidase CASP8-FADD-like regulator, Caspase recruitment domain protein-15, Cathepsin G, CCR5 gene, CDK-activating kinase (CAK), Checkpoint kinase (such as CHK1, CHK2), chemokine (C—C motif) receptor (such as CCR2, CCR4, CCR5), chemokine (C—X—C motif) receptor (such as CXCR4, CXCR1 and CXCR2), Chemokine CC21 ligand, Cholecystokinin CCK2 receptor, Chorionic gonadotropin, c-Kit (tyrosine-protein kinase Kit or CD117), Claudin (such as 6, 18), cluster of differentiation (CD) such as CD4, CD27, CD29, CD30, CD33, CD37, CD40, CD40 ligand receptor, CD40 ligand, CD40LG gene, CD44, CD45, CD47, CD49b, CD51, CD52, CD55, CD58, CD66e, CD70 gene, CD74, CD79, CD79b, CD79B gene, CD80, CD95, CD99, CD117, CD122, CDw123, CD134, CDw137, CD158a, CD158b1, CD158b2, CD223, CD276 antigen; clusterin (CLU) gene, Clusterin, c-Met (hepatocyte growth factor receptor (HGFR)), Complement C3, Connective tissue growth factor, COP9 signalosome subunit 5, CSF-1 (colony-stimulating factor 1 receptor), CSF2 gene, CTLA-4 (cytotoxic T-lymphocyte protein 4) receptor, Cyclin Dl, Cyclin G1, cyclin-dependent kinases (CDK, such as CDK1, CDK1B, CDK2-9), cyclooxygenase (such as 1, 2), CYP2B1 gene, Cysteine palmitoyltransferase porcupine, Cytochrome P450 11B2, Cytochrome P450 17, cytochrome P450 17A1, Cytochrome P450 2D6, cytochrome P450 3A4, Cytochrome P450 reductase, cytokine signalling-1, cytokine signalling-3, Cytoplasmic isocitrate dehydrogenase, Cytosine deaminase, cytosine DNA methyltransferase, cytotoxic T-lymphocyte protein-4, DDR2 gene, Delta-like protein ligand (such as 3, 4), Deoxyribonuclease, Dickkopf-1 ligand, dihydrofolate reductase (DHFR), Dihydropyrimidine dehydrogenase, Dipeptidyl peptidase IV, discoidin domain receptor (DDR, such as DDR1), DNA binding protein (such as HU-beta), DNA dependent protein kinase, DNA gyrase, DNA methyltransferase, DNA polymerase (such as alpha), DNA primase, dUTP pyrophosphatase, L-dopachrome tautomerase, echinoderm microtubule like protein 4, EGFR tyrosine kinase receptor, Elastase, Elongation factor 1 alpha 2, Elongation factor 2, Endoglin, Endonuclease, Endoplasmin, Endosialin, Endostatin, endothelin (such as ET-A, ET-B), Enhancer of zeste homolog 2 (EZH2), Ephrin (EPH) tyrosine kinase (such as Epha3, Ephb4), Ephrin B2 ligand, epidermal growth factor, epidermal growth factor receptors (EGFR), epidermal growth factor receptor (EGFR) gene, Epigen, Epithelial cell adhesion molecule (EpCAM), Erb-b2 (v-erb-b2 avian erythroblastic leukemia viral oncogene homolog 2) tyrosine kinase receptor, Erb-b3 tyrosine kinase receptor, Erb-b4 tyrosine kinase receptor, E-selectin, Estradiol 17 beta dehydrogenase, Estrogen receptor (such as alpha, beta), Estrogen related receptor, Eukaryotic translation initiation factor 5A (EIF5A) gene, Exportin 1, Extracellular signal related kinase (such as 1, 2), Extracellular signal-regulated kinases (ERK), Factor (such as Xa, VIIa), farnesoid x receptor (FXR), Fas ligand, Fatty acid synthase, Ferritin, FGF-2 ligand, FGF-5 ligand, fibroblast growth factor (FGF, such as FGF1, FGF2, FGF4), Fibronectin, Fms-related tyrosine kinase 3 (Flt3), focal adhesion kinase (FAK, such as FAK2), folate hydrolase prostate-specific membrane antigen 1 (FOLH1), Folate receptor (such as alpha), Folate, Folate transporter 1, FYN tyrosine kinase, paired basic amino acid cleaving enzyme (FURIN), Beta-glucuronidase, Galactosyltransferase, Galectin-3, Glucocorticoid, glucocorticoid-induced TNFR-related protein GITR receptor, Glutamate carboxypeptidase II, glutaminase, Glutathione S-transferase P, glycogen synthase kinase (GSK, such as 3-beta), Glypican 3 (GPC3), gonadotropin-releaseing hormone (GNRH), Granulocyte macrophage colony stimulating factor (GM-CSF) receptor, Granulocyte-colony stimulating factor (GCSF) ligand, growth factor receptor-bound protein 2 (GRB2), Grp78 (78 kDa glucose-regulated protein) calcium binding protein, molecular chaperone groEL2 gene, Heat shock protein (such as 27, 70, 90 alpha, beta), Heat shock protein gene, Heat stable enterotoxin receptor, Hedgehog protein, Heparanase, Hepatocyte growth factor, HERV-H LTR associating protein 2, Hexose kinase, Histamine H2 receptor, Histone methyltransferase (DOT1L), histone deacetylase (HDAC, such as 1, 2, 3, 6, 10, 11), Histone H1, Histone H3, HLA class I antigen (A-2 alpha), HLA class II antigen, Homeobox protein NANOG, HSPB 1 gene, Human leukocyte antigen (HLA), Human papillomavirus (such as E6, E7) protein, Hyaluronic acid, Hyaluronidase, Hypoxia inducible factor-1 alpha, Imprinted Maternally Expressed Transcript (H19) gene, mitogen-activated protein kinase kinase kinase kinase 1 (MAP4K1), tyrosine-protein kinase HCK, I-Kappa-B kinase (IKK, such as IKKbe), IL-1 alpha, IL-1 beta, IL-12, IL-12 gene, IL-15, IL-17, IL-2 gene, IL-2 receptor alpha subunit, IL-2, IL-3 receptor, IL-4, IL-6, IL-7, IL-8, immunoglobulin (such as G, G1, G2, K, M), Immunoglobulin Fc receptor, Immunoglobulin gamma Fc receptor (such as I, III, IIIA), indoleamine 2,3-dioxygenase (IDO, such as IDO1), indoleamine pyrrole 2,3-dioxygenase 1 inhibitor, insulin receptor, Insulin-like growth factor (such as 1, 2), Integrin alpha-4/beta-1, integrin alpha-4/beta-7, Integrin alpha-5/beta-1, Integrin alpha-V/beta-3, Integrin alpha-V/beta-5, Integrin alpha-V/beta-6, Intercellular adhesion molecule 1 (ICAM-1), interferon (such as alpha, alpha 2, beta, gamma), Interferon inducible protein absent in melanoma 2 (AIM2), interferon type I receptor, Interleukin 1 ligand, Interleukin 13 receptor alpha 2, interleukin 2 ligand, interleukin-1 receptor-associated kinase 4 (IRAK4), Interleukin-2, Interleukin-29 ligand, isocitrate dehydrogenase (such as IDH1, IDH2), Janus kinase (JAK, such as JAK1, JAK2), Jun N terminal kinase, kallikrein-related peptidase 3 (KLK3) gene, Killer cell Ig like receptor, Kinase insert domain receptor (KDR), Kinesin-like protein KIF11, Kirsten rat sarcoma viral oncogene homolog (KRAS) gene, Kisspeptin (KiSS-1) receptor, KIT gene, v-kit Hardy-Zuckerman 4 feline sarcoma viral oncogene homolog (KIT) tyrosine kinase, lactoferrin, Lanosterol-14 demethylase, LDL receptor related protein-1, Leukotriene A4 hydrolase, Listeriolysin, L-Selectin, Luteinizing hormone receptor, Lyase, lymphocyte activation gene 3 protein (LAG-3), Lymphocyte antigen 75, Lymphocyte function antigen-3 receptor, lymphocyte-specific protein tyrosine kinase (LCK), Lymphotactin, Lyn (Lck/Yes novel) tyrosine kinase, lysine demethylases (such as KDM1, KDM2, KDM4, KDM5, KDM6, A/B/C/D), Lysophosphatidate-1 receptor, lysosomal-associated membrane protein family (LAMP) gene, Lysyl oxidase homolog 2, lysyl oxidase protein (LOX), lysyl oxidase-like protein (LOXL, such as LOXL2), Hematopoietic Progenitor Kinase 1 (HPK1), Hepatocyte growth factor receptor (MET) gene, macrophage colony-stimulating factor (MCSF) ligand, Macrophage migration inhibitory fact, MAGEC1 gene, MAGEC2 gene, Maj or vault protein, MAPK-activated protein kinase (such as MK2), Mas-related G-protein coupled receptor, matrix metalloprotease (MMP, such as MMP2, MMP9), Mcl-1 differentiation protein, Mdm2 p53-binding protein, Mdm4 protein, Melan-A (MART-1) melanoma antigen, Melanocyte protein Pmel 17, melanocyte stimulating hormone ligand, melanoma antigen family A3 (MAGEA3) gene, Melanoma associated antigen (such as 1, 2, 3, 6), Membrane copper amine oxidase, Mesothelin, MET tyrosine kinase, Metabotropic glutamate receptor 1, Metalloreductase STEAPI (six transmembrane epithelial antigen of the prostate 1), Metastin, methionine aminopeptidase-2, Methyltransferase, Mitochondrial 3 ketoacyl CoA thiolase, mitogen-activate protein kinase (MAPK), mitogen-activated protein kinase (MEK, such as MEK1, MEK2), mTOR (mechanistic target of rapamycin (serine/threonine kinase), mTOR complex (such as 1,2), mucin (such as 1, 5A, 16), mut T homolog (MTH, such as MTH1), Myc proto-oncogene protein, myeloid cell leukemia 1 (MCL1) gene, myristoylated alanine-rich protein kinase C substrate (MARCKS) protein, NAD ADP ribosyltransferase, natriuretic peptide receptor C, Neural cell adhesion molecule 1, Neurokinin 1 (NK1) receptor, Neurokinin receptor, Neuropilin 2, NF kappa B activating protein, NIMA-related kinase 9 (NEK9), Nitric oxide synthase, NK cell receptor, NK3 receptor, NKG2 A B activating NK receptor, Noradrenaline transporter, Notch (such as Notch-2 receptor, Notch-3 receptor), Nuclear erythroid 2-related factor 2, Nuclear Factor (NF) kappa B, Nucleolin, Nucleophosmin, nucleophosmin-anaplastic lymphoma kinase (NPM-ALK), 2 oxoglutarate dehydrogenase, 2,5-oligoadenylate synthetase, O-methylguanine DNA methyltransferase, Opioid receptor (such as delta), Ornithine decarboxylase, Orotate phosphoribosyltransferase, orphan nuclear hormone receptor NR4A1, Osteocalcin, Osteoclast differentiation factor, Osteopontin, OX-40 (tumor necrosis factor receptor superfamily member 4 TNFRSF4, or CD134) receptor, P3 protein, p38 kinase, p38 MAP kinase, p53 tumor suppressor protein, Parathyroid hormone ligand, peroxisome proliferator-activated receptors (PPAR, such as alpha, delta, gamma), P-Glycoprotein (such as 1), phosphatase and tensin homolog (PTEN), phosphatidylinositol 3-kinase (PI3K), phosphoinositide-3 kinase (PI3K such as alpha, delta, gamma), phosphorylase kinase (PK), PKN3 gene, placenta growth factor, platelet-derived growth factor (PDGF, such as alpha, beta), Platelet-derived growth factor (PDGF, such as alpha, beta), Pleiotropic drug resistance transporter, Plexin B1, PLK1 gene, polo-like kinase (PLK), Polo-like kinase 1, Poly ADP ribose polymerase (PARP, such as PARP1, 2 and 3), Preferentially expressed antigen in melanoma (PRAME) gene, Prenyl-binding protein (PrPB), Probable transcription factor PML, Progesterone receptor, Programmed cell death 1 (PD-1), Programmed cell death ligand 1 inhibitor (PD-L1), Prosaposin (PSAP) gene, Prostanoid receptor (EP4), prostate specific antigen, Prostatic acid phosphatase, proteasome, Protein E7, Protein farnesyltransferase, protein kinase (PK, such as A, B, C), protein tyrosine kinase, Protein tyrosine phosphatase beta, Proto-oncogene serine/threonine-protein kinase (PIM, such as PIM-1, PIM-2, PIM-3), P-Selectin, Purine nucleoside phosphorylase, purinergic receptor P2X ligand gated ion channel 7 (P2X7), Pyruvate dehydrogenase (PDH), Pyruvate dehydrogenase kinase, Pyruvate kinase (PYK), 5-Alpha-reductase, Raf protein kinase (such as 1, B), RAF 1 gene, Ras gene, Ras GTPase, RET gene, Ret tyrosine kinase receptor, retinoblastoma associated protein, retinoic acid receptor (such as gamma), Retinoid X receptor, Rheb (Ras homolog enriched in brain) GTPase, Rho (Ras homolog) associated protein kinase 2, ribonuclease, Ribonucleotide reductase (such as M2 subunit), Ribosomal protein S6 kinase, RNA polymerase (such as I, II), Ron (Recepteur d'Origine Nantais) tyrosine kinase, ROS1 (ROS proto-oncogene 1, receptor tyrosine kinase) gene, Ros1 tyrosine kinase, Runt-related transcription factor 3, Gamma-secretase, S100 calcium binding protein A9, Sarco endoplasmic calcium ATPase, Second mitochondria-derived activator of caspases (SMAC) protein, Secreted frizzled related protein-2, Semaphorin-4D, Serine protease, serine/threonine kinase (STK), serine/threonine-protein kinase (TBK, such as TBK1), signal transduction and transcription (STAT, such as STAT-1, STAT-3, STAT-5), Signaling lymphocytic activation molecule (SLAM) family member 7, six-transmembrane epithelial antigen of the prostate (STEAP) gene, SL cytokine ligand, smoothened (SMO) receptor, Sodium iodide cotransporter, Sodium phosphate cotransporter 2B, Somatostatin receptor (such as 1, 2, 3, 4, 5), Sonic hedgehog protein, Specific protein 1 (Spl) transcription factor, Sphingomyelin synthase, Sphingosine kinase (such as 1, 2), Sphingosine-1-phosphate receptor-1, spleen tyrosine kinase (SYK), SRC gene, Src tyrosine kinase, STAT3 gene, Steroid sulfatase, Stimulator of interferon genes (STING) receptor, stimulator of interferon genes protein, Stromal cell-derived factor 1 ligand, SUMO (small ubiquitin-like modifier), Superoxide dismutase, Survivin protein, Synapsin 3, Syndecan-1, Synuclein alpha, T cell surface glycoprotein CD28, tank-binding kinase (TBK), TATA box-binding protein-associated factor RNA polymerase I subunit B (TAF 1B) gene, T-cell CD3 glycoprotein zeta chain, T-cell differentiation antigen CD6, T-cell immunoglobulin and mucin-domain containing-3 (TIM-3), T-cell surface glycoprotein CD8, Tec protein tyrosine kinase, Tek tyrosine kinase receptor, telomerase, Telomerase reverse transcriptase (TERT) gene, Tenascin, TGF beta 2 ligand, Thrombopoietin receptor, Thymidine kinase, Thymidine phosphorylase, Thymidylate synthase, Thymidylate synthase, Thymosin (such as alpha 1), Thyroid hormone receptor, Thyroid stimulating hormone receptor, Tissue factor, TNF related apoptosis inducing ligand, TNFR1 associated death domain protein, TNF-related apoptosis-inducing ligand (TRAIL) receptor, TNFSF11 gene, TNFSF9 gene, Toll-like receptor (TLR such as 1-13), topoisomerase (such as I, II, III), Transcription factor, Transferase, Transferrin, Transforming growth factor (TGF, such as beta) kinase, Transforming growth factor TGF-β receptor kinase, Transglutaminase, Translocation associated protein, Transmembrane glycoprotein NMB, Trop-2 calcium signal transducer, trophoblast glycoprotein (TPBG) gene, Trophoblast glycoprotein, Tropomyosin receptor kinase (Trk) receptor (such as TrkA, TrkB, TrkC), Tryptophan 5-hydroxylase, Tubulin, Tumor necrosis factor (TNF, such as alpha, beta), Tumor necrosis factor 13C receptor, tumor progression locus 2 (TPL2), Tumor protein 53 (TP53) gene, Tumor suppressor candidate 2 (TUSC2) gene, Tyrosinase, Tyrosine hydroxylase, tyrosine kinase (TK), Tyrosine kinase receptor, Tyrosine kinase with immunoglobulin-like and EGF-like domains (TIE) receptor, Tyrosine protein kinase ABL1 inhibitor, Ubiquitin, Ubiquitin carboxyl hydrolase isozyme L5, Ubiquitin thioesterase-14, Ubiquitin-conjugating enzyme E2I (UBE2I, UBC9), Urease, Urokinase plasminogen activator, Uteroglobin, Vanilloid VR1, Vascular cell adhesion protein 1, vascular endothelial growth factor receptor (VEGFR), V-domain Ig suppressor of T-cell activation (VISTA), VEGF-1 receptor, VEGF-2 receptor, VEGF-3 receptor, VEGF-A, VEGF-B, Vimentin, Vitamin D3 receptor, Proto-oncogene tyrosine-protein kinase Yes, Wee-1 protein kinase, Wilms' tumor antigen 1, Wilms' tumor protein, X-linked inhibitor of apoptosis protein, Zinc finger protein transcription factor or any combination thereof.

As used herein, the term “chemotherapeutic agent” or “chemotherapeutic” (or “chemotherapy” in the case of treatment with a chemotherapeutic agent) is meant to encompass any non-proteinaceous (i.e., non-peptidic) chemical compound useful in the treatment of cancer.

The compound described herein may be used or combined with one or more of the additional therapeutic agents. therapeutic agents may be categorized by their mechanism of action into, for example, the following groups:
anti-metabolites/anti-cancer agents, such as pyrimidine analogs floxuridine, capecitabine, cytarabine, CPX-351 (liposomal cytarabine, daunorubicin), and TAS-118;
purine analogs, folate antagonists (such as pralatrexate), and related inhibitors;
antiproliferative/antimitotic agents including natural products, such as vinca alkaloids (vinblastine, vincristine) and microtubule disruptors such as taxane (paclitaxel, docetaxel), vinblastin, nocodazole, epothilones, vinorelbine (NAVELBINE®), and epipodophyllotoxins (etoposide, teniposide);
DNA damaging agents, such as actinomycin, amsacrine, busulfan, carboplatin, chlorambucil, cisplatin, cyclophosphamide (CYTOXAN®), dactinomycin, daunorubicin, doxorubicin, epirubicin, iphosphamide, melphalan, merchlorethamine, mitomycin C, mitoxantrone, nitrosourea, procarbazine, taxol, Taxotere, teniposide, etoposide, and triethylenethiophosphoramide;
DNA-hypomethylating agents, such as guadecitabine (SGI-110);
antibiotics such as dactinomycin, daunorubicin, doxorubicin, idarubicin, anthracyclines, mitoxantrone, bleomycins, plicamycin (mithramycin);
enzymes such as L-asparaginase which systemically metabolizes L-asparagine and deprives cells which do not have the capacity to synthesize their own asparagine;
antiplatelet agents;
DNAi oligonucleotides targeting Bcl-2, such as PNT2258;
agents that activate or reactivate latent human immunodeficiency virus (HIV), such as panobinostat and romidepsin;
asparaginase stimulators, such as crisantaspase (Erwinase®) and GRASPA (ERY-001, ERY-ASP);
pan-Trk, ROS1 and ALK inhibitors, such as entrectinib;
anaplastic lymphoma kinase (ALK) inhibitors, such as alectinib;
antiproliferative/antimitotic alkylating agents, such as nitrogen mustard cyclophosphamide and analogs (melphalan, chlorambucil, hexamethylmelamine, thiotepa), alkyl nitrosoureas (carmustine) and analogs, streptozocin, and triazenes (dacarbazine);
antiproliferative/antimitotic antimetabolites, such as folic acid analogs (methotrexate);
platinum coordination complexes (cisplatin, oxiloplatinim, and carboplatin), procarbazine, hydroxyurea, mitotane, and aminoglutethimide;
hormones, hormone analogs (estrogen, tamoxifen, goserelin, bicalutamide, and nilutamide), and aromatase inhibitors (letrozole and anastrozole);
anticoagulants such as heparin, synthetic heparin salts, and other inhibitors of thrombin;
fibrinolytic agents such as tissue plasminogen activator, streptokinase, urokinase, aspirin, dipyridamole, ticlopidine, and clopidogrel;
antimigratory agents;
antisecretory agents (breveldin);
immunosuppressives, such as tacrolimus, sirolimus, azathioprine, and mycophenolate;
growth factor inhibitors, and vascular endothelial growth factor inhibitors;
fibroblast growth factor inhibitors, such as FPA14;
angiotensin receptor blockers, nitric oxide donors;
antisense oligonucleotides, such as AEG35156;
DNA interference oligonucleotides, such as PNT2258, AZD-9150;
anti-ANG-2 antibodies, such as MEDI3617, and LY3127804;
anti-MET/EGFR antibodies, such as LY3164530;
anti-EFGR antibodies, such as ABT-414;
anti-CSF1R antibodies, such as emactuzumab, LY3022855, AMG-820;
anti-CD40 antibodies, such as RG7876;
anti-endoglin antibodies, such as TRC105;
anti-CD45 antibodies, such as 131I-BC8 (lomab-B);
anti-HER3 antibodies, such as LJM716;
anti-HER2 antibodies, such as margetuximab, MEDI4276;
anti-HLA-DR antibodies, such as IMMU-114;
anti-IL-3 antibodies, such as JNJ-56022473;
anti-OX40 antibodies, such as MEDI6469, MEDI6383, MEDI0562, MOXR0916, PF-04518600, RG-7888, GSK-3174998;
anti-EphA3 antibodies, such as KB-004;
anti-CD20 antibodies, such as obinutuzumab;
anti-CD20/CD3 antibodies, such as RG7828;
anti-CD37 antibodies, such as AGS67E;
anti-ENPP3 antibodies, such as AGS-16C3F;
anti-FGFR-3 antibodies, such as LY3076226;
anti-folate receptor alpha antibodies, such as IMGN853;
MCL-1 inhibitors, such as AMG-176;
anti-programmed cell death protein 1 (anti-PD-1) antibodies, such as nivolumab (OPDIVO®, BMS-936558, MDX-1106), pembrolizumab (KEYTRUDA®, MK-3477, SCH-900475, lambrolizumab, CAS Reg. No. 1374853-91-4), pidilizumab, BGB-A317, and anti-programmed death-ligand 1 (anti-PD-L1) antibodies such as BMS-936559, atezolizumab (MPDL3280A), durvalumab (MEDI4736), avelumab (MSB0010718C), MEDI0680, and MDX1105-01;
PD-L1/VISTA antagonists such as CA-170;
ATM (ataxia telangiectasia) inhibitors, such as AZD0156;
Bromodomain-containing protein 4 (BRD4) inhibitors, such as birabresib dehydrate, FT-1101, PLX-51107, CPI-0610;
CHK1 inhibitors, such as GDC-0575, LY2606368;
CXCR4 antagonists, such as BL-8040, LY2510924, burixafor (TG-0054), X4P-002;
EXH2 inhibitors, such as GSK2816126;
HER2 inhibitors, such as neratinib, tucatinib (ONT-380);
KDM1 inhibitors, such as ORY-1001, IMG-7289, INCB-59872, GSK-2879552;
CXCR2 antagonists, such as AZD-5069;
GM-CSF antibodies, such as lenzilumab;
Selective estrogen receptor downregulators (SERD), such as fulvestrant (Faslodex®), RG6046, RG6047, and AZD9496;
transforming growth factor-beta (TGF-beta) kinase antagonists, such as galunisertib;
bispecific antibodies, such as MM-141 (IGF-1/ErbB3), MM-111 (Erb2/Erb3), JNJ-64052781 (CD19/CD3);
Mutant selective EGFR inhibitors, such as PF-06747775, EGF816, ASP8273, ACEA-0010, BI-1482694;
Anti-GITR (glucocorticoid-induced tumor necrosis factor receptor-related protein) antibodies, such as MEDI1873;
Adenosine A2A receptor antagonists, such as CPI-444;
Alpha-ketoglutarate dehydrogenase (KGDH) inhibitors, such as CPI-613;
XPO1 inhibitors, such as selinexor (KPT-330);
Isocitrate dehydrogenase 2 (IDH2) inhibitors, such as enasidenib (AG-221);
IDH1 inhibitors such as AG-120, and AG-881 (IDH1 and IDH2);
interleukin-3 receptor (IL-3R) modulators, such as SL-401; Arginine deiminase stimulators, such as pegargiminase (ADI-PEG-20);
antibody-drug conjugates, such as MLN0264 (anti-GCC, guanylyl cyclase C), T-DM1 (trastuzumab emtansine, Kadcycla), milatuzumab-doxorubicin (hCD74-DOX), brentuximab vedotin, DCDT2980S, polatuzumab vedotin, SGN-CD70A, SGN-CD19A, inotuzumab ozogamicin, lorvotuzumab mertansine, SAR3419, isactuzumab govitecan, enfortumab vedotin (ASG-22ME), ASG-15ME;
claudin-18 inhibitors, such as claudiximab;
β-catenin inhibitors, such as CWP-291;
CD73 antagonists, such as MEDI-9447;
c-PIM inhibitors, such as PIM447;
BRAF inhibitors, such as dabrafenib, vemurafenib, encorafenib (LGX818);
sphingosine kinase-2 (SK2) inhibitors, such as Yeliva® (ABC294640);
cell cycle inhibitors, such as selumetinib (MEK1/2), and sapacitabine;
AKT inhibitors such as MK-2206, ipatasertib, afuresertib, and AZD5363;
anti-CTLA-4 (cytotoxic T-lymphocyte protein-4) inhibitors, such as tremelimumab;
c-MET inhibitors, such as AMG-337, savolitinib, tivantinib (ARQ-197), capmatinib, and tepotinib;
Pan-RAF inhibitors, such as LY3009120;
Raf/MEK inhibitors, such as RG7304;
CSF1R/KIT and FLT3 inhibitors, such as pexidartinib (PLX3397);
kinase inhibitors, such as vandetanib;
E selectin antagonists, such as GMI-1271;
differentiation inducers, such as tretinoin;
epidermal growth factor receptor (EGFR) inhibitors, such as osimertinib (AZD-9291);
topoisomerase inhibitors, such as doxorubicin, daunorubicin, dactinomycin, eniposide, epirubicin, etoposide, idarubicin, irinotecan, mitoxantrone, pixantrone, sobuzoxane, topotecan, irinotecan, MM-398 (liposomal irinotecan), vosaroxin and GPX-150;
corticosteroids, such as cortisone, dexamethasone, hydrocortisone, methylprednisolone, prednisone, prednisolone;
growth factor signal transduction kinase inhibitors;
nucleoside analogs, such as DFP-10917;
Axl inhibitors, such as BGB-324;
PARP inhibitors, such as olaparib, rucaparib, veliparib;
Proteasome inhibitors, such as ixazomib, carfilzomib (Kyprolis®);
Glutaminase inhibitors, such as CB-839;
Vaccines, such as peptide vaccine TG-01 (RAS), bacterial vector vaccines such as CRS-207/GVAX, autologous Gp96 vaccine, dendritic cells vaccines, Oncoquest-L vaccine, DPX-Survivac, ProstAtak, DCVAC, ADXS31-142, and rocapuldencel-T (AGS-003), oncolytic vaccine talimogene laherparepvec;
anti-cancer stem cells, such as demcizumab (anti-DLL4, Delta-like ligand 4, Notch pathway), napabucasin (BBI-608);
smoothened (SMO) receptor inhibitors, such as Odomzo® (sonidegib, formerly LDE-225), LEQ506, vismodegib (GDC-0449), BMS-833923, glasdegib (PF-04449913), LY2940680, and itraconazole;
interferon alpha ligand modulators, such as interferon alpha-2b, interferon alpha-2a biosimilar (Biogenomics), ropeginterferon alfa-2b (AOP-2014, P-1101, PEG IFN alpha-2b), Multiferon (Alfanative, Viragen), interferon alpha 1b, Roferon-A (Canferon, Ro-25-3036), interferon alfa-2a follow-on biologic (Biosidus)(Inmutag, Inter 2A), interferon alfa-2b follow-on biologic (Biosidus—Bioferon, Citopheron, Ganapar, Beijing Kawin Technology—Kaferon), Alfaferone, pegylated interferon alpha-1b, peginterferon alfa-2b follow-on biologic (Amega), recombinant human interferon alpha-1b, recombinant human interferon alpha-2a, recombinant human interferon alpha-2b, veltuzumab-IFN alpha 2b conjugate, Dynavax (SD-101), and interferon alfa-nl (Humoferon, SM-10500, Sumiferon);
interferon gamma ligand modulators, such as interferon gamma (OH-6000, Ogamma 100);
IL-6 receptor modulators, such as tocilizumab, siltuximab, AS-101 (CB-06-02, IVX-Q-101);
Telomerase modulators, such as tertomotide (GV-1001, HR-2802, Riavax) and imetelstat (GRN-163, JNJ-63935937);
DNA methyltransferases inhibitors, such as temozolomide (CCRG-81045), decitabine, guadecitabine (S-110, SGI-110), KRX-0402, and azacitidine;
DNA gyrase inhibitors, such as pixantrone and sobuzoxane;
Bcl-2 family protein inhibitors, such as ABT-263, venetoclax (ABT-199), ABT-737, and AT-101;
Notch inhibitors, such as LY3039478, tarextumab (anti-Notch2/3), BMS-906024;
anti-myostatin inhibitors, such as landogrozumab;
hyaluronidase stimulators, such as PEGPH-20;
Wnt pathway inhibitors, such as SM-04755, PRI-724;
gamma-secretase inhibitors, such as PF-03084014;
Grb-2 (growth factor receptor bound protein-2) inhibitors, such as BP1001;
TRAIL pathway-inducing compounds, such as ONC201;
Focal adhesion kinase inhibitors, such as VS-4718, defactinib;
hedgehog inhibitors, such as saridegib, sonidegib (LDE225), glasdegib and vismodegib;
Aurora kinase inhibitors, such as alisertib (MLN-8237), and AZD-2811;
HSPB 1 modulators (heat shock protein 27, HSP27), such as brivudine, apatorsen;
ATR inhibitors, such as AZD6738, and VX-970;
mTOR inhibitors, such as sapanisertib and vistusertib (AZD2014);
Hsp90 inhibitors, such as AUY922, onalespib (AT13387);
Murine double minute (mdm2) oncogene inhibitors, such as DS-3032b, RG7775, AMG-232, and idasanutlin (RG7388);
CD137 agonists, such as urelumab;
Anti-KIR monoclonal antibodies, such as lirilumab (IPH-2102);
Antigen CD19 inhibitors, such as MOR208, MEDI-551, AFM-11, inebilizumab;
CD44 binders, such as A6;
CYP17 inhibitors, such as seviteronel (VT-464), ASN-001, ODM-204;
RXR agonists, such as IRX4204;
hedgehog/smoothened (hh/Smo) antagonists, such as taladegib;
complement C3 modulators, such as Imprime PGG;
IL-15 agonists, such as ALT-803
EZH2 (enhancer of zeste homolog 2) inhibitors, such as tazemetostat, CPI-1205, GSK-2816126;
Oncolytic viruses, such as pelareorep;
DOT1L (histone methyltransferase) inhibitors, such as pinometostat (EPZ-5676);
toxins such as Cholera toxin, ricin, Pseudomonas exotoxin, Bordetella pertussis adenylate cyclase toxin, diphtheria toxin, and caspase activators;
DNA plasmids, such as BC-819
PLK inhibitors of PLK 1, 2, and 3, such as volasertib (PLK1);
WEE1 inhibitors, such as AZD1775;
MET inhibitors, such as merestinib;
Rho kinase (ROCK) inhibitors, such as AT13148;
ERK inhibitors, such as GDC-0994;
IAP inhibitors, such as ASTX660;
RNA polymerase II inhibitors, such has lurbinectedin (PM-1183);
Tubulin inhibitors, such as PM-184;
Toll-like receptor 4 (TL4) agonists, such as G100 and PEPA-10;
Elongation factor 1 alpha 2 inhibitors, such as plitidepsin.
Apoptosis Signal-Regulating Kinase (ASK) Inhibitors: ASK inhibitors include ASK1 inhibitors. Examples of ASK1 inhibitors include, but are not limited to, those described in WO 2011/008709 (Gilead Sciences) and WO 2013/112741 (Gilead Sciences).
Bruton's Tyrosine Kinase (BTK) Inhibitors: Examples of BTK inhibitors include, but are not limited to, (S)-6-amino-9-(1-(but-2-ynoyl)pyrrolidin-3-yl)-7-(4-phenoxyphenyl)-7H-purin-8(9H)-one, acalabrutinib (ACP-196), BGB-3111, HM71224, ibrutinib, M-2951, tirabrutinib (ONO-4059), PRN-1008, spebrutinib (CC-292), TAK-020.
Cluster of Differentiation 47 (CD47) inhibitors: Examples of CD47 inhibitors include, but are not limited to anti-CD47 mAbs (Vx-1004), anti-human CD47 mAbs (CNTO-7108), CC-90002, CC-90002-ST-001, humanized anti-CD47 antibody (Hu5F9-G4), NI-1701, NI-1801, RCT-1938, and TTI-621.
Cyclin-dependent Kinase (CDK) Inhibitors: CDK inhibitors include inhibitors of CDK 1, 2, 3, 4, 6 and 9, such as abemaciclib, alvocidib (HMR-1275, flavopiridol), AT-7519, FLX-925, LEE001, palbociclib, ribociclib, rigosertib, selinexor, UCN-01, and TG-02.
Discoidin Domain Receptor (DDR) Inhibitors: DDR inhibitors include inhibitors of DDR1 and/or DDR2. Examples of DDR inhibitors include, but are not limited to, those disclosed in WO 2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO 2013/034933 (Imperial Innovations).
Histone Deacetylase (HDAC) Inhibitors: Examples of HDAC inhibitors include, but are not limited to, abexinostat, ACY-241, AR-42, BEBT-908, belinostat, CKD-581, CS-055 (HBI-8000), CUDC-907, entinostat, givinostat, mocetinostat, panobinostat, pracinostat, quisinostat (JNJ-26481585), resminostat, ricolinostat, SHP-141, valproic acid (VAL-001), vorinostat.
Indoleamine-pyrrole-2, 3-dioxygenase (IDO1) inhibitors: Examples of IDO1 inhibitors include, but are not limited to, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, and shIDO-ST.
Janus Kinase (JAK) Inhibitors: JAK inhibitors inhibit JAK1, JAK2, and/or JAK3. Examples of JAK inhibitors include, but are not limited to, AT9283, AZD1480, baricitinib, BMS-911543, fedratinib, filgotinib (GLPG0634), gandotinib (LY2784544), INCB039110, lestaurtinib, momelotinib (CYT0387), NS-018, pacritinib (SB 1518), peficitinib (ASP015K), ruxolitinib, tofacitinib (formerly tasocitinib), and XL019.
Lysyl Oxidase-Like Protein (LOXL) Inhibitors: LOXL inhibitors include inhibitors of LOXL1, LOXL2, LOXL3, LOXL4, and/or LOXL5. Examples of LOXL inhibitors include, but are not limited to, the antibodies described in WO 2009/017833 (Arresto Biosciences). Examples of LOXL2 inhibitors include, but are not limited to, the antibodies described in WO 2009/017833 (Arresto Biosciences), WO 2009/035791 (Arresto Biosciences), and WO 2011/097513 (Gilead Biologics).
Matrix Metalloprotease (MMP) Inhibitors: MMP inhibitors include inhibitors of MMP1 through 10. Examples of MMP9 inhibitors include, but are not limited to, marimastat (BB-2516), cipemastat (Ro 32-3555) and those described in WO 2012/027721 (Gilead Biologics).
Mitogen-activated Protein Kinase (MEK) Inhibitors: MEK inhibitors include antroquinonol, binimetinib, cobimetinib (GDC-0973, XL-518), MT-144, selumetinib (AZD6244), sorafenib, trametinib (GSK1120212), uprosertib+trametinib.
Phosphatidylinositol 3-kinase (PI3K) Inhibitors: PI3K inhibitors include inhibitors of PI3Kγ, PI3Kδ, PI3Kβ, PI3Kα, and/or pan-PI3K. Examples of PI3K inhibitors include, but are not limited to, ACP-319, AEZA-129, AMG-319, AS252424, AZD8186, BAY 10824391, BEZ235, buparlisib (BKM120), BYL719 (alpelisib), CH5132799, copanlisib (BAY 80-6946), duvelisib, GDC-0941, GDC-0980, GSK2636771, GSK2269557, idelalisib (Zydelig®), IPI-145, IPI-443, IPI-549, KAR4141, LY294002, LY3023414, MLN1117, OXY111A, PA799, PX-866, RG7604, rigosertib, RP5090, taselisib, TG100115, TGR-1202, TGX221, WX-037, X-339, X-414, XL147 (SAR245408), XL499, XL756, wortmannin, ZSTK474, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences).
Spleen Tyrosine Kinase (SYK) Inhibitors: Examples of SYK inhibitors include, but are not limited to, 6-(1H-indazol-6-yl)-N-(4-morpholinophenyl)imidazo[1,2-a]pyrazin-8-amine, BAY-61-3606, cerdulatinib (PRT-062607), entospletinib, fostamatinib (R788), HMPL-523, NVP-QAB 205 AA, R112, R343, tamatinib (R406), and those described in U.S. Pat. No. 8,450,321 (Gilead Connecticut) and those described in U.S. 2015/0175616.
Toll-like receptor 8 (TLR8) inhibitors: Examples of TLR8 inhibitors include, but are not limited to, E-6887, IMO-4200, IMO-8400, IMO-9200, MCT-465, MEDI-9197, motolimod, resiquimod, VTX-1463, and VTX-763.
Toll-like receptor 9 (TLR9) inhibitors: Examples of TLR9 inhibitors include, but are not limited to, IMO-2055, IMO-2125, lefitolimod, litenimod, MGN-1601, and PUL-042.
Tyrosine-kinase Inhibitors (TKIs): TKIs may target epidermal growth factor receptors (EGFRs) and receptors for fibroblast growth factor (FGF), platelet-derived growth factor (PDGF), and vascular endothelial growth factor (VEGF). Examples of TKIs include, but are not limited to, afatinib, ARQ-087, asp5878, AZD3759, AZD4547, bosutinib, brigatinib, cabozantinib, cediranib, crenolanib, dacomitinib, dasatinib, dovitinib, E-6201, erdafitinib, erlotinib, gefitinib, gilteritinib (ASP-2215), FP-1039, HM61713, icotinib, imatinib, KX2-391 (Src), lapatinib, lestaurtinib, midostaurin, nintedanib, ODM-203, osimertinib (AZD-9291), ponatinib, poziotinib, quizartinib, radotinib, rociletinib, sulfatinib (HMPL-012), sunitinib, and TH-4000.

As used herein, the term “chemotherapeutic agent” or “chemotherapeutic” (or “chemotherapy” in the case of treatment with a chemotherapeutic agent) is meant to encompass any non-proteinaceous (i.e., non-peptidic) chemical compound useful in the treatment of cancer. Examples of chemotherapeutic agents include but not limited to:
alkylating agents such as thiotepa and cyclophosphamide (CYTOXAN®); alkyl sulfonates such as busulfan, improsulfan, and piposulfan; aziridines such as benzodepa, carboquone, meturedepa, and uredepa; ethylenimines and methylamelamines including altretamine, triethylenemelamine, triethylenephosphoramide, triethylenethiophosphoramide, and trimemylolomelamine; acetogenins, especially bullatacin and bullatacinone; a camptothecin, including synthetic analog topotecan; bryostatin, callystatin; CC-1065, including its adozelesin, carzelesin, and bizelesin synthetic analogs; cryptophycins, particularly cryptophycin 1 and cryptophycin 8; dolastatin; duocarmycin, including the synthetic analogs KW-2189 and CBI-TMI; eleutherobin; 5-azacytidine; pancratistatin; a sarcodictyin; spongistatin; nitrogen mustards such as chlorambucil, chlornaphazine, cyclophosphamide, glufosfamide, evofosfamide, bendamustine, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammall and calicheamicin phiI1), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as demopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replinishers such as frolinic acid; radiotherapeutic agents such as Radium-223; trichothecenes, especially T-2 toxin, verracurin A, roridin A, and anguidine; taxoids such as paclitaxel (TAXOL®), abraxane, docetaxel (TAXOTERE®), cabazitaxel, BIND-014; platinum analogs such as cisplatin and carboplatin, NC-6004 nanoplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; trabectedin, triaziquone; 2,2′,2″-tricUorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; NUC-1031; FOLFIRI (fluorouracil, leucovorin, and irinotecan); and pharmaceutically acceptable salts, acids, or derivatives of any of the above.

### Anti-Hormonal Agents

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors.
Examples of anti-estrogens and SERMs include, for example, tamoxifen (including NOLVADEXm), raloxifene, droloxifene, 4-hydroxytamoxifen, trioxifene, keoxifene, LY117018, onapristone, and toremifene (FARESTON®).
Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®).
Examples of anti-androgens include apalutamide, abiraterone, enzalutamide, flutamide, galeterone, nilutamide, bicalutamide, leuprolide, goserelin, ODM-201, APC-100, ODM-204.
Examples of progesterone receptor antagonist include onapristone.

### Anti-Angiogenic Agents

Anti-angiogenic agents include, but are not limited to, retinoid acid and derivatives thereof, 2-methoxyestradiol, ANGIOSTATIN®, ENDOSTATIN®, regorafenib, necuparanib, suramin, squalamine, tissue inhibitor of metalloproteinase-1, tissue inhibitor of metalloproteinase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as 1-azetidine-2-carboxylic acid (LACA), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, metalloproteinase inhibitors such as BB-94, inhibitors of S100A9 such as tasquinimod. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

### Anti-Fibrotic Agents

Anti-fibrotic agents include, but are not limited to, the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. No. 5,021,456, U.S. Pat. No. 5,059,714, U.S. Pat. No. 5,120,764, U.S. Pat. No. 5,182,297, U.S. Pat. No. 5,252,608 relating to 2-(1-naphthyloxymemyl)-3-fluoroallylamine, and US 2004-0248871, which are herein incorporated by reference.
Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.
Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

### Immunotherapeutic Agents

The immunotherapeutic agents include and are not limited to therapeutic antibodies suitable for treating patients. Some examples of therapeutic antibodies include abagovomab, ABP-980, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, CC49, cetuximab, citatuzumab, cixutumumab, clivatuzumab, conatumumab, dacetuzumab, dalotuzumab, daratumumab, detumomab, dinutuximab, drozitumab, duligotumab, dusigitumab, ecromeximab, elotuzumab, emibetuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab (YERVOY®, MDX-010, BMS-734016, and MDX-101), iratumumab, labetuzumab, lexatumumab, lintuzumab, lorvotuzumab, lucatumumab, mapatumumab, matuzumab, milatuzumab, minretumomab, mitumomab, mogamulizumab, moxetumomab, naptumomab, narnatumab, necitumumab, nimotuzumab, nofetumomab, OBI-833, obinutuzumab, ocaratuzumab, ofatumumab, olaratumab, onartuzumab, oportuzumab, oregovomab, panitumumab, parsatuzumab, pasudotox, patritumab, pemtumomab, pertuzumab, pintumomab, pritumumab, racotumomab, radretumab, ramucirumab (Cyramza®), rilotumumab, rituximab, robatumumab, samalizumab, satumomab, sibrotuzumab, siltuximab, solitomab, simtuzumab, tacatuzumab, taplitumomab, tenatumomab, teprotumumab, tigatuzumab, tositumomab, trastuzumab, tucotuzumab, ubilituximab, veltuzumab, vorsetuzumab, votumumab, zalutumumab, and 3F8. Rituximab can be used for treating indolent B-cell cancers, including marginal-zone lymphoma, WM, CLL and small lymphocytic lymphoma. A combination of Rituximab and chemotherapy agents is especially effective.
The exemplified therapeutic antibodies may be further labeled or combined with a radioisotope particle such as indium-111, yttrium-90 (90Y-clivatuzumab), or iodine-131.

In one embodiment, Compound I phosphate Form I may be combined or administered with any of the additional therapeutic agents disclosed herein. In one exemplary embodiment, Compound I phosphate Form I may be combined or administered with enzalutamide.

### Examples

Crystalline forms of Compound I were analyzed by at least one of X-ray powder diffraction (XRPD), differential scanning calorimetry (DSC), thermogravimetric analysis (TGA), dynamic vapor sorption (DVS), solution proton nuclear magnetic resonance spectroscopy (¹H NMR), KF titration, and aqueous vapor stress experiments. XRPD patterns of Compound I were collected with a PANalytical X'Pert PRO MPD diffractometer using mostly the following experimental setting: 45 kV, 40 mA, Kα₁=1.5406 Å, scan range 2-40° 2θ, step size 0.0167° 2θ. The DSC analysis was conducted on a TA Instruments Q2000 differential scanning calorimeter using 10° C./min heating rate over the 20° C. to 250° C. temperature range or above. The TGA analysis was conducted on a TA Instruments 2950 thermogravimetric analyzer using a 10° C./min heating rate over the 20° C. to 350° C. temperature range. ¹H NMR spectra were acquired with an Agilent DD2-400 spectrometer using DMSO-d₆ with tetramethylsilane (TMS). KF analysis was performed using a Mettler Toledo DL39 Karl Fischer titrator with a Stromboli drying oven attachment set at about 110° C. Aqueous vapor stress experiments were conducted by placing a sample in a 85% RH jar for a specified duration.

### 1.1 Forms I and II, Material A, and Amorphous Form of Compound I

1.1.1 Compound I Form I

Compound I Form I is an anhydrous, crystalline form of Compound I, and found to be the most thermodynamically stable form of Compound I.

Compound I Form I was initially obtained by crystallization from the following solvent system (by wt. %): about 2% pyridine, about 2% THF, about 1% water, and about 95% EtOAc. Compound I Form I was also obtained from different solvents and solvent mixtures including acetone/water, heptane/acetone, heptane/DCM, heptane/EtOH, MeCN, BuOAc, DCM, DMF/MTBE, EtOH, IPA, EtOAc, IPAc, MeOH, butanol, MEK, MIBK, 2-MeTHF, NMP/IPE, THF, toluene, and TFE using slurries, evaporation, cooling, lyophilization, and precipitation with anti-solvents.

Compound I Form I can be characterized by an X-ray powder diffractogram comprising the following peaks: 6.4, 8.6, 12.7, 13.9, 17.1, and 22.3° 2θ±0.2° 2θ (FIG. 1). The DSC curve for Compound I Form I shows a single endotherm with onset at about 212° C. (FIG. 2). TGA analysis shows about 1.7% weight loss at about 150-200° C., which may correspond to the loss of residual solvent trapped in the crystal lattice (FIG. 3). KF analysis afforded 0% water. DVS analysis showed that Compound I Form I is slightly hygroscopic with about 0.4% moisture uptake at 90% RH.

1.1.2 Compound I Form II

Compound I Form II is an unstable mono-IPA solvate that may convert to Compound I Form I at ambient conditions. Compound I Form II was obtained by dissolving about 1 g of Compound I in about 15 mL of an IPA/EtOH (5:1) solvent system at about 70° C., followed by slow cooling to room temperature and partial evaporation. The solids were isolated by filtration and dried under vacuum at room temperature.

Compound I Form II was found to be a crystalline material via XRPD analysis. Compound I Form II can be characterized by an X-ray powder diffractogram comprising the following peaks: 10.4, 14.2, 20.0, 21.5, and 26.5° 2θ±0.2° 2θ (FIG. 4). The DSC curve for Compound I Form II shows a small endotherm with onset at about 102° C., and a sharp endotherm with onset at about 213° C. (FIG. 5). TGA analysis shows only about 3.9% of weight loss at about 90 to about 110° C., which is lower than 1 equivalent of IPA indicating that IPA solvate is not stable at ambient conditions and may rapidly convert to Compound I Form I (FIG. 6). 1.1.3 Compound I Material A

Compound I Material A is ap-dioxane solvate that desolvates to Compound I Form I at about 80° C. Compound I Material A was obtained by lyophilizing a solution containing about 113.6 mg of Compound I in about 10 mL of dioxane, and was observed as a mixture with Compound I Form I. The ¹H NMR spectrum of Compound I Material A, present as a mixture with Compound I Form I, is consistent with the structure thereof and shows the presence ofp-dioxane.

Compound I Material A was found to be a crystalline material via XRPD analysis. The X-ray powder diffractogram of the mixture of Compound Form I and Compound I Material A was compared to the diffractogram of Compound I Form I to determine the peaks associated with Compound I Material A (FIG. 7). In particular, the peaks of Compound I Material A were determined by subtraction of the peaks of Compound I Form I from the peaks associated with the mixture of Compound I Form I and Compound I Material A, and include: 8.0, 8.7, 10.2, 10.4, 13.7, 16.1, 17.8, and 22.0° 2θ±0.2° 2θ (FIG. 7). The DSC curve for Compound I Material A, present as a mixture with Compound I Form I, shows an endotherm with onset at about 66° C., followed by melt with onset at about 210° C. (FIG. 8). TGA analysis shows about 3% step weight loss below about 100° C. (FIG. 9). KF analysis afforded minimal water.

1.1.4 Compound I amorphous

Compound I amorphous was obtaining by dissolving about 40.9 mg of Compound I in about 1 mL TFE, following by filtration, evaporation, and about two days of drying at ambient conditions. Compound I amorphous can be characterized by the X-ray powder diffractogram as substantially shown in FIG. 10.

2.1 Salt/Co-Crystal Screen of Compound I

A salt/co-crystal screen was performed using microscale and manual medium scale experiments.

Microscale experiments were carried out using a 96-well plate. The resulting content of the wells was observed under polarized light. Solutions of counterions and co-formers in methanol, methanol/chloroform, tetrahydrofuran, or water (0.1 M) were added to each of the wells of a microplate. Addition of a stock solution of Compound I in dichloromethane (0.1 M) and addition of a third solvent were performed in amounts as to provide a given mol/mol ratio of Compound I to a co-former (about 2-3 mg per well). One well was left blank for reference XRPD. The plate was sonicated for about 26 minutes and left undisturbed to allow for fast evaporation of solvents.

Medium scale experiments were performed on about 40 to about 200 mg scale. Specified co-formers or their aqueous or organic media solutions were combined with solids, solutions, or suspensions of Compound I in various organic solvents at ambient or elevated temperatures. Co-formers were utilized using about 1 to about 3 molar equivalents. Solids, when produced, were typically isolated by vacuum filtration.

Co-formers used in the microscale and/or medium experiments include: benzoic acid, benzenesulfonic acid, caffeine, citric acid, ethanesulfonic acid, ethanedisulfonic acid, fumaric acid, gentisic acid, L-glutamic acid, glycolic acid, hippuric acid, hydrochloride, keto-glutaric acid, L-malic acid, D-mannitol, malonic acid, methanesulfonic acid, nicotinamide, naphthalenesulfonic acid, naphthalenedisulfonic acid, oxalic acid, phosphoric acid, piperazine, L-proline, succinic acid, sulfuric acid, L-tartaric acid, p-toluenesulfonic acid, urea, and xinafoic acid. Exemplary salts were obtained as described below.

2.1.1 Compound I Phosphate Form I

Compound I phosphate Form I is an anhydrous form found to be the most thermodynamically stable form of Compound I phosphate in most solvents.

Compound I phosphate Form I was found to be a crystalline material via XRPD analysis. Compound I phosphate Form I can be characterized by an X-ray powder diffractogram comprising peaks at: 5.0, 12.1, 13.0, 14.9, 15.8, 19.8, 21.7, 23.3, and 27.0° 2θ±0.2° 2θ (FIG. 11). The DSC curve shows a sharp endotherm with onset at about 223° C. (FIG. 12). The TGA analysis shows about 0.4% continuous weight loss below about 150° C. (FIG. 13). The ¹H NMR spectrum of Compound I phosphate Form I is consistent with the structure thereof. KF analysis did not show any significant presence of water. Ion Chromatography analysis confirms about a 1:1 ratio of Compound I/phosphoric acid.

Initially, Compound I phosphate Form I was obtained from combining Compound I Form I and about 1 equivalent of phosphoric acid in a MeOH/IPA (30/70 v/v) solvent mixture. Processes utilizing about 2 or about 3 equivalents of phosphoric acid were also found to yield Compound I phosphate Form I.

In some embodiments, Compound I phosphate Form I, once formed, e.g., via the reactive crystallization methods disclosed herein, may be recrystallized from a variety of single solvents or binary solvent systems (e.g., solvent/anti-solvent systems) to promote desired physical characteristics such as crystal size. Recrystallization of Compound I phosphate Form I may occur with or without the addition of Compound I phosphate Form I seed material. The single solvents and binary solvent systems in which Compound I phosphate Form I may be recrystallized include, but are not limited to, MeOH, MeOH/EtOAc, DMA/MeCN, DMAc/toluene, DMF/MeCN, DMAc/MeCN, DMF/EtOAc, DMF/toluene, DMSO/MeCN, DMSO/MeCN, DMSO/IPA, NMP/MeCN, NMP/IPA, and NMP/EtOAc. A summary of the approximate solubility of Compound I phosphate Form I in various single solvents and binary solvent systems is provided in Tables I and II, respectively.

TABLE 1
Solubility of Compound I Phosphate Form I in Single Solvents

| | Approximate Solubility | | |
|---|---|---|---|
| Solvent | (mg-solid/ml-solvent) | | |
| Name | 20° C. | 50° C. | 80° C. |
| Acetone | 0.48 | | |
| Acetonitrile | 0.23 | | |
| 2-Butanol | remained a slurry | | |
| DCM (form | 1.54 | | |
| changes) | | | |
| DMSO | >92.5 | >185 | >>185 |
| | <185 | | |
| Ethanol | 2.46 | | |
| EtOH/water | 22.7 | | |
| Ethyl acetate | 0.45 | | |

| Hexane | API adhered to vial walls | |
|---|---|---|
| Heptane | <LOD | remained a slurry |

| Isopropyl acetate | 0.37 | | |
|---|---|---|---|
| Methanol | 12.56 | | |
| MTBE | 0.16 | | |
| 2-Propanol/IPA | 1.02 | | |
| 1-Propanol | remained a slurry | | |
| THF | 0.48 | | |
| Toluene | 1.34 | | |
| MEK | 2.14 | | |
| MIBK | 0.31 | | |
| 2-MeTHF | 0.64 | | |
| DMF | >47, <70 | <70 | >70 |
| NMP | >68, <135 | >135 | >>135 |
| DMAc | >52, <78 | >78 | >>78 |
| Acetic Acid | ~190 | >190 | >>190 |
| 1,4-dioxane | remained a slurry | | |
| Water | 12.16 | | |

TABLE 2
Solubility of Compound I Phosphate
Form I in 1:1 (v/v) Binary Solvents

| | Approximate Solubility | | |
|---|---|---|---|
| Solvent | (mg-solid/ml-solvent) | | |
| Name | 20° C. | 50° C. | 80° C. |
| HOAC/Acetone | >21.5, <43 | >43 | >>43 |
| HOAC/MeCN | >30.5 | >>30.5 | >>>30.5 |
| HOAC/EtOAc | >5.2, <13.8 | ~13.8 | >41.5 |
| HOAC/IPA | >15, <22.3 | >22.3, <44.5 | >44.5 |
| HOAC/IPAc | >3.1, <8.3 | ~8.3 | >8.3 |
| HOAC/MIBK | >11, <22 | ~22 | >22 |
| HOAC/2-MeTHF | >3.8, <10.2 | >10.2, <15.3 | ~15.3 |
| DMAc/Acetone | >4.3, <11.3 | ~11.3 | >11.3, <17 |
| DMAc/MeCN | >4.3, <11.5 | ~11.5 | >11.5, <17.3 |
| DMAc/EtOAc | >2.7, <7.2 | >7.2, <10.8 | ~10.8 |
| DMAc/IPA | >5.2, <14 | >14, <21 | ~21 |
| DMAc/IPAc | >2.9, <7.7 | ~7.7 | >8, <11.5 |
| DMAc/MIBK | >5.6, <15 | ~15 | >15, <22.5 |
| DMAc/2-MeTHF | >6.4, 17.2 | ~17.2 | >17.2, <25.8 |
| DMF/Acetone | >7.3, <19.5 | ~19.5 | >19.5, <29.3 |
| DMF/MeCN | >4.1, <11 | ~11 | >11, <16.5 |
| DMF/EtOAc | >4.1, <10.8 | ~10.8 | >10.8, <16.3 |
| DMF/IPA | >5.3, <14 | ~14 | >14, <21 |
| DMF/IPAc | >5.1, <13.7 | ~13.7 | >13.7, <20.5 |
| DMF/MIBK | remained a thin slurry | | ~6 |
| DMF/2-MeTHF | >5.4, <14.3 | ~14.3 | >14.3, <21.5 |
| DMSO/Acetone | >30 | >>30 | >>>30 |
| DMSO/MeCN | >17, <33.5 | >33.5 | >>33.5 |
| DMSO/EtOAc | >26, <51.5 | >51.5 | >>51.5 |
| DMSO/IPA | >14, <21 | >21, <41 | >41 |
| DMSO/IPAc | >32 | >>32 | >>>32 |
| DMSO/MIBK | >25, <50.5 | >50.5 | >>50.5 |
| DMSO/2-MeTHF | >51.5 | >>51.5 | >>>51.5 |
| NMP/MeCN | >3.4, <9 | >9, <14 | >27.5 |
| NMP/Acetone | >12, <24 | >24 | >>24 |
| NMP/EtOAc | >4.5, <12 | >12, <18 | >18, <36 |
| NMP/IPA | >4.1, <11 | >11, <17 | >17, <33 |
| NMP/IPAc | >5.5, <15 | ~15 | >44 |
| NMP/MIBK | >5, <14 | >14, <21 | >21, <41 |
| NMP/2-MeTHF | ~12 | >12, <24 | ~24 |

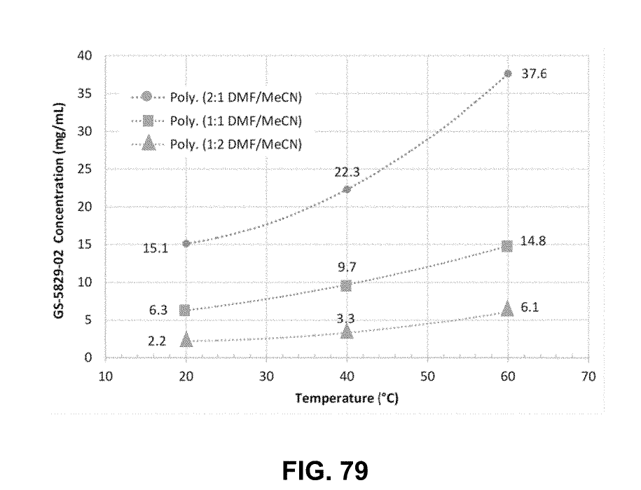

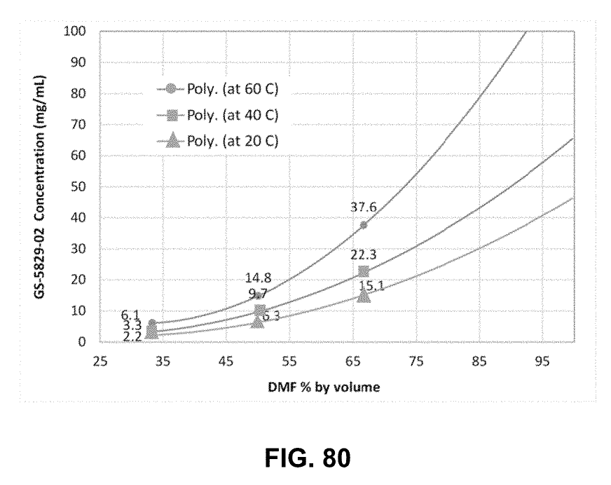

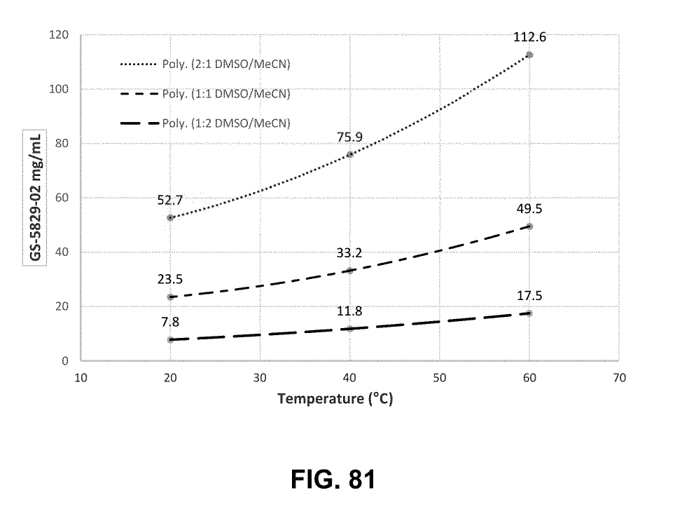

In some embodiments, Compound I phosphate Form I may be recrystallized in DMF/MeOH or DMSO/MeCN solvent systems. The solubility of Compound I phosphate Form I in DMF/MeCN as function of temperature and as a function of the volume percentage of DMF is shown in FIGS. 79 and 80, respectively. The solubility of Compound I phosphate Form I in DMSO/MeCN as function of temperature is shown in FIG. 81.

Exemplary methods for forming and/or recrystallizing Compound I phosphate Form I are provided below.

### Methods 1-3: Reactive Crystallization

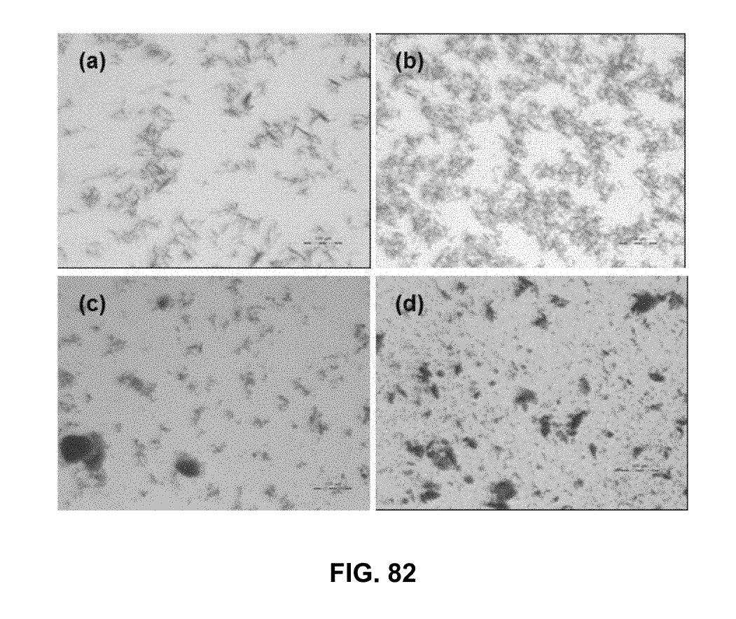

Methods 1, 2, and 3 provide processes directed to the reactive crystallization of Compound I (free base) in MeOH containing solvent systems to form Compound I phosphate Form I. Methods 1, 2, and 3 comprise similar steps; however, in Methods 2 and 3, Compound I phosphate Form I seed crystals (about 10%) are added post complete charge and post 40% charge of H₃PO₄/MeOH, respectively. FIG. 82 provides images taken via polarized light microscopy (PLM) of Compound I phosphate Form I seeds (FIG. 82(a)), and the resulting crystals of Compound I phosphate Form I formed via methods 1-3 (FIG. 82(d)-(b), respectively).

Per method 1, Compound I (1 g) is first dissolved in MeOH (10 g) at about 57° C. A solution of H₃PO₄ (1.06 eq. 0.28 g, 85%) in MeOH (2.5 g) is added thereto at about 55° C. over about 10 to about 20 min. After formation of a slurry, seeds of Compound I Form I (0.5 wt. %, 0.005 g) are added thereto, and the slurry is stirred at about 55° C. for about 6 h. EtOAc (25 g) is added to the slurry over about 4 h at about 55° C. The slurry is agitated at about 55° C. for about 12 h, cooled to about 22° C. over about 2 h, and stirred at about 22° C. for about 1 h. The obtained solids are isolated by vacuum filtration, washed with EtOAc (2 g) then n-heptane (2 g), and dried under vacuum at about 45° C.

Per Method 2, Compound I (1.5 mg) is first dissolved in MeOH (20 mL) at about 57° C. A solution of H₃PO₄ (85%, 0.44 g) in MeOH (2.2 mL) is added thereto at about 57° C. over about 10 to about 20 min. After formation of a slurry, seeds of Compound I phosphate Form I (150 mg) are added thereto, and the resulting slurry is heated to about 60° C. for about 6 h. The slurry is then cooled to: about 58° C. over about 3 h, about 53° C. over about 3 h, about 44° C. over about 2 h, about 31° C. over about 1 h, and about 22° C. over 1 h, followed by aging at about 22° C. for about 2 to about 3 h. The obtained solids are isolated by vacuum filtration, washed with EtOAc then subsequently with n-heptane, and dried under vacuum.

Per Method 3, Compound I (1.5 mg) is first dissolved in MeOH (10 mL) at about 57° C. A solution of H₃PO₄ (85%, 0.44 g) in MeOH (4.56 mL) is formed, and about 2 ml (about 40% of the total) of the H₃PO₄/MeOH solution is added to the Compound I/MeOH solution at about 57° C. over 5-10 min. Seeds of Compound I phosphate Form I (about 60 mg) are next added, and the resulting slurry is held for about 30 min. The remaining H₃PO₄/MeOH solution is added to the slurry at about 57° C. over about 3 h. The slurry is then heated at about 60° C. for about 6 h, followed by cooling to: about 57° C. over about 3 h, about 53° C. over about 3 h, about 44° C. over about 2 h, about 30° C. over about 1 h, and about 22° C. over about 1 h. After aging the slurry at about 22° C. for about 2 to about 3 h, the obtained solids are isolated by vacuum filtration, washed with EtOAc then n-heptane, and dried under vacuum.

### Methods 4-8: Recrystallization in DMF MeOH

Compound I phosphate Form I may be recrystallized in DMF/MeOH via seeding, as described in Methods 4-8 below. Methods 5-8 comprise variations of Method 4 (e.g., with respect to heat-cool cycles, slurry sonication, heat-cool cycling, mother liquor (ML) enrichment, etc.) to promote different crystal characteristics (e.g., crystal size).

Per Method 4, Compound I phosphate Form I seed material is first prepared by slurring Compound I phosphate Form I (0.5 g) in 1:1 DMF/MeCN (16 mL), followed by sonication thereof at about 40° C. for about 3 h. Compound I phosphate Form I (3 g) in DMF (33 mL) is heated to about 65° C., and passed through a medium porosity sintered glass funnel to remove extraneous particles therefrom. MeCN (5.8 mL) is added to the filtrate at about 60° C. to achieve 85:15 v/v DMF/MeCN, and the resulting mixture is seeded with the Compound I phosphate Form I seed material (25 mg, 0.8%). After the resulting mixture forms a slurry, the slurry is cooled to about 20° C. over about 6 h in a parabolic curve (3 cycles). The slurry is then sonicated and subjected to another heating/cooling cycle (about 60° C. to about 20° C. parabolic cooling over about 6 h). After the slurry is heated to about 50° C., MeCN (27.2 mL) is added thereto to achieve 1:1 v/v DMF/MeCN. The slurry is subjected to another 3 cycles of heat-cool between about 60° C. and about 20° C. with parabolic cooling over about 10 h, resulting in crystals.

Method 5 presents a variation of Method 4 in which the batch concentration (1:1 DMF/MeCN slurry of Compound I phosphate Form I) at about 45 mg/mL is readjusted by adding about 2 g of Compound I phosphate Form I. This is achieved by removing a small amount of the mother liquor (ML) and adding about 2 g of Compound I phosphate Form I thereto. The enriched ML is then sonicated for about 1 h before being transferred to the main batch, increasing the batch load from about 45 to about 76 mg/mL (about 60 mL total volume). The batch is then subjected to a heat-cool cycle (temperature: about 70° C. to about 60° C. to about 50° C. to about 40° C. to about 30° C. to about 20° C.; ramp: about 0.1°/min; hold time set at about 3 h).

Method 6 presents another variation of Method 4 in which the DMF ratio is increased to about 55% (concentration about 68 mg/mL), and the slurry is subjected to a heat cool cycle (temperature: about 70° C. to about 60° C. to about 50° C. to about 40° C. to about 30° C. to about 20° C.; ramp: about 0.1°/min; hold time set at about 3 h).

Method 7 presents another variation of Method 4 in which the DMF/MeCN ratio is increased stepwise to about 2:1 by first increasing the DMF ratio to about 60 v % and then to about 67 v %. The slurry is also subjected to a heat cool cycle (temperature: about 70° C. to about 60° C. to about 50° C. to about 40° C. to about 30° C. to about 20° C.; ramp: about 0.1°/min; hold time set at about 3 h). Compound I phosphate Form I recrystallized via Method 7 exhibits uniform crystal growth, where the average particle size thereof is larger than D₉₀˜50 μm.

Method 8 presents yet another variation of Method 4 in which the reaction mixture is allowed to settle, and about 50 mL of the supernatant is withdrawn therefrom. About 1.04 g of Compound I phosphate Form I is added to the supernatant, followed by heating at about 70° C., and re-introduction at 1 mL/min to the batch. The batch is then subjected to three heat-cool cycles, filtered, washed and dried in a vacuum at about 45° C. yielding a uniform crystal size distribution with crystal sizes of about 90 μm x about 20 μm.

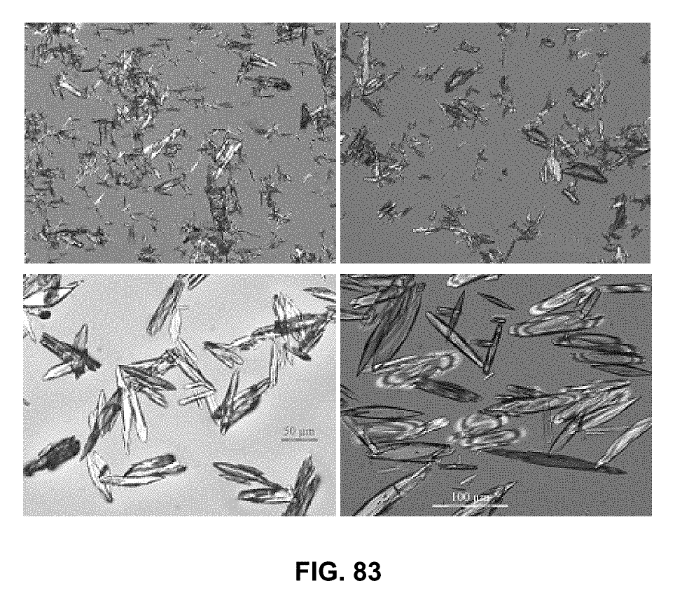

FIG. 83 shows PLM images of Compound I phosphate Form I crystals resulting from recrystallization in different ratios of DMF/MeCN (v/v): (a) 50:50; (b) 55:45; (c) 60:40; and (d) 67:33.

### Method 9: Recrystallization in DMSO/MeCN

Compound I phosphate Form I may be recrystallized in DMSO/MeCN via seeding, as described in Method 9 below.

Per Method 9, Compound I phosphate Form I (4.5 g) is first dissolved in 3:1 DMSO/MeCN (50 mL) at about 55° C. The solution is cooled to about 50° C. and seeded with about 45 mg (1%) of Compound I phosphate Form I seed material. The resulting slurry is cooled from about 50° C. to about 20° C. via a parabolic cooling curve over 20 h, and sonicated at about 40° C. for about 40 min. The slurry is then subjected to three additional heating/cooling cycles (20 h each) of about 55° C. to about 20° C. parabolic cooling, followed by sonication at about 40° C. for about 30 min. After the slurry is heated to about 55° C., MeCN is added thereto to achieve about 1:1 v/v DMSO/MeCN. The slurry is again cooled from about 55° C. to about 20° C. via a parabolic cooling curve over about 20 h.

### Methods 10-12: Crystal Size Optimization

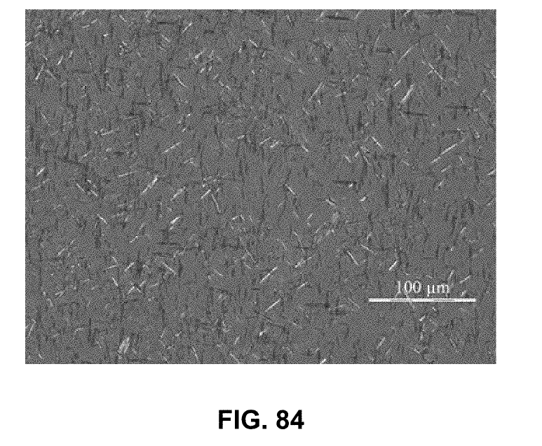

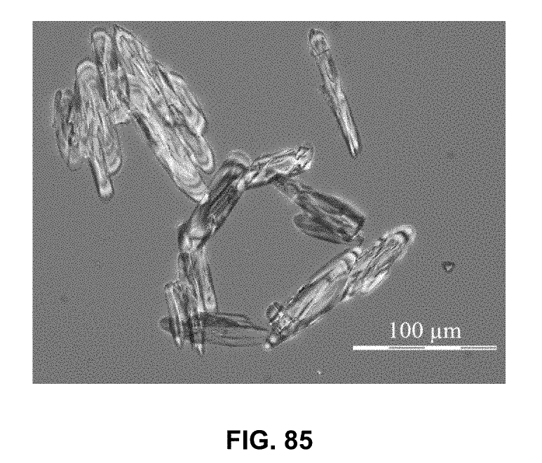

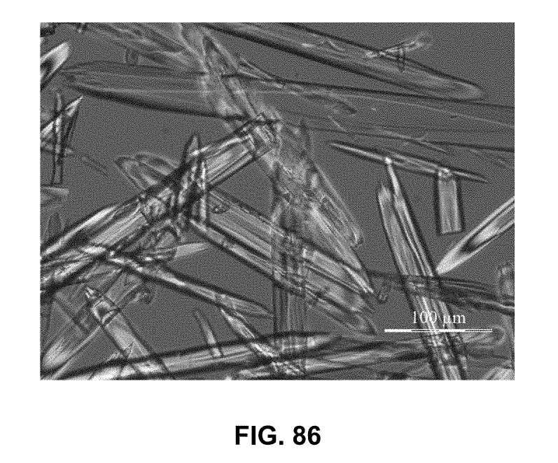

As indicated above, certain experimental conditions during formation and/or recrystallization of Compound I phosphate Form I may be varied to achieve desired physical characteristics thereof. For instance, Method 10 describes an exemplary process in which Compound I phosphate Form I may be formed with a D₉₀ particle size of about 50 μm (FIG. 84). Methods 11 and 12 describe exemplary recrystallization processes in which Compound I phosphate Form may be formed with D₉₀ particle sizes ranging from about 100 μm to about 150 m (FIG. 85), and about 150 μm to about 200 μm, respectively (FIG. 86).

Per Method 10, Compound I (80.0 g) is first dissolved in MeOH (780 mL) at about 50 to about 55° C. H₃PO₄ (85%, 22.4 g) is combined with MeOH (266 mL), and about 42 mL (15% acid) of this acid solution is added to the Compound I/MeOH solution at about 55° C. over 5 to about 10 min. The resulting reaction mixture is seeded with about 1.6 g (2.0%) of Compound I phosphate Form I seed material. After formation of a slurry, the slurry is aged for about 30 min at about 55° C. The remainder of the H₃PO₄/MeOH solution is then added to the slurry over about 8 h at about 55° C., and the resulting slurry mixture is heated at about 60° C. for about 6 h. The slurry is then cooled to about 58° C. over about 6 h, about 53° C. over about 6 h, about 41° C. over about 4 h, about 30° C. over about 2 h, and about 20° C. over about 2 h, followed by aging at about 20° C. for about 2 to about 6 h. The solids are isolated by filtration, washed with EtOAc (500 mL) then heptane (500 mL); and dried.

Per Method 11, Compound I phosphate Form I (about 25 g) is first dissolved in DMF (about 275 mL) at about 60° C. MeCN (about 49 mL) is added thereto at about 60° C., reaching 85:15 DMF/MeCN (v/v). A pre-sonicated seed mixture (2 mL in 1:1 DMSO/MeCN, 0.5 g on dry basis) is also added, and the resulting slurry is aged at about 60° C. for about 1 h. MeCN (88 mL) is added to the slurry at about 60° C. over about 40 h to achieve 2:1 DMF/MeCN (v/v), and the slurry is cooled to: about 58° C. in about 6 h, about 52° C. in about 6 h, about 40° C. in about 5 h, about 30° C. in about 2 h, and about 20° C. in about 1 h, and aged at about 20° C. for about 5 to about 7 h. The slurry is then heated to about 60° C. in about 40 min, followed again by cooling to: about 58° C. in about 5 h, about 52° C. in about 3 h, about 40° C. in about 3 h, about 30° C. in about 2 h, and about 20° C. in about 1 h. After the slurry is aged at about 20° C. for about 2 to about 3 h and allowed to settle, about 285 mL of supernatant is removed and concentrated by removing the majority of the MeCN therefrom. The remaining slurry is sonicated for about 45 min. Compound I phosphate Form I (9 g) is dissolved in the concentrated supernatant at about 70 to about 75° C., followed by addition of MeCN (90 mL) to the supernatant at about 70 to about 75° C. to reach 2:1 DMF/MeCN (v/v). The supernatant comprising Compound I phosphate Form I and 2:1 DMF/MeCN is transferred to the main slurry batch. The batch is heated at about 60° C. for about 3 h, followed by cooling to: about 58° C. in about 6 h, about 52° C. in about 6 h, and about 45° C. in about 5 h. After the batch is sonicated for about 45 min, the batch is again heated at about 60° C. for about 3 h, followed by cooling to: about 58° C. over about 3 h, about 52° C. over about 3 h, about 45° C. over about 2.5 h, about 40° C. over about 2 h, about 30° C. over about 1 h, and about 20° C. over about 1 h, and aged at about 20° C. for about 5 h. About 305 mL of supernatant is subsequently removed and optionally filtered to remove fine solids. Compound I phosphate Form I (4 g) is dissolved in the 305 mL of supernatant at about 65 to about 70° C., and supernatant is added to the main slurry batch at about 60° C. The batch is next aged at about 60° C. for about 4 h, followed by cooling to: about 58° C. in about 6 h, about 52° C. in about 6 h, about 40° C. in about 5 h, about 30° C. in about 2 h, and about 20° C. in about 1 h. About 295 mL of supernatant (2:1 DMF/MeCN v/v, DMF=197 mL, MeCN=98 mL) is then removed, concentrated by removing the majority of the MeCN, diluted with DMF to 197 mL, and subsequently added to the main slurry batch at about 60° C. over about 8 h. MeCN (98 mL) is added separately to the batch over about 12 h. The batch is cooled to about 58° C. in about 3 h, about 52° C. in about 3 h, about 40° C. in about 2.5 h, about 30° C. in about 1 h, and about 20° C. in about 1 h, followed by aging at about 20° C. for about 2 to about 3 h. After the batch is allowed to settle, about 405 mL of supernatant is next removed and put aside (not used for transferring back to the slurry). Compound I Form I (8 g) is dissolved in about 270 mL of DMF at about 65° C., and the resulting solution is filtered and subsequently added to the main slurry batch at about 60° C. over about 9 h. MeCN (135 mL) is added separately to the batch over about 10 h. The batch is next cooled to: about 58° C. in about 3 h, about 52° C. in about 3 h, about 40° C. in about 2.5 h, about 30° C. in about 1 h, and about 20° C. in about 1 h, followed by aging at 20° C. for about 2 to about 3 h. About 300 mL of supernatant is then removed and combined with about 2 g of Compound I phosphate Form I solid at about 65° C., and the resulting solution is added back to the main slurry batch at about 60° C. over about 6.5 h. The batch is cooled to: about 58° C. in about 3 h, about 52° C. in about 3 h, about 40° C. in about 2.5 h, about 30° C. in about 1 h, and about 20° C. in about 1 h, followed by aging at about 20° C. for about 2 to about 3 h. The solids are isolated by filtration, and the wet cake is washed by MeCN 2×100 mL, and dried at RT under vacuum for about 16 h.

Per Method 12, Compound I phosphate Form I (28 g) is first dissolved in DMF (310 mL) at about 60° C. MeCN (55 mL) is added thereto at 60° C., achieving 85:15 DMF/MeCN (v/v). A pre-sonicated seed mixture (2 mL in about 1:1 DMF/MeCN, 0.5 g on dry basis) is also added thereto, and the resulting slurry is aged at about 60° C. for about 30 min. MeCN (100 mL) is added to the slurry at about 60° C. over about 12 h to achieve 2:1 DMF/MeCN (v/v). The slurry is the cooled to: about 58° C. in about 6 h, about 52° C. in about 6 h, about 45° C. in about 5 h, about 40° C. in about 3 h, about 30° C. in about 2 h, and about 20° C. in about 1 h, followed by aging at about 20° C. for about 3 h. After the slurry is allowed to settle, about 250 mL supernatant is removed and combined with about 2 g of Compound I phosphate Form I at about 60 to about 65° C. The supernatant comprising Compound I phosphate Form I is added to the main slurry batch at about 60° C. at about 1 mL/min. The resulting batch is cooled to about 58° C. in about 3 h, about 52° C. in about 3 h, about 45° C. in about 2.5 h, about 40° C. in about 1.5 h, about 30° C. in about 1.5 h, and about 20° C. in about 1 h, followed by aging at about 20° C. for about 2 h. After the slurry is allowed to settle, the majority of the supernatant (about 350 mL) is removed and concentrated by removing the majority of MeCN therefrom. Compound I phosphate Form I (1 g) is dissolved in the concentrated supernatant at about 60 to about 65° C. The concentrated supernatant comprising Compound I phosphate Form I is transferred to the main slurry batch at about 60° C. at about 0.5 mL/min while fresh MeCN (120 mL) is simultaneously added to the batch at about 0.2 mL/min to reach 2:1 DMF/MeCN (v/v). Then batch is then cooled to: about 58° C. in about 3 h, about 52° C. in about 3 h, about 45° C. in about 2.5 h, about 40° C. in about 1.5 h, about 30° C. in about 1.5 h, and about 20° C. in about 1 h, followed by aging at about 20° C. for about 2 h. After the slurry is allowed to settle, the majority of the supernatant (about 400 mL) is again removed and concentrated by removing the majority of MeCN therefrom. Compound I phosphate Form I (1 g) is dissolved in the concentrated supernatant at about 60 to about 65° C. The concentrated supernatant comprising Compound I phosphate Form I is transferred to the main slurry batch at about 60° C. at about 0.5 mL/min while fresh MeCN (120 mL) is simultaneously added to the batch at about 0.2 mL/min reach 2:1 DMF/MeCN (v/v). The batch is next cooled to: about 58° C. in about 3 h, about 52° C. in about 3 h, about 45° C. in about 2.5 h, about 40° C. in about 1.5 h, about 30° C. in about 1.5 h, and about 20° C. in about 1 h, followed by aging at about 20° C. for about 2 h. After the slurry is allowed to settle, the top of the supernatant is next removed (300 mL), heated to about 65° C., and subsequently transferred to the main slurry batch at about 60° C. at about 1.0 mL/min. The batch is subsequently cooled to: about 58° C. in about 3 h, to about 52° C. in 3 h, to about 45° C. in about 2.5 h, to about 40° C. in about 1.5 h, to about 30° C. in about 1.5 h, and to about 20° C. in about 1 h, followed by aging at about 20° C. for about 2 h. The batch is then held at about 60° C. about for 3 h, then cooled to: about 58° C. over about 3 h, about 52° C. over about 3 h, about 45° C. over about 2.5 h, about 40° C. over about 1.5 h, about 30° C. over about 1.5 h, about 20° C. over about 1 h, followed by aging at about 20° C. for about 5 h. The batch is next filtered, washed with about 3×100 mL acetonitrile and vacuum dried at about 45° C. with nitrogen flow.

2.1.2 Compound I Phosphate Form II

Compound I phosphate Form II is an anhydrous form obtained from a slurry comprising about 1 equivalent of phosphoric acid in a MeOH/IPA (1:1) solvent system at about 80° C. The ¹H NMR is spectrum of Compound I phosphate Form II is consistent with the structure thereof, and shows a very small amount of the residual solvent.

Competitive slurries of Compound I phosphate Form I and Compound I phosphate Form II in MeOH/IPA, MeOH/EtOAc or MeOH/IPAc solvent systems afforded Compound I phosphate Form I after overnight slurry at RT, indicating that Compound I Form II is less stable than Compound I phosphate Form I at ambient conditions.

Compound I phosphate Form II was found to be a crystalline material via XRPD analysis. Compound I phosphate Form II can be characterized by an X-ray powder diffractogram comprising peaks at: 5.0, 9.0, 13.4, 14.1, 15.0, 15.3, 19.6, 20.0, and 23.0° 2θ±0.2° 2θ (FIG. 13). The DSC curve shows a sharp endotherm with onset at about 226° C. (FIG. 14). The TGA analysis did not show any weight loss prior to decomposition temperature at about 223° C. (FIG. 14). KF analysis also did not show any presence of water. DVS analysis shows that Compound I phosphate Form II is moderately hygroscopic with about 2.5 to about 3% moisture uptake at about 90% RH.

2.1.3 Compound I Phosphate Form III

Compound I phosphate Form III is a hydrated form, which converts to Compound I phosphate Form I after dehydration at greater than about 150° C. Compound I phosphate Form III was initially obtained from a 1 week water slurry of Compound I phosphate Form I. Compound I Material A was also observed in a hydrate screen of Compound I phosphate Form I in acetone/water at about 0.7 to about 0.95 water activity. Compound I phosphate Form III was further obtained, on a large scale, by slurring about 1 g of Compound I Form I in about 30 mL of water, sonicating the slurry for about 6 min, seeding the sonicated slurry with Compound I phosphate Form III, and stirring at RT overnight.

Compound I phosphate Form III was found to be a crystalline material via XRPD analysis. Compound I phosphate Form III can be characterized by an X-ray powder diffractogram comprising the following peaks at: 5.0, 5.8, 12.7, 14.8, 15.7, 16.1, 17.1, 19.7, 21.9, 22.9, and 24.5° 2θ±0.2° 2θ (FIG. 16). The DSC curve shows a broad endotherm with onset at about 106° C. corresponding to the loss of water, followed by an endotherm with onset at about 212° C. (FIG. 17). The TGA analysis shows about 1.8% step weight loss below about 150° C. (FIG. 18). KF analyses afforded about 1.36% water, which corresponds to about 0.4 equivalent of water. Different lots of Compound I phosphate Form III contained slightly different amount of water by KF analysis (1.22-1.57%), corresponding to about 0.38 to about 0.48 equivalents of water. DVS analysis shows that Compound I phosphate Form III is slightly hygroscopic with about 0.7% moisture uptake at about 90% RH. The solubility of Compound I phosphate Form III in water was found to be about 6 mg/mL.

2.1.4 Compound I Phosphate Form IV

Compound I phosphate Form IV was initially obtained from a slurry of Compound I phosphate Form I in DCM at RT after about 1 week, and was observed as a mixture with Compound I Form phosphate Form I. Compound I phosphate Form IV is an anhydrous form or a desolvated form of the labile DCM solvate. Compound I phosphate Form IV was also obtained, on a large scale, by slurring about 100 mg of Compound I phosphate Form I in about 3 mL of DCM, sonicating the slurry for about 1 minute, and stirring the sonicated slurry at RT for about 5 days. The ¹H NMR spectrum of Compound I phosphate Form IV is consistent with the structure thereof and did not show any residual solvents.

Competitive slurries of Compound I phosphate Form IV and compound I phosphate Form I in a MeOH/EtOAc solvent system showed complete conversion of Compound I phosphate Form IV to Compound I phosphate Form I. Compound I phosphate Form I is more stable than Compound I phosphate Form IV in organic solvents except for DCM most likely due to formation of a labile DCM solvate.

Compound I phosphate Form IV was found to be a crystalline material via XRPD analysis. Compound I phosphate Form IV can be characterized by an X-ray powder diffractogram comprising the following peaks at: 5.0, 9.8, 14.7, 19.7, 26.5, and 29.6° 2θ±0.2° 2θ (FIG. 19). The DSC curve shows an endotherm with onset at about 211° C. (FIG. 20). The TGA analysis shows about 0.4% continuous weight loss below about 150° C., which may correspond to surface water (FIG. 21). KF analysis afforded about 0.53% water.

2.1.5 Compound I Phosphate Form V

Compound I phosphate Form V is a channel solvated/hydrated form obtained from a slurry comprising about 1 equivalent of phosphoric acid in an EtOH/MeOH (12:2 or 10:2) solvent system. The ¹H NMR spectrum of Compound I phosphate Form V is consistent with the structure thereof and shows about 0.36 equivalents of EtOH.

Competitive slurries of Compound I phosphate Form V and Compound phosphate Form I in a MeOH/EtOAc solvent system show complete conversion of Compound I phosphate Form V to Compound I phosphate Form I. Compound I Form IV was found to be less stable than Compound I phosphate Form I.

Compound I phosphate Form V was found to be a crystalline material via XRPD analysis. Compound I phosphate Form V can be characterized by an X-ray powder diffractogram comprising the following peaks at: 5.0, 12.9, 14.0, 14.6, 15.0, 21.6, and 22.0° 2θ±0.2° 2θ (FIG. 22). The DSC curve shows a broad endotherm below about 100° C., and a sharp endotherm with onset at about 222° C. (FIG. 23). The TGA analysis shows about 0.2% weight loss below about 50° C., and about 0.4% weight loss at about 75 to about 160° C. (FIG. 24). KF analysis afforded about 0.78% water.

XRPD analysis of Compound I phosphate Form V after isothermal hold at about 180° C. is consistent with that of Compound I phosphate Form V. The TGA analysis after isothermal hold at about 180° C. shows about 0.4% weight loss below about 150° C. The ¹H NMR spectrum of Compound I phosphate Form V after isothermal hold is also consistent with the structure thereof without any residual solvents.

2.1.6 Compound I Phosphate Amorphous

Compound I phosphate amorphous was prepared by dissolving Compound I phosphate Form I in heptane, and agitating the solution at RT for about several weeks. Compound I phosphate amorphous can be characterized by the X-ray powder diffractogram as substantially shown in FIG. 26, which includes the presence of a small amount of disordered Compound I phosphate material.

2.1.7 Compound I HCl Material A

Compound I HCl Material A was obtained from slurring Compound I (free base) with about 3 equivalents of HCl in acetonitrile, and was observed as a mixture with Compound I HCl Material B.

Compound I HCl Material A was found to be a crystalline material via XRPD analysis. The X-ray powder diffractogram of the mixture of Compound I HCl Material A and Compound I HCl Material B was compared to the diffractogram of Compound I HCl Material B to determine the peaks associated with Compound I HCl Material A (FIG. 25). In particular, the peaks of Compound I HCl Material A were determined by subtraction of the peaks of Compound I HCl Material B from the peaks associated with the mixture of Compound I HCl Material A and Compound I HCl Material B, and include: 11.0, 11.3, 13.5, 17.3, and 19.7° 2θ±0.2° 2θ (FIG. 27).

2.1.8 Compound I HCl Material B

Compound I HCl Material B was obtained from slurring Compound I (free base) with about 3 equivalents of HCl in diethyl ether.

Compound I HCl Material B was found to be a crystalline material via XRPD analysis. Compound I HCl Material B can be characterized by an X-ray powder diffractogram comprising peaks at: 6.7, 9.4, 10.7, 13.8, 16.5, 18.7, 21.4, 21.9, 22.9, 24.8, and 27.0° 2θ±0.2° 2θ (FIG. 28). The DSC curve shows multiple broad endothermic events between about 50° C. and about 250° C., which may corresponds to the loss of volatiles observed by TGA (FIG. 29). The TGA analysis shows about 25% of continuous weight loss up to about 260° C., which may also correspond to the loss of volatiles associated with weakly bound HCl, followed by degradation (FIG. 30). Compound I HCl Material B exhibits a kinetic aqueous solubility of about 6 mg/mL. A high humidity stress test at about 85% RH showed evidence of deliquescence after about 24 h.

2.1.9 Compound I HCl Material C

Compound I HCl Material C was obtained from slurring Compound I (free base) with about 3 equivalents of HCl in isopropanol, and was observed as a mixture with Compound I HCl Material B.

Compound I HCl Material C was found to be a crystalline material via XRPD analysis. The X-ray powder diffractogram of the mixture of Compound I HCl Material C and Compound I HCl Material B was compared to the diffractogram of Compound I HCl Material B to determine the peaks associated with Compound I HCl Material C (FIG. 27). In particular, the peaks of Compound I HCl Material C were determined by subtraction of the peaks of Compound I HCl Material B from the peaks associated with the mixture of Compound I HCl Material C and Compound I HCl Material B, and include: 4.1, 5.4, 8.2, 12.1, 12.3, 12.6, 17.3, 22.6, and 25.4° 2θ±0.2° 2θ.

2.1.10 Compound I HCl Material D

Compound I HCl Material D was obtained from slurries comprising Compound I (free base) and about 3 equivalents of HCl in various solvents or solvent mixtures such as IPA, 1-propanol, MEK, and 2-MeTHF. All isolated solids afforded the same XRPD pattern provided in FIG. 31.

Compound I HCl Material D can be characterized by an X-ray powder diffractogram comprising peaks at: 5.0, 9.0, 13.4, 14.1, 15.0, 15.3, 19.6, 20.0, and 23.0° 2θ±0.2° 2θ (FIG. 31). The DSC curve shows multiple broad endothermic events between about 40° C. and about 250° C., which may correspond to the loss of volatiles. The TGA analysis shows multiple weight losses up to about 260° C., which may also correspond to loss of volatiles associated with residual solvents and weakly bound HCl, followed by degradation.

2.1.11 Compound I HCl Material E

Compound I HCl Material E is a DCM solvate obtained from slurring Compound I (free base) with about 3 equivalents of HCl in DCM, DCM/IPA, or DCM/EtOH solvent systems. Compound I HCl Material E was observed as a mixture with Compound I HCl Material D.

Compound I HCl Material E was found to be a crystalline material via XRPD analysis. The X-ray powder diffractogram of the mixture of Compound I HCl Material E and Compound I HCl Material D was compared to the diffractogram of Compound I HCl Material D to determine the peaks associated with Compound I HCl Material E (FIG. 27). In particular, the peaks of Compound I HCl Material E were determined by subtraction of the peaks of Compound I HCl Material D from the peaks associated with the mixture of Compound I HCl Material E and Compound I HCl Material D, and include: 7.7, 11.3, 12.8, 14.8, 15.4, 16.2, 22.5, and 28.9° 2θ±0.2° 2θ (FIG. 27).

2.1.12 Compound I Sulfate Material A

Compound I sulfate Material A is a hydrated form obtained by volume reduction of a solution comprising Compound I and about 1 equivalent of sulfuric acid in an IPA/MeOH solvent system. The isolated solids afforded the XRPD pattern provided in FIG. 34, which indicates a small amount of Compound I Sulfate Material B. Compound I sulfate Material A was also obtained by vacuum drying solids isolated from a slurry comprising Compound I and about 1 equivalent of sulfuric acid in an IPA at about 75° C. and subsequently at ambient temperatures, or by vacuum drying a sample of Compound I sulfate Material B at about 75° C. The processes utilizing vacuum drying resulted in an XRPD pattern with shifted peaks as compared to the XRPD pattern of FIG. 34.

Compound I sulfate Material A can be characterized by an X-ray powder diffractogram comprising peaks at: 7.3, 10.1, 10.9, 15.5, 16.7, 21.5, 21.9, 22.2, 24.1, and 25.2° 2θ±0.2° 2θ (FIG. 34). The DSC curve shows a broad endotherm with onset at about 70° C. consistent with the loss of volatiles, followed by a sharp endotherm with onset at about 219° C., which may corresponded to melt with decomposition (FIG. 35). The TGA analysis shows about 4.6% weight loss in the about 23 to about 92° C. range, and a gradual weight loss of about 2.2% above about 100° C., which may correspond the beginning of degradation or sublimation (FIG. 36). The ¹H NMR spectrum indicates insufficient amounts of organic solvents present to account for the weight loss in the about 23 to about 92° C. range shown in the TGA analysis, thus this weight loss shown may result from water released during dehydration and correspond to about 1.4 equivalents of water per one mole of the sulfate salt, assuming a 1:1 stoichiometry. TGA analysis of the Compound I sulfate Material A prepared by vacuum drying Compound I sulfate material B shows a smaller weight loss of about 0.3 equivalents of water (about 1%) in the about 23 to about 80° C. range. Compound I sulfate Material A exhibits a kinetic aqueous solubility of about 4 mg/mL, and shows no evidence of deliquescence when stressed about 85% RH for about 24 hours.

2.1.13 Compound I Sulfate Material B

Compound I sulfate Material B is a hydrated form. Compound I sulfate Material B was obtained by suspending about 93 mg Compound I in about 500 μL of IPA, followed by addition of about 1 equivalent of sulfuric acid and 400 μL of IPA. After stirring at room temperature for about 7 days, solids were isolated by filtration. Compound I sulfate Material B converts to sulfate Compound I Material A after vacuum drying at about 75° C. and equilibration at ambient conditions.

Compound I sulfate Material B was found to be a crystalline material via XRPD analysis Compound I sulfate Material B can be characterized by an X-ray powder diffractogram comprising peaks at: 7.1, 10.1, 10.4, 11.6, 14.0, 15.4, 16.0, 17.2, 20.9, 21.1, 22.4, 24.1, 24.3, 24.6, and 27.9° 2θ±0.2° 2θ (FIG. 37). The DSC curve shows a broad endothermic event with onset at about 77° C. consistent with the loss of volatiles, followed by a sharper endotherm with onset at about 214° C. corresponding to melt with decomposition (FIG. 38). The TGA analysis shows about 5.5% weight loss in the about 23 to about 92° C. range due to the loss of volatiles, and a gradual weight loss of about 1.9%, which may correspond to the beginning of degradation or sublimation (FIG. 39). The ¹H NMR spectrum indicates insufficient amount of organic solvents present to account for the weight loss in the about 23 to about 92° C. range, thus this weight loss may result from about 1.7 equivalents of water released during dehydration. Compound I sulfate Material B exhibits a kinetic aqueous solubility of about 4 mg/mL, and shows no evidence of deliquescence when stressed about 85% RH for about 24 hours.

2.1.14 Compound I Sulfate Material C

Compound I sulfate Material C is an IPA solvate obtained by slurring Compound I and 1 equivalent of sulfuric acid in IPA. Compound I sulfate Material C was observed as a mixture with Compound I sulfate Material A. The ¹H NMR spectrum of Compound I sulfate Material C (plus Compound I sulfate Material A) is consistent with the structure and shows significant amount of the residual IPA.

Compound I sulfate Material C was found to be a crystalline material via XRPD analysis. The X-ray powder diffractogram of the mixture of Compound I sulfate Material C and Compound I sulfate material A was compared to the diffractogram of Compound I sulfate Material A to determine the peaks associated with Compound I sulfate Material C (FIG. 40). In particular, the peaks of Compound I sulfate Material C were determined by subtraction of the peaks of Compound I sulfate Material A from the peaks associated with the mixture of Compound I sulfate Material C and Compound I sulfate Material A, and include: 10.7, 13.4, 16.1, 17.2, 18.6, 20.3° 2θ±0.2° 2θ (FIG. 40). Solids obtained after isothermal hold at about 150° C. afforded a crystalline XRPD pattern consistent with Compound I sulfate material A. The DSC curve of Compound I sulfate Material C, present as a mixture with Compound I sulfate Material A, shows a broad endotherm below about 100° C., an endothermic event at about 123° C. which may correspond to a glass transition temperature, and a melting point at about 210° C. (FIG. 41). The TGA analysis shows about 4.4% weight loss below about 100° C. (FIG. 42). KF analysis afforded about 0.68% water.

2.1.15 Compound I Tosylate Form I

Compound I tosylate Form I was obtained by suspending about 59 mg of Compound I in about 500 μL of MEK at room temperature, followed by the addition of about 1 equivalent of p-toluenesulfonic acid, and subsequent stirring at room temperature for about 1 day. The ¹H NMR spectrum of Compound I tosylate Form I is consistent with a 1:1 stoichiometry and shows a very small amount of MEK (about 0.06 equivalents).

Compound I tosylate Form I was found to be a crystalline material via XRPD analysis Compound I tosylate Form I can be characterized by an X-ray powder diffractogram comprising peaks at: 6.2, 6.8, 11.2, 12.4, 13.0, 15.0, 16.7, 18.9, 21.8, 22.7, 23.6, 26.4° 2θ±0.2° 2θ (FIG. 43). The DSC curve shows a very weak broad endotherm below about 100° C. with onset at about 23° C. which may correspond to the loss of residual solvent or moisture, and a sharp endotherm with onset at about 195° C. which may corresponded to melt (FIG. 44). The TGA analysis shows no weight loss prior to about 130° C., followed by a continuous series of weight loss steps which may correspond to degradation or sublimation (FIG. 45). Compound I tosylate Form I exhibits a kinetic aqueous solubility of about 3 mg/mL, and does not show any evidence of deliquescence when stressed at about 85% RH.

2.1.16 Compound I Tosylate Material A

Compound I tosylate Material A was obtained by first suspending about 61 mg of Compound I in about 500 μL of EtOAc at about 60° C., followed by addition of about 1 equivalent ofp-toluenesulfonic acid, and subsequent cooling to afford gummy solids. These gummy solids were isolated and re-slurried in heptane to afford Compound I tosylate Material A and a small amount of amorphous material.

Compound I tosylate Material A was found to be a crystalline material by XRPD analysis. Compound I tosylate Material A can be characterized by an X-ray powder diffractogram comprising peaks at: 5.8, 10.8, 12.1, 13.2, 17.5, 17.8, 19.9, 21.7, 22.6, and 24.4° 2θ±0.2° 2θ (FIG. 46).

2.1.17 Compound I Tosylate Material C

Compound I tosylate Material C was obtained by suspending about 59 mg of Compound I in about 500 μL of EtOAc at room temperature, followed by addition of about 2 equivalents ofp-toluenesulfonic acid and 500 μL of EtOAc, and subsequent stirring at room temperature. Compound I tosylate Material C was observed as a mixture with Compound I tosylate Form I.

Compound I tosylate Material C was found to be a crystalline material via XRPD analysis. With respect to the X-ray powder diffractogram, the peaks of Compound I tosylate Material C were determined by subtraction of the peaks of Compound I tosylate Form I from the peaks associated with the mixture of Compound I tosylate Material C and Compound I tosylate Form I, and include: 6.0, 9.9, 11.7, 12.0, 14.5, 15.4, and 20.9° 2θ±0.2° 2θ (FIG. 47).

2.1.18 Compound I Edisylate Material A

Compound I edisylate Material A was obtained by slurring about 57 mg of Compound I in about 600 μL of isopropanol with about 2 equivalents of ethanedisulfonic acid at room temperature. The ¹H NMR spectrum of compound I edisylate is consistent with Compound I comprising about one equivalent of ethanedisulfonic acid and about 0.1 equivalent of residual isopropanol.

Compound I edisylate material A was found to be a crystalline material via XRPD analysis. Compound I edisylate Material A can be characterized by an X-ray powder diffractogram comprising peaks at: 9.3, 12.4, 15.2, 18.0, 18.9, 19.3, 19.5, 21.3, 22.4, and 24.0° 2θ±0.2° 2θ (FIG. 48). The DSC curve shows a weak broad endotherm below about 100° C. with onset at about 24° C. concurrent with release of volatiles observed by TGA, and a broad endotherm with onset at about 183° C. which may correspond to melting with degradation (FIG. 50). The TGA analysis shows about 0.2% weight loss in the about 25 to about 79° C. range which may correspond to a loss of moisture due to insufficient amounts of organic solvents (FIG. 50). The TGA analysis also shows about 1.3% weight loss between about 100° C. and 197° C., which may correspond to a water release equivalent to about 0.5 moles or degradation (FIG. 50). Compound I edisylate Material A exhibits a kinetic aqueous solubility of about 1 mg/mL, and does not show any signs of deliquescence when stressed at about 85% RH.

2.1.19 Compound I Besylate Material A

Compound I besylate Material A was obtained by first suspending about 62 mg of Compound I in about 500 μL of EtOAc at about 60° C., following by the addition of about 1 equivalent of benzenesulfonic acid, and subsequent cooling to room temperature to afford gummy solids. The gummy solids were isolated and re-slurried in 400 μL of heptane to afford Compound I besylate Material A. The ¹H NMR spectrum of Compound I besylate Material A shows approximately 1 equivalent of benzenesulfonic acid, a very small amount of ethyl acetate (about 0.04 equivalents), and some residual heptane.

Compound I besylate material A was found to be a crystalline material via XRPD analysis. Compound I besylate Material A can be characterized by an X-ray powder diffractogram comprising peaks at: 6.7, 12.5, 12.9, 14.8, 15.2, 17.1, 18.6, 21.0, 21.2, 22.3, 23.6° 2θ±0.2° 2θ (FIG. 51). The DSC curve shows a broad, weak endothermic event below about 100° C. with onset at about 32° C. concurrent with the small initial weight loss on TGA and attributable to the residual solvents loss. The DSC curve also shows a broad exotherm with onset at about 134° C., and a sharp endotherm with onset at about 208° C. which may correspond to melt with decomposition (FIG. 52). The TGA analysis shows about 0.3% weight loss in the about 25-73° C. range which may correspond to a loss of residual organic solvents, and about 1.5% weight loss between about 73° C. and about 182° C. (FIG. 53). Compound I besylate Material A exhibits a kinetic aqueous solubility of about 1 mg/mL, and does not show any signs of deliquescence when stressed at about 85% RH.

2.1.20 Compound I Mesylate Material A

Compound I mesylate Material A was obtained by suspending about 81 mg of Compound I in about 500 μL of 2-MeTHF, followed by the addition of about 1 equivalent of methanesulfonic acid, and stirring at room temperature. Compound I mesylate Material A was observed as a mixture with a small amount of Compound I (free base).

Compound I mesylate material A was found to be a crystalline material via XRPD analysis. Compound I mesylate Material A can be characterized by an X-ray powder diffractogram comprising peaks at: 5.0, 7.3, 7.8, 8.2, 10.0, 11.4, 12.9, 17.9, 21.1, and 21.9° 2θ±0.2° 2θ (FIG. 54). Compound I mesylate Material A exhibits a kinetic aqueous solubility of about 5 mg/mL, and shows evidence of deliquescence at elevated RH.

2.1.21 Compound I Mesylate Material B

Compound I mesylate Material B was obtained by first suspending about 52 mg of Compound I in about 300 μL of toluene, followed by the addition of about 1 equivalent of methanesulfonic acid (as a 0.5 M solution in MeOH) at 65-70° C., and subsequent stirring at room temperature. The mixture was then slowly cooled to room temperature, and the resulting solution was mixed with 200 μL of MTBE and concentrated to dryness by rotary evaporation to afford amorphous solids, which were re-slurried in 600 μL of IPAc at room temperature to afform Compound I mesylate Material B. The ¹H NMR spectrum of Compound I mesylate Material B is consistent with a 1:1 stoichiometry, and shows a small amount of IPAc.

Compound I mesylate material B was found to be a crystalline material via XRPD analysis. Compound I mesylate Material B can be characterized by an X-ray powder diffractogram comprising peaks at: 7.5, 10.6, 11.5, 14.0, 15.3, 18.6, 20.7, 21.0, 23.0, and 24.3° 2θ±0.2° 2θ (FIG. 55). The DSC curve shows a small endotherm with onset at about 186° C., and a sharp endotherm with onset at about 229° C. corresponding to melt with decomposition (FIG. 56). The TGA analysis shows no weight loss prior to about 130° C., and about 1.2 wt. % loss between about 132° C. and about 206° C. (FIG. 57).

2.1.22 Compound I Mesylate Material C

Compound I mesylate Material C is a monohydrate obtained by suspending about 100 mg of Compound I in about 1.1 mL of 2-MeTHF, followed by the addition of about 3 equivalents of methanesulfonic acid, and subsequent stirring at room temperature and drying isolated solids at ambient conditions. The ¹H NMR spectrum indicates that Compound I mesylate Material C has 1:3 ratio of Compound I to the counterion, and no residual organic solvents.

Compound I mesylate material C was found to be a crystalline material via XRPD analysis. Compound I mesylate Material C can be characterized by an X-ray powder diffractogram comprising peaks at: 5.0, 9.3, 10.0, 10.2, 13.5, 15.0, 17.1, 18.2, 20.8, 21.2, 21.8, 22.3, 23.6, 25.8, and 29.4° 2θ±0.2° 2θ (FIG. 58). The DSC curve shows several broad endotherms with onsets at about 35° C., about 96° C., and about 139° C., which may correspond to volatilization followed by decomposition (FIG. 59). The TGA analysis shows about 4.3% weight loss below about 110° C. corresponding to about 1 mol of water (FIG. 60). Compound I mesylate Material C exhibits a kinetic aqueous solubility of about 13 mg/mL, and a tendency to deliquesce at elevated relative humidity.

2.1.23 Compound I Mesylate Material D

Compound I mesylate Material D was obtained by suspending about 84 mg of Compound I in about 1 mL of IPAc, followed by the addition of about 1 equivalent of methanesulfonic acid, and subsequent stirring at room temperature. Compound I mesylate Material D was obtained as a mixture with Compound I mesylate Material B.

Compound I mesylate material D was found to be a crystalline material via XRPD analysis. With respect to the X-ray powder diffractogram, the peaks of Compound I mesylate Material D were determined by subtraction of the peaks of Compound I mesylate Material B from the peaks associated with the mixture of Compound I mesylate Material D and Compound I mesylate Material B, and include 9.8, 11.8, 12.9, 16.8, 17.5, 18.8, and 22.0° 2θ±0.2° 2θ (FIG. 61).

2.1.24 Compound I Mesylate Material E

Compound I mesylate Material E was obtained by combining about 99 mg of Compound I in about 1.5 mL of 2-MeTHF with about 2 equivalents of methanesulfonic acid. This process additionally afforded an amorphous material.

Compound I mesylate material E was found to be a crystalline material via XRPD analysis. Compound I mesylate Material E can be characterized by an X-ray powder diffractogram comprising peaks at: 6.9, 8.7, 9.3, 10.0, 11.7, 13.0, 17.5, 20.7, 20.9, and 23.4° 2θ±0.2° 2θ (FIG. 62).

2.1.25 Compound I Mesylate Material F

Compound I mesylate Material F was obtained by combining Compound I and about 1 equivalent of methanesulfonic acid in IPAc.

Compound I mesylate material F was found to be a crystalline material via XRPD analysis. Compound I mesylate Material F can be characterized by an X-ray powder diffractogram comprising peaks at: 5.1, 7.8, 8.3, 10.0, 13.1, 18.1, 20.0, 22.0, 22.6, 24.1, and 27.5° 2θ±0.2° 2θ (FIG. 63).

2.1.26 Compound I Mesylate Material G

Compound I mesylate Material G was obtained by combining Compound I and about 1 equivalent of methanesulfonic acid in IPAc, and vacuum drying the resulting mixture at about 40° C. The ¹H NMR spectrum of Compound I mesylate Material G was consistent with the structure thereof, and shows about 1 equivalent of methanesulfonic acid and about 0.1 equivalent of residual IPAc.

Compound I mesylate Material G was found to have a slightly disordered XRPD pattern with amorphous content. Compound I mesylate Material G can be characterized by an X-ray powder diffractogram comprising peaks at: 5.1, 5.5, 6.5, 7.9, 10.2, 11.0, 14.9, 17.7, 19.6, 22.4, 24.6° 2θ±0.2° 2θ (FIG. 64). The DSC curve shows a broad endotherm below 80° C., and a broad endotherm with onset at about 142° C. (FIG. 65). The TGA analysis shows about 1.1% weight loss below about 80° C., and about 1.0% weight loss at about 80 to about 140° C. (FIG. 66). DVS analysis shows that Compound I mesylate Material G is very hygroscopic with about 25% moisture uptake at about 90% RH.

2.1.27 Compound I Napsylate Material A

Compound I napsylate Material A was obtained by slurring about 81 mg of Compound I in 2-MeTHF at ambient temperature with about 1 equivalent of naphthalenesulfonic acid.

Compound I napsylate Material A was found to be a crystalline material via XRPD analysis. Compound I napsylate Material A can be characterized by an X-ray powder diffractogram comprising peaks at: 5.5, 10.6, 11.3, 11.9, 15.1, 16.7, 19.4, 22.0, 22.8, and 25.0° 2θ±0.2° 2θ (FIG. 67). Compound I napsylate Material A exhibits a kinetic aqueous solubility below about 1 mg/mL, and does not deliquesce when stressed at 85% RH for about 24 hours.

2.1.28 Compound I Tartrate Material A

Compound I tartrate Material A was obtained by suspending Compound I in about 500 μL EtOAc, followed by the addition of about 2 equivalents of L-tartaric acid at about 55-60° C. and subsequently cooling and stirring at room temperature. Resulting solids were isolated affording a mixture of Compound I tartrate Material A and free L-tartaric acid. The solids were then resuspended in about 300 μL IPA, and stirred at room temperature to provide Compound I tartrate Material A. The ¹H NMR spectrum of Compound I tartrate Material A is consistent with approximately 1:1 stoichiometry, and shows about 0.05 equivalent of residual ethyl acetate.

Compound I tartrate material A was found to be a crystalline material via XRPD analysis. Compound I tartrate Material A can be characterized by an X-ray powder diffractogram comprising peaks at: 10.7, 11.7, 13.9, 17.8, 19.8, 20.5, 22.2, 22.8, and 25.4° 2θ±0.2° 2θ (FIG. 68). Compound I tartrate Material A exhibits a kinetic aqueous solubility of about 15 mg/mL, and does not deliquesce when stressed at about 85% RH.

2.1.29 Compound I Tartrate Material B

Compound I tartrate Material B is an IPA solvate. Compound I Material B was obtained by first slurring about 100 mg of Compound I in about 2 mL of IPA at about 55° C., followed by the addition of about 1.1 equivalent of L-tartaric acid (38 mg). The reaction mixture was stirred at about 55° C. for about 4 hours, followed by cooling to room temperature, and stirring at room temperature overnight. The solids were isolated by vacuum filtration, washed with IPA, and dried under vacuum at about 40° C. The ¹H NMR spectrum of Compound I tartrate Material B was consistent with the structure thereof, and shows about 1 equivalent of tartaric acid and about 0.58 equivalent of IPA.

Compound I tartrate material B was found to be a crystalline material via XRPD analysis. Compound I tartrate Material B can be characterized by an X-ray powder diffractogram comprising peaks at: 5.0, 11.5, 12.7, 14.9, 15.2, 17.4, 20.8, and 21.3° 2θ±0.2° 2θ (FIG. 69). The DSC curve shows multiple endotherms including: a broad endotherm below about 60° C., a sharp endotherm with onset at about 133° C., and an endotherm with onset at about 160° C. (FIG. 70). The TGA analysis shows about 1.3% step weight loss at about 100 to about 150° C. (FIG. 71). KF analysis afforded about 0.64% water.

XRPD analyses were conducted for samples of Compound I tartrate Material B after isothermal holds at about 110° C. and about 150° C., showing no form change after isothermal hold at about 110° C. and mostly amorphous material after isothermal hold at about 150° C. The ¹H NMR spectrum of Compound I tartrate Material B after isothermal hold at about 110° C. was consistent with the structure thereof, and shows about 1 equivalent of tartaric acid and about 0.38 equivalents of IPA. The ¹H NMR spectrum of Compound I tartrate Material B after isothermal hold at about 150° C. was also consistent with the structure thereof, and shows about 1 equivalent of tartaric acid and no residual IPA. These data confirms that Compound I tartrate Material B is a variable IPA solvate.

2.1.30 Compound I Xinafoate Form I

Compound I xinafoate Form I is an anhydrous form. Compound I xinafoate Form I was obtained by slurring Compound I with about 1 equivalent of xinafoic acid in ethyl acetate at ambient temperature. Compound I xinafoate Form I was also obtained by slurring Compound I with about 1 equivalent of xinafoic acid in an ethyl acetate/methanol solution, and cooling the slurry to sub-ambient temperatures. The ¹H NMR spectrum is consistent with Compound I containing xinafoate counterion, and comprises peaks attributable to residual ethyl acetate (about 0.1 equivalent).

Compound I xinafoate Form I was found to be a crystalline material via XRPD analysis. Compound I xinafoate Form I can be characterized by an X-ray powder diffractogram comprising peaks at: 5.5, 11.5, 12.5, 15.3, 16.0, 16.5, 18.3, 20.1, 21.0, 22.5, and 22.9° 2θ±0.2° 2θ (FIG. 72). The DSC curve shows a sharp endotherm at about 184° C. (calculated onset) immediately followed by a sharp exotherm, typical of melt simultaneous with degradation (FIG. 73). The TGA analysis shows no weight loss prior to about 174° C., which is indicative of its unsolvated (anhydrous) nature (FIG. 74). Compound I xinafoate Form I exhibits a kinetic aqueous solubility below 1 mg/mL, and does not deliquesce when stressed at about 85% RH.

2.1.31 Compound I Gentisate Material A

Compound I gentisate Material A was obtained by slurring about 52 mg of Compound I in about 1 mL of EtOAc with about 1 equivalent of gentisic acid at ambient temperature followed by vacuum during solids isolated from the slurry. The ¹H NMR spectrum for Compound I gentisate Material A is consistent with approximately 1:1 stoichiometry, and comprises peaks attributable to residual ethyl acetate (about 0.04 equivalents).

Compound I gentisate Material A was found to be a crystalline material via XRPD analysis. Compound I gentisate Material A can be characterized by an X-ray powder diffractogram comprising peaks at: 6.5, 7.1, 12.6, 13.0, 13.3, 13.6, 15.9, 17.5, 19.5, 22.2, 23.9, and 25.4° 2θ±0.2° 2θ (FIG. 75). The DSC curve shows a small endotherm with onset at about 189° C. The DSC curve also shows a sharp endotherm with onset at about 213° C., immediately followed by a sharp exotherm, which may correspond to a melt followed by degradation (FIG. 76). The TGA thermogram shows no weight loss below about 100° C. The TGA thermogram additionally shows a weight loss of about 0.8% from about 100° C. to about 190° C. (FIG. 77). Compound I gentisate Material A exhibits a kinetic aqueous solubility below about 1 mg/mL, and does not deliquesce when stressed at 85% RH.

2.1.32 Compound I Oxalate (Disordered)

Compound I oxalate is a disordered material obtained by contacting about 50 mg of Compound I with about 700 μL of IPA and about 1 equivalent of oxalic acid at 65-70° C., cooling the mixture to about 55° C., and a subsequent isolation. Compound I oxalate (disordered) can be characterized by an X-ray powder diffractogram comprising peaks at: 5.6, 8.4, 11.9, 14.3, 17.2, 19.7, 21.7, and 22.5° 2θ±0.2° 2θ (FIG. 78). The ¹H NMR spectrum of Compound I oxalate is consistent with the structure thereof.

### 3.1 Stability and Dissolution

3.1.1 Compound I

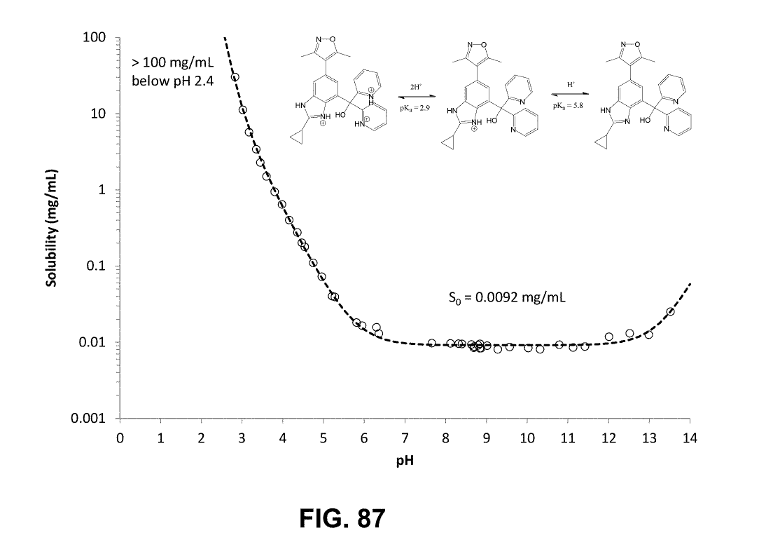

Compound I has multiple ionization constants with weak base pKa values of 2.9, 2.9 and 5.8 (FIG. 85). The aqueous solubility profile of Compound I is pH dependent, with an intrinsic solubility of 9.2 μg/mL observed between about pH 8 and about 11, and a solubility greater than 100 mg/mL observed below about pH 2.4 (FIG. 87).

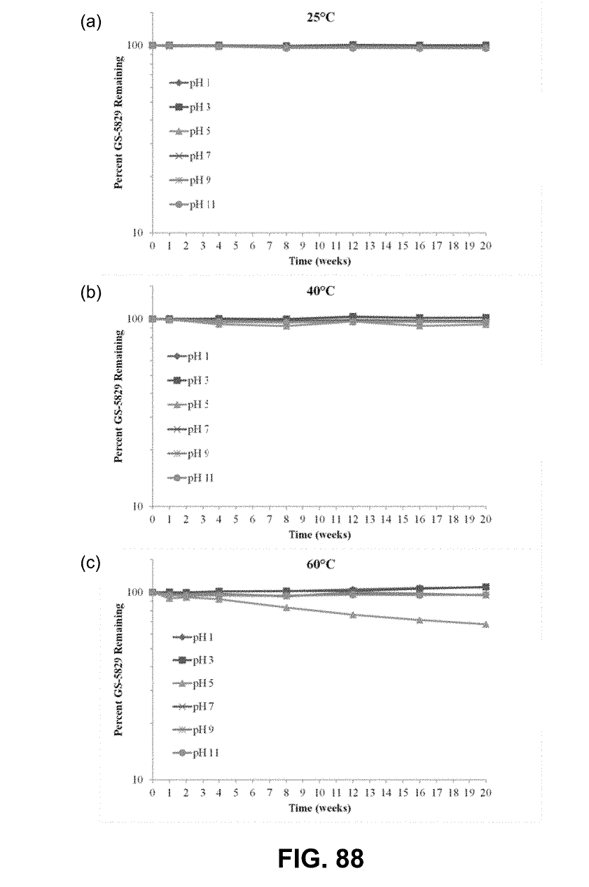

Analysis of the chemical stability of Compound I in aqueous solutions as a function of pH and temperature shows that Compound I is not susceptible to acid- or base-catalyzed degradation (FIG. 88). In particular, no significant degradation of Compound I between about pH 1 and about pH 11 is observed at about 25° C. (FIG. 88(a)), or about 40° C. (FIG. 88(b)) over a period of about 20 weeks. Additionally, no significant degradation was observed at about 60° C. over the same pH range (FIG. 88(c)) with the exception of samples stored at pH 5, where formation of small amount of an aluminum complex with a Compound I to aluminum molar ratio of 3:1 was observed by LC/MS analysis.

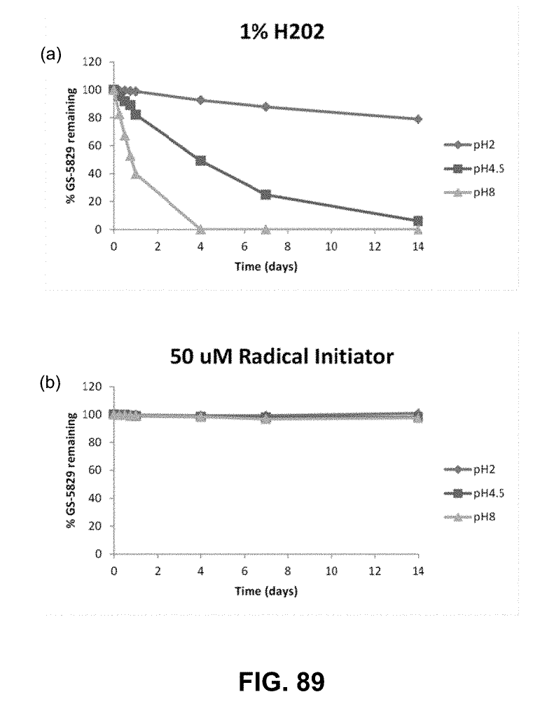

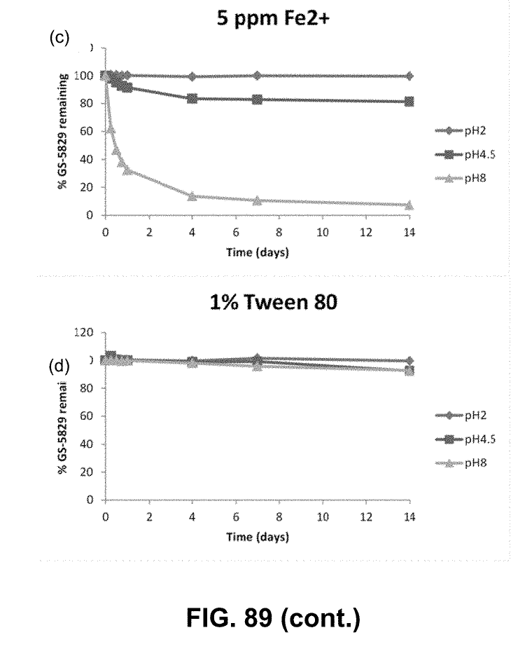

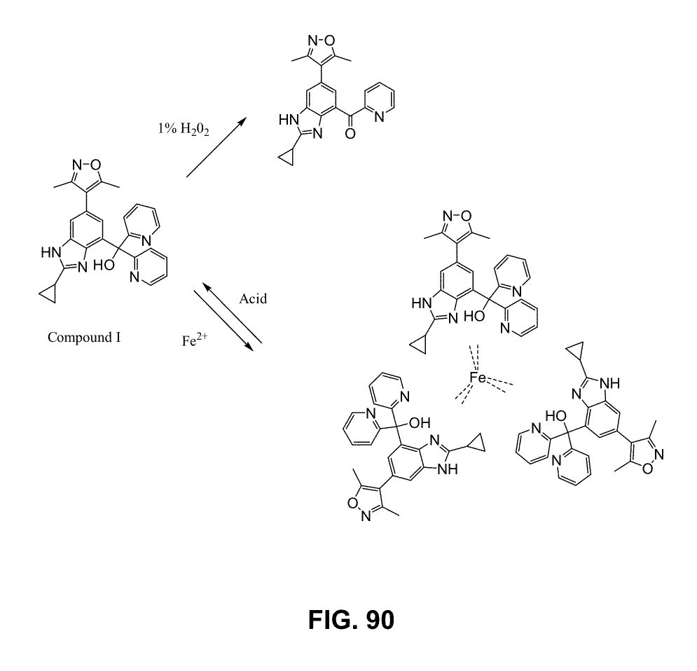

FIG. 89 shows the susceptibility of Compound I to oxidation in solution as a function of pH in solutions kept at about 40° C. and which comprise hydrogen peroxide (FIG. 89(a)), radical initiator (FIG. 89(b)), iron (II) ions (FIG. 89(c)), and polysorbate 80 (FIG. 89(d)). No significant degradation was observed in solutions containing radical initiator or polysorbate 80 over a period of about 14 days, as shown in FIGS. 89(b) and (d), respectively. However, solutions containing hydrogen peroxide or iron (II) ions did show loss of Compound I over the same time period with increased degradation at higher pH values, as shown in FIGS. 89(a) and (b), respectively. Analysis of these solutions by LC/MS showed the primary product formed in the presence of hydrogen peroxide may be due to loss of a single pyridine group (FIG. 90). In the presence of iron (II) ions, formation of a complex with iron is observed at high pH (FIG. 90). This complex can be reversed to yield Compound I by addition of a strong acid.

3.1.2 Compound I Phosphate Form I

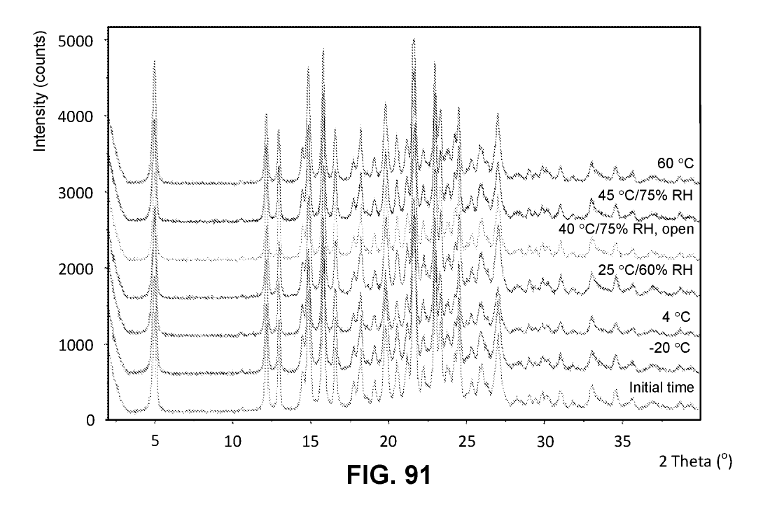

Compound I phosphate Form I is chemically stable at various conditions for about 1 month, as shown by the XRPD patterns of FIG. 91, and the stability data provided in Table 3.

TABLE 3
Chemical Stability of Compound I
Phosphate Form I After 1 Month

| Condition | % AN | % LS |
|---|---|---|
| Initial Time | 97.1 | N/A |
| −20° C. | 96.9 | 100.9 |
| 4° C. | 97.1 | 100.8 |
| 25° C./60% RH | 97.1 | 100.8 |
| 40° C./75% RH | 97.2 | 100.7 |
| 40° C./75% RH, open | 97.1 | 100.0 |
| 60° C. | 97.1 | 100.7 |

Moreover, Compound I phosphate Form I has also been found to be chemically stable up to about 40° C./75% RH for about 3 months.

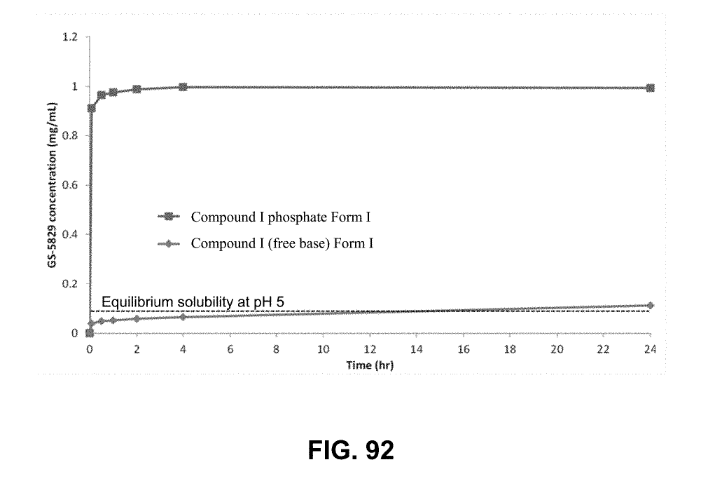

The dissolution of Compound I Form I and Compound I Form I was also evaluated in a 50 mM sodium acetate solution at a pH of 5 (FIG. 92). As particularly shown in FIG. 92, Compound I phosphate was found to dissolve rapidly and maintain solubility for about 24 hours at a about pH of 5.

### 4.1 Pharmacokinetic Data

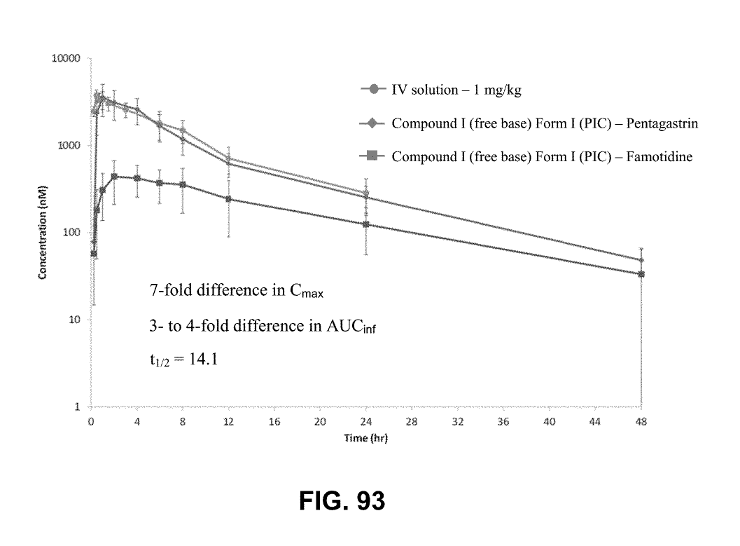

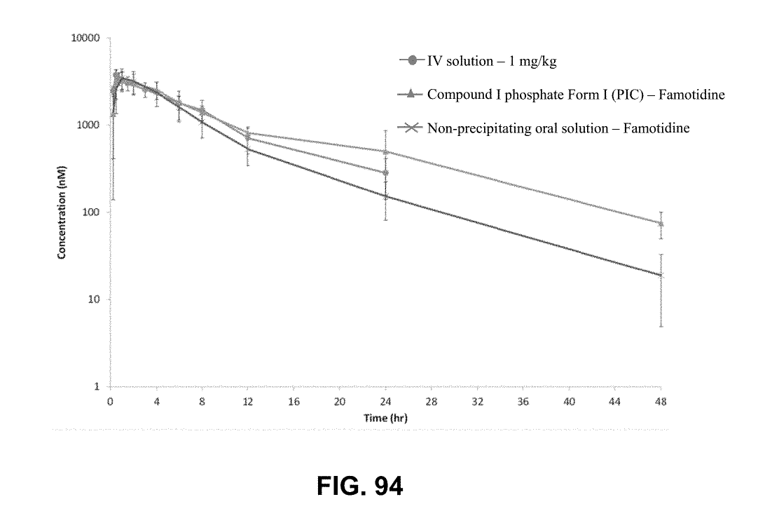

The oral bioavailability of Compound I Form I and Compound I phosphate Form I was assessed in dogs given a fixed dose of 10 mg/kg as a powder-in-capsule formulation, as shown in FIGS. 93 and 94, respectively, and as summarized in Table 4 below. Dogs pretreated with pentagastrin or famotidine were also evaluated. The bioavailability (relative to intravenous (IV) administration) of both Compound I Form I and Compound I phosphate Form I following oral administration in pentagastrin pretreated dogs was approximately 100%. In famotidine pretreated dogs, the bioavailability of Compound I Form I following oral administration was reduced to approximately 29%, indicating a potential pH effect. However, this pH effect was not observed for Compound I phosphate Form I, with approximately 100% bioavailability observed following oral administration in famotidine pretreated dogs.

TABLE 4
Pharmacokinetic Parameters in Dogs for Compound
I Form I and Compound I Phosphate Form I

| | | | | | Bioavail- |
|---|---|---|---|---|---|
| Formulation/ | | Pre- | Cₘₐₓ | AUC_{inf} | ability¹ |
| Route | Dose | treatment | (μM*hr) | (μM*hr) | (F, %) |
| Solution/IV | 1 mg/kg | None | 3940 | 31500 | — |
| Compound I | 10 mg | Pentagastrin | 3590 | 31100 | 99% |
| Form I | fixed | | | | |
| (PIC²)/Oral | 10 mg | Famotidine | 497 | 8990 | 29% |
| | fixed | | | | |
| Compound I | 10 mg | Famotidine | 3450 | 37800 | >100% |
| phosphate | fixed | | | | |
| Form I (PIC²)/ | | | | | |
| Oral | | | | | |
| Non- | 10 mg | Famotidine | 3590 | 27200 | 86% |
| precipitating | fixed | | | | |
| Solution³/ | | | | | |
| Oral | | | | | |

¹Relative to IV at 1 mg/kg dose
²Dosed as 50 wt. % Compound I solid form, 50 wt. % pre-gelatinized starch in hard gelatin capsules
³Formulation composition comprises one or more pharmaceutically acceptable vehicles such as Solutol HS-15, EtOH, polyethylene glycol, water, and HCl

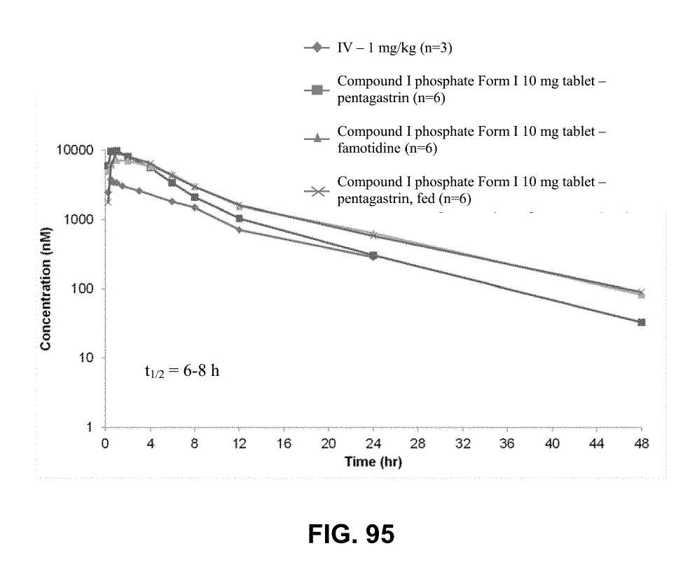

FIG. 95 provides another illustration of the oral bioavailability of Compound I phosphate Form I in dogs given a fixed dose of 10 mg as a tablet formulation.

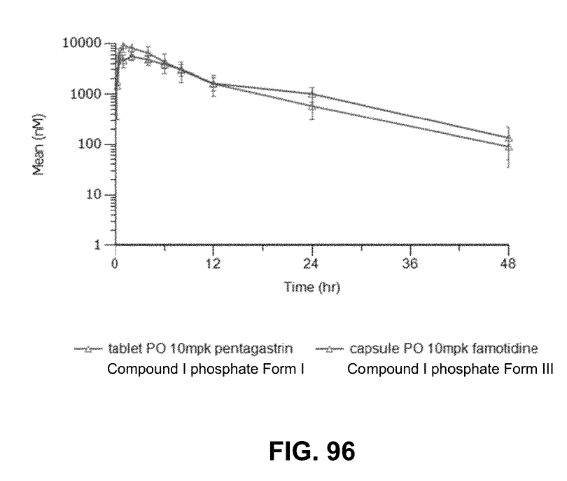

Similar oral bioavailability is contemplated for additional forms of Compound I phosphate as described herein. For instance, as shown in FIG. 96, Compound I phosphate Form III was found to exhibit similar oral bioavailability as Compound I phosphate Form I in dogs.

We claim:

1. A phosphate complex of Compound I: 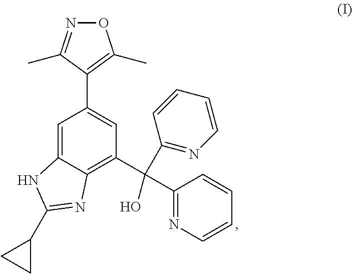 [SMILES: Cc1noc(C)c1-c1cc(C(O)(c2ccccn2)c2ccccn2)c2nc(C3CC3)[nH]c2c1]
having a crystalline form.

2. The phosphate complex of Compound I of claim 1 characterized as anhydrous.

3. A phosphate complex of Compound I: 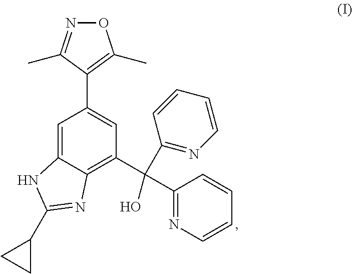 [SMILES: Cc1noc(C)c1-c1cc(C(O)(c2ccccn2)c2ccccn2)c2nc(C3CC3)[nH]c2c1]
in a crystalline form characterized by an X-ray powder diffractogram comprising peaks at 5.0, 15.8, and 21.7° 2θ±0.2° 2θ, as determined on a diffractometer using Cu-Kα radiation (Compound I phosphate Form I).

4. Compound I phosphate Form I of claim 3 further characterized by one or more of the following:
(i) one or more peaks at 12.1, 13.0, 14.9, 19.8, 23.3 and 27.0° 2θ±0.2° 2θ; and
(ii) a differential scanning calorimetry curve comprising an endotherm with onset at about 223° C.

5. Compound I phosphate Form I of claim 3 or 4 further characterized as anhydrous.

6. A phosphate complex of Compound I: 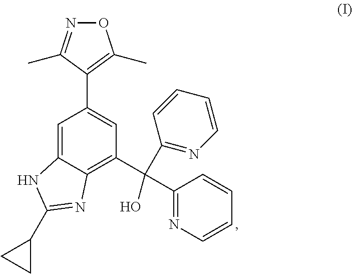 [SMILES: Cc1noc(C)c1-c1cc(C(O)(c2ccccn2)c2ccccn2)c2nc(C3CC3)[nH]c2c1]
in a crystalline form characterized by an X-ray powder diffractogram comprising peaks at 13.4, 15.0, and 20.2° 2θ±0.2° 2θ, as determined on a diffractometer using Cu-Kα radiation (Compound I phosphate Form II).

7. Compound I phosphate Form II of claim 6 further characterized by one or more of the following:
(i) one or more peaks at 5.0, 9.0, 14.1, 15.3, 19.6 and 23.0° 2θ±0.2° 2θ; and
(ii) a differential scanning calorimetry curve comprising an endotherm with onset at about 226° C.

8. A phosphate complex of Compound I: 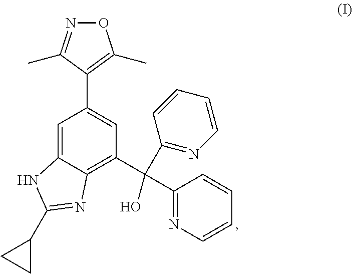 [SMILES: Cc1noc(C)c1-c1cc(C(O)(c2ccccn2)c2ccccn2)c2nc(C3CC3)[nH]c2c1]
having a crystalline form characterized by an X-ray powder diffractogram comprising peaks at 14.8, 19.7, and 24.5° 2θ±0.2° 2θ, as determined on a diffractometer using Cu-Kα radiation (Compound I phosphate Form III).

9. Compound I phosphate Form III of claim 8 further characterized by one or more of the following:
(i) one or more peaks at 5.0, 5.8, 12.7, 15.7, 16.1, 17.1, 21.9, and 22.9° 2θ±0.2° 2θ; and
(ii) a differential scanning calorimetry curve comprising endotherms with onsets at about 106° C. and about 212° C.

10. A phosphate complex of Compound I: 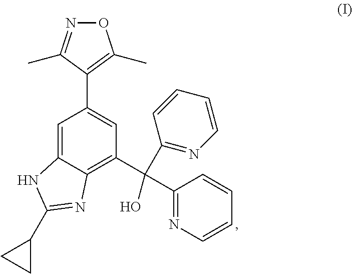 [SMILES: Cc1noc(C)c1-c1cc(C(O)(c2ccccn2)c2ccccn2)c2nc(C3CC3)[nH]c2c1]
having a crystalline form characterized by an X-ray powder diffractogram comprising peaks at 9.8, 26.5, and 29.6° 2θ±0.2° 2θ, as determined on a diffractometer using Cu-Kα radiation (Compound I phosphate Form IV).

11. Compound I phosphate Form IV of claim 10 further characterized by one or more of the following:
(i) one or more peaks at 5.0, 14.7, and 19.7° 2θ±0.2° 2θ; and
(ii) a differential scanning calorimetry curve comprising an endotherm with onset at about 211° C.

12. A phosphate complex of Compound I: 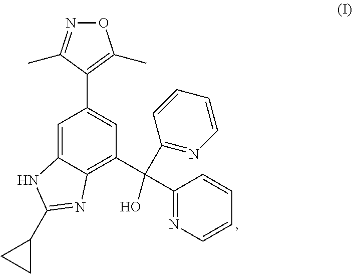 [SMILES: Cc1noc(C)c1-c1cc(C(O)(c2ccccn2)c2ccccn2)c2nc(C3CC3)[nH]c2c1]
having a crystalline form characterized by an X-ray powder diffractogram comprising peaks at 12.9, 14.0, and 22.0° 2θ±0.2° 2θ, as determined on a diffractometer using Cu-Kα radiation (Compound I phosphate Form V).

13. Compound I phosphate Form V of claim 12 further characterized by one or more of the following:
(i) one or more peaks at 5.0, 14.6, 15.0 and 21.6° 2θ±0.2° 2θ; and
(ii) a differential scanning calorimetry curve comprising endotherms with onsets at about 100° C. and about 222° C.

14. A pharmaceutical composition comprising one or more pharmaceutically acceptable carriers, and one or more compounds selected from the group consisting of:
(i) the phosphate complex of Compound I of claim 1 or 2;
(ii) Compound I phosphate Form I of any one of claims 3 to 6;
(iii) Compound I phosphate Form II of claim 6 or 7;
(iv) Compound I phosphate Form III of claim 8 or 9;
(v) Compound I phosphate Form IV of claim 10 or 11; and
(vi) Compound I phosphate Form V of claim 12 or 13.

15. A pharmaceutical composition comprising Compound I phosphate Form I and one or more pharmaceutically acceptable carriers.

16. A method of treating a disease mediated, at least in part, by a bromodomain in a patient in need thereof comprising administering a therapeutically effective amount of:
(i) the phosphate complex of Compound I of claim 1 or 2;
(ii) Compound I phosphate Form I of any one of claims 3 to 5;
(iii) Compound I phosphate Form II of claim 6 or 7;
(iv) Compound I phosphate Form III of claim 8 or 9;
(v) Compound I phosphate Form IV of claim 10 or 11;
(vi) Compound I phosphate Form V of claim 12 or 13; or
(viii) the pharmaceutical composition of claim 14 or 15.

17. The method of claim 16, wherein the bromodomain is a member of the bromodomain and extraterminal (BET) family.

18. The method of claim 16, wherein the bromodomain is BRD2, BRD3, BRD4, or BRDT.

19. The method of claim 16, wherein the disease is a cancer of the colon.

20. The method of claim 16, wherein the disease is a cancer of the prostate.

21. The method of claim 16, wherein the disease is a cancer of the breast.

22. The method of claim 16, wherein the disease is a lymphoma.

23. The method of claim 16, wherein the disease is a B-cell lymphoma.

24. The method of claim 16, wherein the disease is diffuse large B-cell lymphoma.
